SEQUENCE LISTING (138 sequences)

SEQ ID NO: 1 - DNA, 534 bp, Homo sapiens
gaagataaccggctcattcacttcctcccagaagacgcgtggtagcgagtaggcacaggcgtgcacctgctcccgaattactcaccgagacacacgggctgagcagacggcccctgtgatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaac

SEQ ID NO: 2 - DNA, 427 bp, Homo sapiens
aatgagcgccacctttaggcagatcctctgctgccagcgcagtgagaaccccaccggccccacagaaagctcagaccgctcggcttcctccctcaaccacaccatcttggctggagttcacagcaatgaccactctgtggtttagaacggaaactgagatgaggaaccagccgtcctctcttggaggataaacagcctccccctacccaattgccagggcaaggtggggtgtgagagaggagaaaagtcaactcatgtacttaaacactaaccaatgacagtatttgttcctggaccccacaagacttgatatatattgaaaattagcttatgtgacaaccctcatcttgatccccatcccttctgaaagtaggaagttggagctcttgcaatggaattcaagaacagactctggagtgtccattta

SEQ ID NO: 3 - DNA, 529 bp, Homo sapiens
ctcctctccacctgctggtgagaggtgcaaagaggcagatctttgctgccagccacttcatcccctcccagatgttggaccaacacccctccctgccaccaggcactgcctgagggcagggagtgggagccaatgaacaggcatgcaagtgagagcttcctgagctttctcctgtcggtttggtctgttttgccttcacccataagcccctcgcactctggtggcaggtgcttgtcctcagggctacagcagtagggaggtcagtgcttcgagccacgattgaaggtgacctctgccccagataggtggtgccagtggcttattaattccgatactagtttgctttgctgaccaaatgcctggtaccagaggatggtgaggcgaaggcaggttgggggcagtgttgtggcctggggccagccaacactggggctctgtatatagctatgaagaaaacacaaagttgataaatctgagtatatatttacatgtctttttaaaagggtcgttaccagagatttacccatcg

SEQ ID NO: 4 - DNA, 487 bp, Homo sapiens
aatcctaaatgtgtggtgactgctttgtagtgaactttcatatactataaactagttgtgagataacattctggtagctcagttaataaaacaatttcagaattaaagaaattttctatgcaaggtttacttctcagatgaacagtaggactttgtagttttatttccactaagtgaaaaaagaactgtgtttttaaactgtaggagaatttaataaatcagcaagggtattttagctaatagaataaaagtgcaacagaagaatttgattagtctatgaaaggttctcttaaaattctatcgaaataatcttcatgcagagatattcagggtttggattagcagtggaataaagagatgggcattgtttcccctataattgtgctgtttttataacttttgtaaatattactttttctggctgtgtttttataacttatccatatgcatgatggaaaaattttaatttgtagccatcttttcccat

SEQ ID NO: 5 - DNA, 519 bp, Homo sapiens
tttggcgtcgtcctcaagttatattagaatcgtgtcctcccggctttggccaacttactattctaggacttgattccttcattcagtcacaatttattgagcaccgactttgcatcaacctcttgctgaagataacagtgctgacaatatacagccctgccctcagagcttatatagtagaggagaaaaagtgaacccataatatacagtcagtagcgagtatttactaagtactttctatttgcgaggccctgataaaagtactgtcctggccaggcgcggtggctcacgcctgtaattccagcactttgggaggtcgaggtgggcagatcacctaaggtcaggagttcgagatcagcctggctaacatggggaaaccccgtctctactaaaaatggaaaaattagctgggcatggtggcgggcgcctgtaatcccagctactcgggaggctgagacaggagaatgacttgaacccaggagttgcagtggccaagataagatagcgccattgtactcc

SEQ ID NO: 6 - DNA, 534 bp, Mus musculus
tgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgt

SEQ ID NO: 7 - DNA, 529 bp, Homo sapiens
  modified_base (44)..(44): a, c, g, t, unknown or other
attccatgtcattctgtttacttagcacttgcactacccttgtnggttgagtgtatgctttatttgtttctagtttgaaatcccacatctgatagctgagagtaggcaaatacaacatttacctaatgtcattcactaacatggaagagttgtgaaaattctagagtgctgtaaatccttggcatacactatgacaaacaacttcattactctcccaccaggagctgctctcctgcacttagaaataatgtcacaagtagttttctaatgtacaatgcagacaaatgtactgctctctgaatacttgaagaaatggtattatacatacatagaaacttattagttataccttttcacaatcttattacgatgttgccgttaaaagggaaaaaagacacaggcaatgaatggtgggatagtaagaggacttagagtgtatgaatgagttgattttacttttttggaatttgattaagttgacagtaggcactgattggatgattaaacataagttaatctccactgtgat

SEQ ID NO: 8 - DNA, 401 bp, Homo sapiens
cttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcact

SEQ ID NO: 9 - DNA, 552 bp, Drosophila melanogaster
agagacatgtaccttgaccatcgtccttcctctcaagctagcccagagggtgggagcctaaggaagcgtggggtagcagatggagtaatggtcacgaggtccagacccactcccaaagctcagacttgccaggctccctttctcttcttccccaggtccttcctttaggtctggttgttgcaccatctgcttggttggctggcagctgagagccctgctgtgggagagcgaagggggtcaaaggaagacttgaagcacagagggctagggaggtggggtacatttctctgagcagtcagggtgggaagaaagaatgcaagagtggactgaatgtgcctaatggagaagacccacgtgctaggggatgaggggcttcctgggtcctgttcccctaccccatttgtggtcacagccatgaagtcaccgggatgaacctatccttccagtggctcgctccctgtagctctgcctccctctccatatctccttcccctacacctccctccccacacctccctactcccctgggcatcttctggcttgactggatgg

SEQ ID NO: 10 - DNA, 462 bp, Homo sapiens
gcgcgcctgctgttgcagaggagaatcctaaggagagtaaaccccaagcaggcactgcccgcccacaggatgtgaaccgcagagaccaacagaggaatccaggcacctctaccacgccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaaagctgggggatagaggggctgcagggccactggaaggaacatggagctgtcatcactcaac

SEQ ID NO: 11 - DNA, 424 bp, Homo sapiens
  modified_base (291)..(291): a, c, g, t, unknown or other
gaagcaacgttttcatctgattggaaataccgtattgctgaaaagaagaaaggcctttttaatggcttttgaacaaagcagaaaagtttgagcttctcaccttcagtcttagctcttgaacctgttgagaaagaggataagagacaaatacggaaaagagtttcagaaagcagaatctgtgtcagcccactggaaggaaaagcgaatcaaccgattcagtgatgttagtgcatccagaaacaggcttttgggaaaagcttgacctgagctgattaaatcctgaagcacaanggaagcagccacatcaaaaagttagcatgagagcagtggcgtgctcatcctctggtagcctttactgggcatttgtggagtaagagagaaaagaaaagcaggaatgttaagatatgctactaccttcaggaaa

SEQ ID NO: 12 - DNA, 383 bp, Homo sapiens
gagcagcgtagacagctggtaaactgaagagcacaactatattcttatgaaggaatttgtacctttggggtattattttgtggcccgtgaccctcgttattgttacagctgagtgtatgtttttgttctgtggagaatgctatctggcattatggtaatatattattttaggtaatatttgtactttaacatgttgcataatatatgcttatgtagctttccaggactaacagataaatgtgtaatgaacaaagatatgttgtatgagtcgtcgtttctgtcagatttgtattgtttccaagggaaaagcttgggggaggactcagttcacaaaatgcaaaactcaacgatcagattcacggacccagagcttttccatgtgt

SEQ ID NO: 13 - DNA, 200 bp, Homo sapiens
tttttctcttctggcttcataaatgccttgctgtataaattgaaatattgatactgaactgtctttttaatgatgacctaactttattcaacccatcggaatttactttttccctgaaataagatcttttccactggtctactacctgaccataaacatgtctgcatttgaattctctaaaccctaaatctgtgtctatg

SEQ ID NO: 14 - DNA, 498 bp, Mus musculus
  modified_base (105)..(105): a, c, g, t, unknown or other
aaacagagcagccacgggctcgaaccgaatccccgccgtccttagaaacggatttttttttgttttgttttgttttctggcagagtctcgatcaccaccctactnccacccccactaaggttcttgctcaatctccctagaaaacctgaattgtttcatccctttcagtcagccccctacgtggtctgaaacaaaatgaaagcacaagccacggagtttaagaaggcagcctgaaggcggggggctgaagaggggtcggggcgctgcagagtcagccaagtagccaaggaagggccccctccgcgtcgcgacggccctcgccccccgcccggcgcgcgcgcgcacaaatacacacacacagtcactcacacactcactcacactcacgccgcgctccgacaccgcctcacctctcgctccgcccgtccggcccccttccctgccccctgcgggaccggcctgcgcgcagcactggaaccacgtaggaggaggcggcgg

SEQ ID NO: 15 - DNA, 476 bp, Homo sapiens
  modified_base (235)..(236): a, c, g, t, unknown or other
gaaggtatacagactgccaacattttagacttatctctcagtctctgccactgaacttttatatatggctgctatcaaaatataaccggtttattttcatatttggaactaatataacagtatcacaaaatcttttacagtaagatagtttgtaataccagccgacccagctgcttaactgagtccttaaatcatttaatatatgggactgtaaatagagaaatctgtacattannagatctgatttctggttatgcctatagatctttattttctttatccctatagatcattttcttctgatgttaagtgttatatttttgaaatgctcctaaacaagtaagccttagattgtattaatcctgaggattgataccatttcctcaacactttgtggagtatgattgacaccagtttttttttgactgcaggtttaacttggcttatcacttttcgtattgctcagtatgtccaag

SEQ ID NO: 16 - DNA, 334 bp, Homo sapiens
cacagttttgtgaagaagcctttaatccaagttttatgagacagtaggaacctatagctacttaatttttaggagacagtaattcagttgcagaagctttcctctgctattcccattctcttttacaaaactagttttttttaaaaaatcaatgatcaattttatcttactactttataacttttgctgacttttattctttgcattgtatgtaatgtccatcagtataaattgagactgtgaatttctaggacccacaaaagtggtattttttttttgtacaagaaagtatagaggaagaggaagctgggcattaaattacctcatccagcag

SEQ ID NO: 17 - DNA, 409 bp, Homo sapiens
tatcactggtaaggagcccacgaccagacttcatttctgggaaaatgagactttgtgttgatctcatcgtgttggccttgtaaaagtgatctatgcatgtacagtgttcatgcttaatattcaagggatggggcggggaacaaaaggaatagaaagaattcttttccttgttatttggggagcacgtattgctttataactttggttgttgggagtatggctatcatataccctcatcagtgtcattttatatctgcctaattagagaaattttaaccttagtattttgatgtgttttccccattttatcctccgcaaatatctttctcttgcccattcagtgctgcttttggtttttgatttagttgtatattctggatgtatttccacagccttttattgttcttcc

SEQ ID NO: 18 - DNA, 418 bp, Mus musculus
gatccagtatgtgcttgtcttatttaaaattggaatgtgagacatgttgctgtgacctgtttttctttctcattcacatttgtagatattgtgtgaactacagtatataatgataacaattaaaaggatattctgtggatgtcacgtattttgaaatgatagaactacattagctttgtatcatgtttggataattcatcaatgttcacagtttaaaacatcattaaacattatgtaattacaatgagaaagaatcttacttaaatttggagattttcccccacatctcttttccggatacattataattctggacccctatttatctcaaaactcttaatatatgcagaccaacaggtctttgcattccttttaaataactggttgtgacaaagcttgttgttgatcagattcactg

SEQ ID NO: 19 - DNA, 149 bp, Homo sapiens
aactgctttaagtcatgcttatgctgctggtgccagtcatttgaagaaaaacagtccttggaggaaaagcagtcgtgcttaaagttcaaagctaatgatcacggatatgacaacttccgttccagtaataaatacagctcatcttgaaa

SEQ ID NO: 20 - DNA, 330 bp, Homo sapiens
tcgaggtatcagcagctctgtcctcagaatgggtcccccacttcacacagttgtaggatggctacagcagctccaagcagcacattcagaggaagaagaaaaaatgtttcatttgtgtggttttaagcataaagaagttgtttcccagagctctttgccagctgtcatccctcaaaactcactggccacaattgcttcacatgcccctgcctgaaccagccaccagagggggttaggaccaccacagattgactagatcctaaggattctctacctggggctggagttcgggtcaggtgcccgggaagcactgtgcggtgtgggaggatg

SEQ ID NO: 21 - DNA, 402 bp, Homo sapiens
  modified_base (92)..(92): a, c, g, t, unknown or other
tgttgttgtccctattgctggtttattacactgtacagaccacaaaatgtaatattcttttgtataactactaaagaaaaatccttgtagancnnnnnnccttcaccatggctatctatacctgtacatgaaatgtgtttgtattgtgctgaagngcttaatgtcaacattacctgctgnttactctgaaaaaaggaatgaatggtagctntagaatttaggatattttatcaggttggcactttataaaatactccctgatttaaaaaattgtaagttatacacgttaatcatccacattctatcgacaatgtaccaacatcacaagctgttgcaaccacctgnctgttacttctctgagctgttaaaancctggaacttcaatttcaggggggcacaaat

SEQ ID NO: 22 - DNA, 503 bp, Homo sapiens
ttacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttatttgggggtaatattgtaagttttttagaagacaattttcataacttgataaattatagttttgtttgttagaaaagtagctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaat

SEQ ID NO: 23 - DNA, 276 bp, Homo sapiens
  modified_base (193)..(193): a, c, g, t, unknown or other
aatggaccaattttagctcaacttttagtttgttagaagcaagtgtaggaactctagcactgtagtttttaattattgcttgtatctattattattaattccaacagagtataatgtatatttattctataaaatatatattatcagagtgcatttgttacaacttaggttcttttcttaccaagtattaagnaatctagtaagagnaatactagncaaaggacctagnccctgtgnaacagnntctcgtatgttatatacataaacccactctgc

SEQ ID NO: 24 - DNA, 499 bp, Homo sapiens
gtgtttctgctttacagtgctgaattccatattttagaagctatgaaagtccttttatgaaaaagttactgattgcttctcagttattaggaaaacagttgtttcacaattattatgtagatatgatgcccaaatatcatttttagtatatcttgtcgatctttaagttgttactattgtgttattcatgtctttaaatcagataccaaatattttttaggaaagaaaaatgttattactgtcattaggttgtcttttaatactttaagttattttgacgaaaagtaatagagaaaatttacttagcattttagattctagagacatggaaatgaaaattattttatgtctagagtaggtcctgaagtttggctttacattaagtttagcactgtatcagaatgaagaaactaatattttacataaaaactaatactttcaattttttatatagtaatatccccattttgtaaatgttagacttttatcatacctgtaa

SEQ ID NO: 25 - DNA, 417 bp, Mus musculus
  modified_base (112)..(112): a, c, g, t, unknown or other
aaaataggcattgcatacacatatgcacacgtatgtgcacgtgccacacattttttgtataatgttgggtttgattataaaagtgttgtcaaatgttttatttatctgcatntagcagtggttggcttttttgaattgaaatttttgcgcattgatgcattgaaataaggaaaattatttatctctgagcactaaacttatttttgcatatttctgtaatattgcagtccccagatccagaacatgggaagttagggaaaatgtgtgattttgtgttttgaattactgtcagaattacatacacaattacaacaaactttttttaaaagacatttcattgtactgcaaaaatctgaatatttatatttcttgtttttttctttatatgttttgcattttaatatgttgagccactgg

SEQ ID NO: 26 - DNA, 321 bp, Homo sapiens
tcttttgaaaatgctggcctgctgcacctgttctcagaggggcatgagaaaagaggattctcttcctagggctgtgggaagccccttgagatgttggaagcagggcagggcaggagaacccagggagggcacagagctgtggacgagggctgggaaagccatcccgcctccccagggggtctccgaggagtgctgctgtggccaaaccaggggggccactttgtgctttctgtttaggtgatgggatgcttctatctcctcagcaccccacaccaaatcccctgttattgccagatgatgtgcctaatgatcaaattgctt

SEQ ID NO: 27 - DNA, 388 bp, Homo sapiens
ctcccaccttctaggcgaatagtccccagagctgtgttcctccaaggggtccgaggaatcactcactcctggaggctggcaaggagacagtctgaggccagggacacatgaagggatgtccccaccccagcactatcagggcctccccaggcttccagagttgaaagccaggagaaaatcggcaaagaccacccttccctaaacccaagcacccaatgatgcaaaaaacaaaaacaaaaaaaaaccaccaaatccccaaattcattccagatctatttttctaccagagagaggagcaaagtcctcctcccctgcgcccttacattctgcacttcatagttggattctgagcttaggatcatctggagaccccatggagggacttgga

SEQ ID NO: 28 - DNA, 451 bp, Homo sapiens
ggtgatgttagatctggaacccccaagtgaggctggagggagttaaggtcagtatggaagatagggttgggacagggtgctttggaatgaaagagtgaccttagagggctccttgggcctcaggaatgctcctgctgctgtgaagatgagaaggtgctcttactcagttaatgatgagtgactatatttaccaaagcccctacctgctgctgggtcccttgtagcacaggagactggggctaagggcccctcccagggaagggacaccatcaggcctctggctgaggcagtagcatagaggatccatttctacctgcatttcccagaggactagcaggaggcagccttgagaaaccggcagttcccaagccagcgcctggctgttctctcattgtcactgccctctccccaacctctcctctaacccactagagattgcctgtgtcctgcc

SEQ ID NO: 29 - DNA, 457 bp, Homo sapiens
  modified_base (131)..(131): a, c, g, t, unknown or other
ccttcagaccccagttctaggggagaagagccctggacacccctgctctacccatgagcctgcccgctgcaatgcctagacttcccaacagccttagctgccagtgctggtcactaaccaacaaggttggncaccccagctaccccttctttgcagggctaaggcccccaaacatagcccctgccccggaggaagcttggggaacccatgagttgtcagctttgactttatctcctgctctttctacatgactgggcctcccttgggctggaagaattggggattctctattggaggtgagatcacagcctccagggccccccaaatcccagggaaggacttggagagaatcatgctgttgcatttagaactttctgctttgcacaggaaagagtcacacaattaatcaacatgtatattttctctatacatagagctctatttctctacggtttta

SEQ ID NO: 30 - DNA, 237 bp, Homo sapiens
  modified_base (205)..(205): a, c, g, t, unknown or other
acacggatgtggatgatgagcccccggctccagccctggcccggtcctcggagcccgtgcaggcagatgagtcccagagcccgagggatcgtgggagaatctcggctcatcagggggcccaggtggacagccgccacccccagggacagtggcgacaggacagcatgcgaacctatgaacgggaagccctgaagaaaaagtttangcgagtncatgatgcggagtcctccgatgaag

SEQ ID NO: 31 - DNA, 436 bp, Bos taurus
  modified_base (54)..(54): a, c, g, t, unknown or other
agatgtgtccaaagagatccttacattaaggtcatttgtcagaatgatgtttgngtttgtttagagttggctgacctccaactcctggggtcaaggaatcctactgccttagcttcccaaatagctaggactataggcatgcacccggccatgtgttttatttatagctcttaaagcccagatgaagaaatcacatttttgcccatagtgaagaaacatttggccattgattagtccttattttcagtgactgtcctgttttcattagattagagagaccctgtgtgggccacagttaatataaaccattatcacttttaagtaaacctgcacatcttagatttcataatttccttattgttctgactcaaaatgaactaagagcttttcactttttgtttgtaagttctcagagagtcgggtctgcaagtgcttt

SEQ ID NO: 32 - DNA, 317 bp, Mus musculus
gggtgcaggtaacccttggtctgtaagcaccaccgatccagggatcattgtctaaataggttactattgtttgtttcatcttgcttttgcatttttattttttaatttccaaattttaagtgttccctctttggggcaaattcttataaaaatgtttattgtaaagttatatattttgtctacgatgggattatgcacttcccaattgggattttacatctggatttttagtcattctaaaaaacacctaattattaaaacatttatagagtgcctactgtatgcatgagttgagttgcttctgaggtacattttga

SEQ ID NO: 33 - DNA, 431 bp, Mus musculus
gttactagactataccttggcctttaaaaatgaatctcactgattaaagagaacaggcattattaggatagcattccaccacaactagaaacattcaaataatgtgtcttaattgtaatctgtatataggaaaatttttcctatggatatttttggtgttttaccacagtgaactgatttgtagcacttatgaagtgcagaaggtaatattcttgaaaatagaaaaaggttgggtgagcaggctttaatgcctttcccccaagaatatacatcgaatttttcttaatcttttggggttggccagcttccagatttcattaataatgagctctgcctttaataaaagtacatgatcatagctacactgtatgtttaggtggtgtgaaatgatttataatcacagcttgaactgtgtttgcttggtactgtca

SEQ ID NO: 34 - DNA, 547 bp, Rattus norvegicus
  modified_base (125)..(125): a, c, g, t, unknown or other
ccctgtgcctcagtgacatgtagatgactgactgccaatacttgtcaccattccctggaagcagctacctaggggaaacaagatgtagtgctattgccgataacaagtaagattttccacactanannnnggtgtttctcttttctaaagtgaggccagtgttatttcccgggagtgttcagtcttgaccctagtcactgattttttctagttgttaatagagtggttggcttttaaggttcagagactgtggcttggcacctgcgcccaggctttgtgggcctttgccccttagaaagtagctgtaggcaaagatttgtgattttccaattacagtctcagctctagttttagtatctctaattctttggttcccttctcttccctgaaatatattagcacctgccagccaggccctcattttgcccagccagtgtgggcagatcccaccgtggagacatctgtagtgtgtatgtccttgtaacactctgttttcagggactacaacctttttccttctgtgaccagccccggattcaggctgtac

SEQ ID NO: 35 - DNA, 476 bp, Homo sapiens
ttaacactaattatcacgtctgacaaatgtgtatgtgtggtttcagttctgtgtacattttaaaggataatggtgaacattttaatgggtttcccttgccctttccatatttaacctatttccacattctctctcactcacattttctcagtgtgcccttctcttatctgccatgtccatagccataattccaccatcatacagatcaggcagtgtttaaaatgatggtaggtagcacagtggacagtctttgatcatcatgtagaatatggctatgaatcaggaaagagattagaacatttaataatgtatgtacagctggtgcttagtttttttttaatctaaatttaattaccttattggatatttgatatttggttatttaatcacagtcatctttaacagcttacactgattggtgttttatctcctgtgatcctttgatggctttttttgcctaccatttcacagaggtt

SEQ ID NO: 36 - DNA, 445 bp, Plasmodium chabaudi
cgctgggactgacgggcatgtccacctgtactccatgctgcaggcccctcccttgacttcgctgcagctctccctcaagtatctgtttgctgtgcgctggtccccagtgcggcccttggtttttgcagctgcctctgggaaaggtgacgtgcagctgtttgatctccagaaaagctcccagaaacccacagttttgatcaagcaaacccaggatgaaagccctgtctactgtctggagttcaacagccagcagactcagctcttggctgcgggcgatgcccagggcacagtgaaggtgtggcagctgagcacagagttcacggaacaagggccccgggaagctgaggacctggactgcctggcagcagaggtggcggcctgaggggtcccgggaggcgggtgcaagccttcgctgtgccgagccttgtgtttctgacgcaagcca

SEQ ID NO: 37 - DNA, 366 bp, Homo sapiens
ccccaggcgatggacaattatgatagcttatgtagctgaatgtgatacatgccgaatgccacacgtaaacacttgactataaaaactgcccccctcctttccaaataagtgcatttattgcctctatgtgcaactgacagatgaccgcaataatgaatgagcagttagaaatacattatgcttgagatgtcttaacctattcccaaatgccttctgttttccaaaggagtggtcaagcccttgcccagagctctctattctggaagagcggtccaggtggggccgggcactggccactgaattatgccagggcgcactttccactggagttcactttcaattgcttctgtgcaataaaaccaagtg

SEQ ID NO: 38 - DNA, 524 bp, Homo sapiens
  modified_base (99)..(99): a, c, g, t, unknown or other
gggtaaataacatctgacagctaatgagatattttttccatacaagataaaaagatttaatcaaaaaatttcatatttgaaatgaagtcccaaatctangttcaagttcaatagcttagccacataatacggttgtgcgagcagagaatctacctttccacttctaagcctgtttcttcctccatnnnatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgattta

SEQ ID NO: 39 - DNA, 226 bp, Homo sapiens
  modified_base (26)..(38): a, c, g, t, unknown or other
atcatcatgatcgctcgacatcgatnnnnnnnnnnnnnttttttttttttttttttttttttnttnnaagtagaaaacaaaactttatttgatgaaatctttttaaaagttccagtatgaantaacaaaatcaacaacctacaaatctctttcagtcctttgcatttcaagcaaaatattctcttcagaaaaatgaccatttcataatatatatccccttctgtcg

SEQ ID NO: 40 - DNA, 407 bp, Homo sapiens
aaaccaccaaagagagcctggcccagacatccagtaccatcactgagagcctcatggggatcagcaggatgatggcccagcaggtccagcagagcgaggaggccatgcagtctctagtcacttcttcacgaacgatcctggatgcaaatgaagaatttaagtccatgtcgggcaccatccagctgggccggaagcttatcacaaaatacaatcgccgggagctgacggacaagcttctcatcttccttgcgctacgcctgtttcttgctacggtcctctatattgtgaaaaagcggctctttccatttttgtgagatcccaaaggtgccagttctggccctttcagctcctgtttcaggatctgtcctggttcctgagctctaggctgctaagctgagccacacacc

SEQ ID NO: 41 - DNA, 453 bp, Homo sapiens
  modified_base (104)..(104): a, c, g, t, unknown or other
gaaactctggaaatcacgtgtgtggggagatggggacgcttcccatgttgtggggagctctgtggctgtgatggctgcagttgccgtgcctctgttggaacgcnaagtgcctgcaactcacgtcaatcatagaattgtgacgcacagttggcaaaatagttctttatgctatttctcaaaantttgaggacaaacccagattgggattggaatatgcactgtaaatcaaatttttcttatctacaaagactaatgtaaaaatgattttttcttctgtgcctgattaaattaactgtggtttttaatataaatatttattggtgtgctttgggagaaaaattatcttttcttgaaagaanttatcaaagcaaatttattatcttcacaagttaatgggagaatgtggttttgattctgggtgtttgaattgtgtaaacacacagcttccttgtg

SEQ ID NO: 42 - DNA, 434 bp, Homo sapiens
  modified_base (33)..(33): a, c, g, t, unknown or other
atttcccttttacattcattatgcaaattcacnttctattcntttctcacacactactagccagcctccccaaanaaggaaaagggaaaaaagtaagaaaagaatggaacaaaagaaaaataagaaagcaaacgaaaggaacaaagaaacaggataaagaaaagagatcacagatttgagaaagaaaaacaattcaattcagcaaattcaccaaaacaatgtgaatatatcctaaagtgattaaactcagaaatgatgtgaatttttccagtttacacagtttgaccaaaaacagcatggctttatgtggtagcaaaccaactgattcttgcttctactttcataagtgattttgcccacatatcatcccactttaattgttaatcagcaaaactttcaatgaaaaatcatccattttaaccaggatcacacca

SEQ ID NO: 43 - DNA, 488 bp, Homo sapiens
tctttgatcactgcctcttgattttttcctggatcattaagaggcttgaagaatactatgtagttgaaccagaggagtagtgtatgtcacatcctcacttactccttaagccctttctcatggtcttggccctaaaacatattttcagggcttgtgacccagtgatcagtggtcacccttaaagtattacagatacgtgcctgttttacatgagaggtaactgtttatgtgtataagtcatcttaataaaataacatgaaatttattagctgaattgggtagatactgcttttctaagttgaacctaacttaagctgatgcagaaactgagtcagaaaagttgctataattttaaaatataagaagtaaaagtgaaatcttatgtagcatctttatctcattttggtttgtcagtataagtttctgatttcctttaagctctttacttttagaaacgtgaatttacaatcccttatccaaaactgctg

SEQ ID NO: 44 - DNA, 169 bp, Homo sapiens
gttagtggagttttactgttaatatcatcatgtccccctttgtgtttactactgtctgaaattactgggatgtagaagcatatttcagtctgaaaattcagccagcttattttggagaagttgtatcttgttcttgggcatgttagccttgtttttcatcccaatttga

SEQ ID NO: 45 - DNA, 373 bp, Homo sapiens
atggctcgtactaaacagacagctcggaaatccaccggcggtaaagcgccacgcaagcagctggctaccaaggctgctcgcaagagcgcgccggctaccggcggtgtgaaaaagcctcaccgttaccgtccgggtactgtggctctgcgtgagatccgccgctaccaaaagtcgaccgagttgctgattcggaagctgccgttccagcgcctggtgcgagaaatcgcccaagacttcaagaccgatcttcgcttccagagctctgcggtaatggcgctgcaggaggcttgtgaggcctacttggtagggctctttgaggacacaaacctttgcgccatccatgctaagcgagtgactattatgcccaaagaca

SEQ ID NO: 46 - DNA, 223 bp, Homo sapiens
agagcaggccaaccgagcgataagtaccgttgagtttgtgacagctgctgttggtgtcttctcattcctgggcttgtttccaaaaggacaaagagcaaaatactattaacaatttaccaaagagatattgatattgaaggaatttgggaggaggaaaagaaacctggggtgaatacttattttcagtgcatcattactgttccagattcctatgatggatggc

SEQ ID NO: 47 - DNA, 535 bp, Homo sapiens
  modified_base (298)..(302): a, c, g, t, unknown or other
cagccagtggctgtggtctacagaattgtttcatataaaatacgggtagagtggtagagtttcaaaactttcgtcatagatatctgggacctttctcaggatctgtgttcacacagccaatagatttggaatcaggcctaagagtacacatggagggtaaatattaaagtgcgtattatgtacatctagaatccatgtgacttgcagcctacctgtaatttctatccattgagcatgcatggatatacccaatagtacacacaaaataaatgtttacttaagagccattctaaaaaannnnnannnaaatggtttattgtaaatctgcctaaagattttttgcatattatatatgtgaattttggttgtaagttcataacttacccaagggtatagactcataactcttttaaaacagtgcttagtacaatatcctgccatctctgtaaaaacgctaattgataaccgagtcatttacatgttttcgaacacagaatagctcttttctcagcatcattattgctctttcagcatc

SEQ ID NO: 48 - DNA, 406 bp, Homo sapiens
gattccagaatctctacctttaaacactatgttaccacttacttctcttcaaattttattgagcattagatgtttccagtatttagaagtcaaatgcttcgtttttaataggaacttacacagtcttttatgtttttttatagccctcaatgtcactgatgtggattctcccaaactcgatactttgtttgtttttatgtccccataataagtctttaagaaaacagggcaagtgagctcaaaatcaaaagaaaacccaccaacagtgaatgcattcagggctatttccaggtctttcttttgaagaaagataagactcagtccagagagcacatctgtgacacaccgtgcctcttgcctttggtgcgtggcagtcatctttggctcatgctgtacattattctac

SEQ ID NO: 49 - DNA, 507 bp, Homo sapiens
gtttcgattctgttttgttcatctgttcgagcagaggggcagttgaagtctcgtcctggtctctgccctggcatggactggcacagaggtgttctgtagttgaataggaagagcctgtctaaaaaactactgccccacttcaaattgcagtgttctgtcacctaggcatcatctcttcctgcccctagtatttgattacaaggaaccaggggaaaaaaactttcttagacacactggcaccaaggtaagaggtggggctgcccaggcaaagtcagtgaacatgaaaactcagacaaagcagagatggaaataatgcgcctcttgaggagaaaagcaataatgaataaaaggactttcctacaataacttcactgaggactcacgttaccaattttcatacttactaaagggattgtaaaaaacaccccagcattttaggtgtcttggttccatttacagcactgaggtaatctttctgctgtttgttgtcctgcttggttgagtacc

SEQ ID NO: 50 - DNA, 301 bp, Homo sapiens
ctcattattcctttacatgcagaatagaggcatttatgcaaattgaactgcagtttttcagcatatacacaatgtcttgtgcaacagaaaaacatgttggggaaatattcctcagtggagagtcgttctcatgctgacggggagaacgaaagtgacaggggtttcctcataagttttgtatgaaatatctctacaaacctcaattagttctactctacactttcactatcatcaacactgagactatcctgtctcacctacaaatgtggaaactttacattgttcgatttttcagcagact

SEQ ID NO: 51 - DNA, 515 bp, Homo sapiens
  modified_base (183)..(183): a, c, g, t, unknown or other
gaacttaagttcacacacccttgtactgcaggacggggaatggaacctaggtcttcttatttttggttcagtgttaactcccattctctaagcagactgggcctgttattcaaactgccttcccataggtgcttccctgcttctctcctcacccagagaaggacttacaaacagcttatcttncagaggttttgtgcctgatagttatggaatgtgctggtttgagcagggaggatgtaaggggagggaatgctaaaaggctgtctacttagagtcaggtttcctgggtaagtccctggaaccccatccccttcccctttcttgagaccccaggacttgctccagtaactgccaccctgtgcctttgcttcagggccatgctggataaggagctggctgcctctgtgaacatcctactcaaggcatcttcactgtgagttttgctgttgccattggaggggnngtggggggagtgtggggagtgctagggtcaggtcctggctggtgtaaagaac

SEQ ID NO: 52 - DNA, 309 bp, Homo sapiens
gactttcaccatcctgatattaaaactgtgcaggtgtccacagtagatgcttttcagggagctgaaaaggagatcattattctgtcctgtgtaaggacaagacaagtaggattcattgattcagaaaaaagaatgaatgttgcattgactagaggaaagaggcatttgttgattgtgggaaatttagcctgtttgaggaaaaatcaactttggggacgagtgatccaacactgcgaaggaagggaagatggattgcaacatgcaaaccagtatgaaccacagctgaaccatctccttaaagattatttt

SEQ ID NO: 53 - DNA, 529 bp, Homo sapiens
gtaagactggcaaggccaagatcaccggcggctatcggctcccggccaagtacgtcatccacacagtggggcccatcgcctacggggagcccagcgccagccaggctgccgagctccgcagctgctacctgagcagtctggacctgctgctggagcaccggctccgctcggtggcgttcccctgcatctccaccggcgtgtttggctacccctgtgaggcggccgccgagatcgtgctggccacgctgcgagagtggctggagcagcacaaggacaaggtggaccggctgatcatctgcgtgttcctcgagaaggacgaggacatctaccggagccggctcccccactacttccccgtggcctgaggctcccgcagcccaccctgaccgggactgacccgccttcgggaccccgctcccagctctgagaggtcgccaaagcctgcagcctggcctgggcctggccaccccttctttccctccgcgccccgcccccgaggagcctaataaagatctcgttgtggcaaaaa

SEQ ID NO: 54 - DNA, 456 bp, Homo sapiens
  modified_base (49)..(49): a, c, g, t, unknown or other
gggccccaagagcagactaggaacgcagggggctgctgctgccaggacnccacggagagccgggcacccgcctcacatgtctcctgtctggctccactgagttagccgtttgagcccactcctatcttttggtggttagtgcatcttcagctcttttctgcaagacactggaacattcctaggctgtcccaaaaggagttccaccatagcctttaaggtccgagcagggcaccaaggggttcacttttctcccgagccattcagcttggggtgcctgcgggaggggcggacagccnagccggcttcccggcggcggtacgagagcccaacaggagaggattagctgtgccaaggaacacgccactgctgcctgtctactgcccgccttctctccacttccatttttgcctttgtttttaacttgtgctcttgtgagttcttggtgtgtttctttgt

SEQ ID NO: 55 - DNA, 448 bp, Homo sapiens
acaggactatttgccacgacatttcaaaggattccaagagagaatattggtgtccatgctgtgatgattcctcagctcctctcatctgatctccgtcctggcccccatgactttctttgcggtagttagggtgtggtatgtgccactgaggcccacacctattggcaatttatagcactgatctgtcatcaataccacttgctgtcttggatgtgaagatgatttttcctgcagggattccctctacaaaattaaaaacactgggcatgtggaaataatattcacgctttaaattgtcttttctattcactacaccaggggtccccgacccctaggcaacagactgtggccctagtgtagtgaatagaaaagacaatttaaagcatgaatattatttcctcatgcccagtgtttttaattttggtactggtctgtggcttgttagaaa

SEQ ID NO: 56 - DNA, 546 bp, Homo sapiens
agccgtcagctgtctgctatgagctgcagctctgcccacttacactcaaggaacttgcaccacaagtggctgcatagccactcaggaaggccatctgcaaccagcagccccgaccagccttcccgcagccacctggacgatgatggcatgtcagtgtacacagacacgatccaacagcgcctgcgtcagattgagtcaggccaccagcaggaagtagaaactttgaagaaacaagtccaggagctgaagagtcgcctggagagccagtacctgaccagctccctacactttaatggagactttggggatgaggtgatgacccgttggcttcctgaccacctggccgcccactgctatgcgtgcgacagtgccttctggcttgccagcaggaagcaccactgcaggaattgtgggaacgtattctgctccagttgttgtaaccagaaggttccagttcccagccagcagctctttgaacccagtcgagtatgcaagtcttgctatagcagcctacatcccacaagctccagcattgaccttgaactg

SEQ ID NO: 57 - DNA, 508 bp, Homo sapiens
  modified_base (425)..(425): a, c, g, t, unknown or other
aaatgtatatatatcctagtcccctaaccaaatcctgacctattgggatacttataaaaatttaagtaagtgggatacacaaagaataataactattaacttttcattattagcaaaaacctaagggatttaaactaattgaaactgtatttgattggacttaattttttatgtttatttagaagataaagatttaaagaagacctttacaataaagagaagaaatatcgaagtcattaaaataaggagacttacttttatgacattctaatactaaaaaatatagaaatatttccttaattctagagaaactagttttactaattttttacaacttcaataataccatcactgacacttacctttattaattagcttctagaaaatagctgctaattaggttaatgaacattttaccttagtgnaaaaaaattaattaaatatgattacaaagttgcacagcataactactgagaggaaagtgattgatctgtttgtaattacttgt

SEQ ID NO: 58 - DNA, 407 bp, Homo sapiens
gtaagcaaggtctttgtggggcagttgtcgacctcaccaagacatctgaacatattcagttttcagtagaagaacttaaaaatttgttcacattacatgaaagttcagattgtgttactcatgatctgcttgactgtgagtgtacaggagaagaagttcatacaggtgattcgttggaaaaattcattgtctctagagattgtcagcttggtccacatcaccagaaatctaactccctgaaacctctttctatgtcccagctgaagcaatggaaacatttttctggagatcatttaaatcttacagatccttttcttgaaagaataacagaaaatgtgtcattcatttttcagaatataaccactcaagctactggcacatagtgaaagattacttctgacattcca

SEQ ID NO: 59 - DNA, 445 bp, Homo sapiens
acaataattagatctttttccaagttaattgggttttcccttctcccagtcataggtggtttttatcatcaagacagactgatattttgtcaggatattttcttttacagtgtttgatgtgcataatgccagagttatttttttattattcattttctctctttttgttcaatatgagattcaggatcatatttgtttaaaaggtaacacatagagatgtatgtatatattttgttataagacatacaaaataattttaagagggataaaggtgaaaatatcagattctggaaattttaagtatctaaactttatacttgtatgatttaccataaacataccaaaacatttttctgaaaatttactgtcggtctctgacatgaaaccgtattttgtcagtagttgaccaagcagttttatgagaactcttctatgcaatgatgca

SEQ ID NO: 60 - DNA, 421 bp, Homo sapiens
cttgctgcctgtctggaaagtgagaatcaaaatgctcagaggattggagcagctgcccttgggctctgatttacaattatcagaaggcaaaaacagctttgaaaagcccatcagtaaaaagaagagtggatgaagcatactccttagcaaagaaaactttcccaaactcagaagcaaaccctctaaatgcctattatttgaaatgtcttgaaaacctcgtgcagctccttaattcttcctgagtgccatgggatgctacaccttgaagctgacagtcatcaacaggggagctaaagttgaagccagctgtgtgtagcagctgttacctgaagacgtgctacctctctacaaagtgttgatccccttctttcccatgagagagagaactggtgatactccaacaccgtccagttgtggcagc

SEQ ID NO: 61 - DNA, 352 bp, Homo sapiens
caagagtagatgcagttaaggaagaaaatctgaagctaaaatcagaaaaccaagttcttggacaatatatagaaaatctcatgtcagcttctagtgtttttcaaacaactgacacaaaaagcaaaagaaagtaagggattgacacccttctgttttatggaattgctgctgatcattttttctttaaaacttggatagattccaaaagttacagtacctttgtggcttcattgaatatttatgaagataatgtcagatgtagacaaaaataacacaataacaggagacttccataagtttgtgtattatgttagtctatgaaaacgtgcaaatgtattgtagagactttatg

SEQ ID NO: 62 - DNA, 461 bp, Homo sapiens
agctgaagactctgaccaagcagatgaaggaggagaccgaggaatggaggcggttccaggcggatctgcagaccgcagtggtggtggccaatgacatcaagtgtgaggcccagcaggagctgcgcaccgtgaagaggaaactgctggaggaggaggagaagaatgcccggttgcagaaggagctgggggatgtgcagggccacggcagggtggtcaccagcagagccgcccctccctccctgggctctgtcagctagcagagcatttggtggaagaaagacagcccagctcttgccatgattgggagccgcagcccatctctagatgaaagggggaatgtgtagaggagaaattgcctctttataaagagcccagttgtctccttgtgacattctctgttctcagagtcattgccgtcgagtctctgctttttgtccacattttgggatcagcttactgca

SEQ ID NO: 63 - DNA, 349 bp, Homo sapiens
  modified_base (143)..(143): a, c, g, t, unknown or other
gaagagtgcttaaggttgaagtacaatggcacaatctcggctcacctcgacctctgcttcctgtattcaagtgattcttctgcctcagcctcccgagtagctgggattacaggcatgcgccatgacacttggctaattttttngtgtgtttttagtagagatggaattttgccatgttggccaggctggtctcaaactcctgacctcaagtgatccacccacctcgacctcccaaagtgctggtattataggcatgagccaccatgcctggcctcatttatttttaaatagctgcagtaatcccggctttagatnaaancacatgaactaataatatcactagtgttca

SEQ ID NO: 64 - DNA, 196 bp, Homo sapiens
agagctgagtcatcctagagcaaacctctggagtggagagcgaactacttcattcccctcccttagcctgggccagagagactccagctctgccttctccagccaaaaaatcaaaggcagatgggagaacagccttcagctttggataacgatgaaatatctggcaccactgatgaatattaaactttctataacc

SEQ ID NO: 65 - DNA, 387 bp, Homo sapiens
  modified_base (74)..(74): a, c, g, t, unknown or other
gagcacatatcttacaaaacaccaaaaaattcatagtgaagagaaatcaaatatacatactgagtgtggggaanccattagacaaaactcttctttttnacaacaataaaancctcacactggagagnttctctgaatgccttaagaatttggttaatatggagacccttcccagggaaacagaaggaggatcgtgaaaactgttgactacttagaatgatcacatggtttagtggagagagcatgattctgggttttaaaagtcatggatctcaatctcagctcctattactaactagatcttttactttggggtaagtcacttcatatctttaggccttaatttcctcatctgaaaaactggaaggcctgacttgttgagcttta

SEQ ID NO: 66 - DNA, 193 bp, Homo sapiens
ggcactgtgtaatcattccttgaagtagttggagatggtgctggtatgccactgaatgaggtctgagcaggttttcttcacatctgaggggacagtgccagccagtcaacttttggggtggggctgaagtctgctgaaaatctgcagttttacatgtttcatgggacattcttctgtgcaataaagtttgaga

SEQ ID NO: 67 - DNA, 480 bp, Homo sapiens
tactggtgacacttcatggctgcgacccagaatgaacttaatgcacacagggacgcagggtgtcactggtcctgggcctttgtccatgactaggtggtcagcaggacttctgcagctgactgtgcaatggctaaatgaaaagaaggccacagactaacctccactttcctgtcttcaaaattctagtgacactgggaatgctataggacctcctactattctcttaaggtcctaggaaagtttcaggaactagggaaaagactgggtactgaggctgtgtccccagatgtctgcttccgaagcagccgcgtcatgacgggtttctgctgaggaagtggtgttggcagggccccatatgccctctcgggttgtcaggggtgggagacaggctgtatgggggtccttcatgtgcagatggaacagcatcgcctcacagctgtgcagacgaacagatgtggtctactgccacgaacaatgcgg

SEQ ID NO: 68 - DNA, 427 bp, Homo sapiens
  modified_base (40)..(40): a, c, g, t, unknown or other
cctcatggtgcctcggagagtggggagcatattgggctgnggtaagcactagacccaagtagactggacacaaagggctcgcccagggccntggcgccacccccaccccttcccaccagctgctgctagcctctgtggttgtacatcccacttgcccccacacggagactgactctaaaacccttcatccaatggtgntaacccccggctntcccctgccccacctcacccacccagagaagcacagaccccgccaggggcaggggcccaccgcacacccttgtcccgggcctgtctgggactggccttcccggntcagcnagnnnnnnncagaagggacacaaagagggatggaagaaaagaacaaagagaaactgttcctcccacccccttccctgatgccaggggcaccagactgattctgagg

SEQ ID NO: 69 - DNA, 471 bp, Homo sapiens
gtcatttaccatggttttcccactgaaggctttaacttttctgataaaataatattttaaattttcaaaaacccattcctgaggagaactacttctagcattccttttcatgatgtgcttttgtgcagtaagtagcattttcggctacttaactttacattcctcttatttttcagtttccagtcaagattataaaaagcaaatgattgatataatttgatattcatagagttgtgcctacctttaatggaaaaatacatgtcagatacttagatgtttattgatatgagactatgtggttaaaaaacccaagtatgtccatgtgtttcttataaggtacacttgaaactagtgagtgtttgtcacatttcactttcatggtatataaaatgcagtttgcatatataacttgaatatctggtactagttttttcacgcctgcaatcttggagtctaggttgccttgtctct

SEQ ID NO: 70 - DNA, 323 bp, Homo sapiens
aaatccaagttcctcttactggctttcaaggatcctcctttaacttcctgttgccttgtctcatcagcaagatcataagtacctacaggtcaagcactgtctctcccttctctttagcttttcccttaggatctagcacattacccagcaaaatgtgagtagcaaggctgaaatgacatctcaataacttcaccaatgattgtaactcagcatcccttctccatcccagctgaaagcctgcttcaccatcctgcaagagatgttttttctttttgttagcatccattcccctttctaatgcagctcccaatgcataatgtgtg

SEQ ID NO: 71 - DNA, 463 bp, Homo sapiens
  modified_base (175)..(175): a, c, g, t, unknown or other
ggtcatcgaatctgcatgcatccctcatacatctggagacttcgtgaaggttccagagttactgactgagatttctgagcttttttccccttttctgtttggtttcagagtggagagcagcccaaaaatatgcaggtaactgaagccaggaaactgatttgtgttttggtttggnccggatncttnaaacagaagggaggtggagagatctgagattagaggacggggctttataggagnccaagtatggggcctgcacacacaagacacacacgcacacttgcaaacacgccacacgacacatatgcctgcatgtgtatgcacacacatgcacacgtgagctcccaaacacatcgctccttggggttacactaggtttgtttccatctggcttgaggctatttgcaggcgagagtgcagagtctgtaatgaacctcccagattctctgacgaaggggtcccc

SEQ ID NO: 72 - DNA, 525 bp, Homo sapiens
ttcctgtgtgcttagtcatcgggtccgacacaggctggactgatctggggagccgcgaagggcctgccttcacaaagggacgtaacgcaagtactgcgggcagtgtttgaatatggccctgaacaatgtgtccctgtcctccggtgatcagaggagcagggtggcctaccgctcttcccatggcgacctcagaccgcgggcgtcagcgttggcgatggtctccggagacggcttcctcgtttccaggcctgaagcgattcatctaggacctcggcaggcggtgcgaccaagcgttcgggccgagagccgtcgagtggatggtggcggccggagcccaagggaaccagatggccggggccggagccgccaagccagattctcaccttacccaatccctgccgttgaacccgatctcctaagaagtgtgctgcaacagcgtttgattgcattaggaggtgttatcgcagctcgaatttcagtttaaacgaacacctttcctctggccctcacttagcttgtgaacag

SEQ ID NO: 73 - DNA, 156 bp, Homo sapiens
gaaatgcatgggacaggtgactgacatgactgcatcgtggtttagatgtatagataacacggggaggtgctttacattttaagactttgttcataattcttttatttatggtttctctgaatcattcttttggaacattctaaaagagccagagga

SEQ ID NO: 74 - DNA, 501 bp, Homo sapiens
cgggtgaagagtgtgccggggcggcggctggctgatgggcgcacactggacgggcgggctgggctggccgacgttgcccacatactcaatggccttgctgagcagctgtggcaccaggaccaggtggcggctggcctgcttcccaaccccccagagagtgctcctgaatgagtcacgagtggttgcctgtgatcccacccccaaccctcaggtctcgacatagggctggaggctggggcaggaacatggatcctatctggaggactggccagcatggcctgatcagggaggatgtggccagagaaggcccacccgcgagcagcgctttccttgcagaattcatggcagggaggtggggaccaaggccctgagctcgaacatctcccgtggcctttccccctttggcagcaccgatggaggatgactgggagagggggtgcctctcaagttacttcaatcaagaacctgtattggttgaggtgacaccatctgttgtaacag

SEQ ID NO: 75 - DNA, 494 bp, Xenopus laevis
tgaatgaccttcctagagcacttgagcagattcattatattttaaaaccagatggagtgtttatcggtgcaatgtttggaggcgacacactctatgaacttcggtgttccttacagttagcggaaacggaaagggaaggaggattttctccacacatttctcctttcactgctgtcaatgacctgggacatctgcttgggagagctggctttaatactctgactgtggacactgatgaaattcaagttaactatcctggaatgtttgaattgatggaagatttacaaggtatgggtgagagtaactgtgcttggaatagaaaagccctgctgcatcgagacacaatgctggcagctgcggcagtgtacagagaaatgtacagaaatgaagatggttcagtacctgctacataccagatctattacatgataggatggaaatatcatgagtcacaggcaagaccagctgaaagaggttccgcaactgtgtcattt

SEQ ID NO: 76 - DNA, 369 bp, Homo sapiens
acatatgtacagtatcagtagggaaaatgtaaaaagatgttgttttcttttgtcatttaattaggccatctgtcctgttttaaagaaatagttaataattcaacactttatataacaaatattaactaatacccatatttataaaacatttttcagatttaaaagattgttaatacttataaacttagtgttattcttagaaaaccccatcaaatttaaatgtgatttacacagtgactaggaacatttgtatttattgtttcttctctgcacttttcatcatctgataaatacaagagctcaagtaactgtcttttcttcaagatggcttctatacttgaaatcagttaatacaatagtttttccagt

SEQ ID NO: 77 - DNA, 549 bp, Homo sapiens
gaggaagtcactttactactctaagatatccctaaggaatttttttttttaatttagtgtgactaaggctttatttatgtttgtgaaactgttaaggtcctttctaaattcctccattgtgagataaggacagtgtcaaagtgataaagcttaacacttgacctaaacttctattttcttaaggaagaagagtattaaatatatactgactcctagaaatctatttattaaaaaaagacatgaaaacttgctgtacataggctagctatttctaaatattttaaattagcttttctaaaaaaaaaatccagcctcataaagtagattagaaaactagattgctagtttattttgttatcagatatgtgaatctcttctccctttgaagaaactatacatttattgttacggtatgaagtcttctgtatagtttgtttttaaactaatatttgtttcagtattttgtctgaaaagaaaacaccactaattgtgtacatatgtattatataaacttaaccttttaatactgtttatttttagcccattgtt

SEQ ID NO: 78 - DNA, 515 bp, Homo sapiens
gttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtg

SEQ ID NO: 79 - DNA, 518 bp, Homo sapiens
tgatttttgtgaccctgtccatcattgttaccgtgtttgtcattaacgttcaccacagatcttcttccacgtaccaccccatggccccctgggttaagaggctctttctgcagaaacttccaaaattactttgcatgaaagatcatgtggatcgctactcatccccagagaaagaggagagtcaaccagtagtgaaaggcaaagtcctcgaaaaaaagaaacagaaacagcttagtgatggagaaaaagttctagttgcttttttggaaaaagctgctgattccattagatacatttcgagacatgtgaagaaagaacattttatcagccaggtagtacaagactggaaatttgtagctcaagttcttgaccgaatcttcctgtggctctttctgatagtgtcagtaacaggctcggttctgatttttacccctgctttgaagatgtggctacatagttaccattaggaatttaaaagacataagactaaattacaccttagacctgacatctggcta

SEQ ID NO: 80 - DNA, 137 bp, Homo sapiens
cagacacatatacaggaaagaccatgcgaagaagacacagagaggaaacggccatctacaatccaaggagagaggcctcagaacaagccaaccctgcagataccttgatctaggcatccagaattgtatgaaaatac

SEQ ID NO: 81 - DNA, 443 bp, Homo sapiens
agacaggagggccacagcgtgtggaagtaaagactttggagctagagatgccttttccagcaatgattattgacttcaccacaccccttgcctggcctggcctgaggctcagcagtgcatgacttctcgtagataacttcacagtcatccagtcccaacacctgctcttgcctggtaggaacaggcgaagtgtcagccctcaatgttgggtacttagacccaaaccaataaatggtgagttttgaacaagaactaccatcatgcaggcttcttgcccagctgaccactggccccggggtgcctgcctggctggtcttcatcacctgaggccaccaggctcaagccactgctgttgcattacacccatccctttgcaaaatccctatggagcctgtcaccactcccctccctatatacccccaccccacaaagattttcttcag

SEQ ID NO: 82 - DNA, 545 bp, Homo sapiens
  modified_base (99)..(99): a, c, g, t, unknown or other
gtgacgttggtaggaaagcatttctttacatggaggtttattttgtgggacattacctcctctggatgttacttcctcagtttacaagtagtgtaaatnctcattgattctnttatgaattgtaanggatttnctcttagcttttgagaatttagaatctganatttgaataaaaagtaaatatattcagtataattntncaaaatgctctagtttcaagatanttaaaanattatgtggaatttacataattaattcnaaatatagtttgtatttagttccnttatcaaataatgcaaatagttggnagatacctcaatttcttttgagtgttaagnaagnagncaagnaaaggnagtgaagtttttgcaacacattgtgtctttatttggtctgcctatgtttttatcacattgcttataaaacttttaaaatccttgtttgtataaaaagtttctttagttaaataaaagtgtgtgtattaattagtgtgccttctggacaaattaagaaatattttttctatatttcaatgcggttgtatt

SEQ ID NO: 83 - DNA, 424 bp, Homo sapiens
tcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggagga

SEQ ID NO: 84 - DNA, 556 bp, Homo sapiens
  modified_base (55)..(55): a, c, g, t, unknown or other
ataccacttagtatagttcgctactattttgtggcctacatgacaggtgtcaagntttttttgaatcaatttttaaaacatgccattgtgtttcaggctcgcgggattgatgtgcaacaagtgtctttggttataaattatnatcnnnctancagtcgttgaaaactatanttcacaggtgnagaagccagcatcttggctgtattgaaaaaacttcatacgtttttctactgtgatttgtatgaaaggtaacatcaaatcaaggaatagattcagtaaagtcagtagtgttcagtaagatgatgtaattaaatttgtactagggaaggttgatgagaacaaagtgggaaaacttgtaaacattgcccagattgtggacatagggttnttttccacnaattgttggtcttaccttatgcttgagcttttagtgatgttcttgtgtccatgtgtttttcttggtgattttttnctatangttgggattttcnttggtgtcgnctggtagnnnnnnnantgaaccctggtttagttatagtggctttatccctaaata

SEQ ID NO: 85 - DNA, 367 bp, Homo sapiens
ctactcgggcgctcccagaaggagccacctctcagtgcctcacctccccctgcctcccagcctccgcagatgaggttcctgccccttcctcctcgtaaccaaaaccctcactgctcccaggacggtcttatttataaaccagatacatgttcttagtctggtcccagaccaaggagctggtcagacggccctttctaatcctacatgttgagcttatgtaaaaaatgttgtttcctcctgtttttggttcctttcttacccacaaaccattactacttgaaacttaaaaaactcgccaagtgtaaaggctaaagagaagcagtttgacggaccttgtgatttgtactgtttgctgcggagctattta

SEQ ID NO: 86 - DNA, 401 bp, Homo sapiens
gagaacacctacctcttattggaaaagttggcctctcgtggaaaactgatatttttgatggctgtataaagagttgttccatgatggacgtaactcttttggatgaacatgggaaacccttttggtgtttcagttccccggtgtgcctgagatcgcctgccacaccctctgacagctctagcttcttgggacagacatacaacgtggactacgttgatgcggaaggaagagtgcacgtggagctggtgtggatcagagagaccgaagaataccttattgtcaacctggtcctttatcttagtatcgcaaaaatcaaccattggtttgggactgaatattagcagtaggtggcaaattattgttgttatttagttgtttatttttgactggctttgttcttg

SEQ ID NO: 87 - DNA, 407 bp, Homo sapiens
gccaggctttgtggtatgtacctttagtcccagctactctggaggctgaggcaggaggatcacttaagccttggaggtcaagaatgcagtgagccattatcatgccactgtgtgaccagaaaccagatgtagccatttcaagcataaaacatgatatttttgttttccttggactgaaacatagtctgggtcctcaacgttgccggtgatgatggttgaacatcatgttttttataaaccttaatttctcatttaataggaagaaaatctcaggagagccaaaagggaggacctgaaggtcagcatccaccaaatggagatggagaggatccgctacgtcctcagcagctacttgcggtgtcgcctcatgaaggtttgacgtggagatacctcaaagtctccgacct

SEQ ID NO: 88 - DNA, 439 bp, Homo sapiens
  modified_base (26)..(26): a, c, g, t, unknown or other
aaaacactactgcaatcacgtctttngttatgctagtatcagtcagatgcacttagagtgaagaaacactgtaattacagcacacagattgcaagtattgcgtaccaagtgatacaactcgaaatgcagttctcatcttcctgttttgagaaatgattattttatcacgcatcagagccttcgtgctttgattatcttgtatgttaacaattctagaaaacattcatgaattcacnaaaatangttactatggcaggggaacattttgtacacatttaagtatataaaaatactaaaatatgtaattttataacaaagtcacgggtatctttaggttcagggaactagactaggtcattcgtgtaaatggactggtagttacagtcttaggttaaagtattctaatgaagtatgggaactaaattgctggttttctaag

SEQ ID NO: 89 - DNA, 440 bp, Homo sapiens
  modified_base (90)..(93): a, c, g, t, unknown or other
ttctcttgccctttacaaattagttttctcacttaaagctatttttgttttttgctttttcactagtgaaaatgttacttcccccatgannnnanntnnnntntnntanatctacctgtgaattttgctatatttctttgttggtttggctttacaaatatgtagctgtcttctcacatgttacctgctgtaaaaccattcatttgaatcttaatagctttcacgtttacagtaacaaatgaatttccgagaaatcaagtaagttgcccaagatccaacatatataataaacatcagaactagaacttgaattctgttctttcggttgtttccaacatggactaacacattttatcaagaatgttttcaatattcaaataaggactcgaaaaaataggcttacatagtaacttttatccatcaacttacctatcgatgct

SEQ ID NO: 90 - DNA, 485 bp, Tribolium castaneum
agggagatgacactggagcaccccacagcccacaggaaagagaccagatggtcagaatggcccttaaacacatgggcagcatccaggcaccaactggagacacagccagaagggccatcatgggctttttagaagagatcctggccgtttgttttgactcatctggatcacaaggggcactcccagggttaacaaatcagtgaagatcccaccatactttctagatgtcgaaggcggcagtaggaagacctgagcttgagcataagatctgtgggatttcatcttaggggcagaaacaatccgttcactatttatttagaatgacttagcagccatttaaattttcacagagggctcaaccacctttggagtgactccatagcactggccatggtcagggttgttggaacatctgacctgtgcatccaggagccgaggagtcaggttgtaatacaggccaagcagacgggctttgagggcattta

SEQ ID NO: 91 - DNA, 320 bp, Homo sapiens
  modified_base (164)..(164): a, c, g, t, unknown or other
aaactaaacatcatatgttctcatatgtccctaagctatgaggatgcaaaggcataagaatgatacaatggactttggggactttcagggaaagggtgagaagggcgtaagggataaaagactacaaattgggttcagtatatactgctcgggtgatgggtgcnccaaaatcttaaaaatcgccaaagaacttatgtaactaaataccncctgttccccaaaaaactatggaaattaaaaattaaaaaataagtataatttctgctttagcgatattaactattcagtacncaataagtgagtttagcaattcagtgatt

SEQ ID NO: 92 - DNA, 478 bp, Homo sapiens
catactgcatgctcaggacccatagatgaactattagacatgaaatctgaggaaggtgcttgcctgggaccagtggcagggaccccggaacctgaaggtgctgacaaagatgacctgctgctgttgagtgagatcttcaatgcttcctccttggaagagggcgagttcagcaaagagtgggccgctgtgtttggagacggccaagtgaaggagccagtgcccactatggccctgggagagccagaccccaaggcccagacaggctcaggtttccttccttcgcagcttttagaccaaaatatgaaagacttacaggcctcgctacaagaacctgctaaggctgcctcagacctgactgcctggttcagcctcttcgctgacctcgacccactctcaaatcctgatgctgttgggaaaaccgataaagaacacgaattgctcaatgcatgaatctgtacccttcggagggcactcacat

SEQ ID NO: 93 - DNA, 125 bp, Homo sapiens
tctggatccaaagacgcttcggccaatgcagggattttactcatctctggcctccagtctgaggatgaggctgactattactgtatgatttggcgcggcaccgctgtggtatttggcggagggac

SEQ ID NO: 94 - DNA, 280 bp, Homo sapiens
gaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacata

SEQ ID NO: 95 - DNA, 550 bp, Homo sapiens
  modified_base (26)..(30): a, c, g, t, unknown or other
gctgcccgcagaagcgcacatgtgcnnnnnggctncnggtngggacccctnncanncantgnnccnnncgctttcctgggagaangtcactgtggacnaggttctcgagttnccattgntgaaaggncncccnnnnnnnnntntnnnncaggtgaannnnnnnnnnnnnnngcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttnnngctgcgctttccctcatcctccttctcaaaaaggatcacgcgaaagggtggctgaggctcttgaaacaggac

SEQ ID NO: 96 - DNA, 409 bp, Homo sapiens
  modified_base (53)..(53): a, c, g, t, unknown or other
tccttcttactctgcagcaacatggaggagagttttgtgtagtgagtgtgggngaagaaatacatttggctgttctcacaccccctctgactatgcaccagtgaacacatctgagtacataccagctctcctcatcttcttatttatacttaacttatttttgtgtgaaataaatggaggacgaaatcttagagcaacatcatcaaacagtctttggtccttgagaatcttctttgtgttttattttttgatttctgtagcttttcagttgcagatgttgaaattcgtaatgacaaatatgacaaattgtcatgggtgattccacttcatcttattttttctactctcactatacaatcttgcctcattttttaaaactttggaaccagaggatttcaactgcctagca

SEQ ID NO: 97 - DNA, 489 bp, Homo sapiens
  modified_base (153)..(153): a, c, g, t, unknown or other
gagactacagcagtgttacctgtgcaaatacaacttactacttctgttaccttgaacttggaaaaaaacagtgctctaccgaatgatgctgcttcaatgtcagggaaaacatctctaatttgtacacaagaagttgagaagttgaatgaggcntttgacattttgctagcttttttcatcttagcttgtgttttaatcanttttttgatctacaaagttgttcagtttaaacaaaaactaaaggcatcagaaaactcaagggaaaatagacttgaatactacagcttttatcagtcagcaaggtataatgtaactgcctcaatttgtaacacttccccaaattctctagaaagtcctggcttggagcagattcgacttcataaacaaantgttcctgaaaatgaggcacaggtcattctttttgaacattctgctttataactcaactaaatattgtctataagaaacttcagtgccatggacatgatt

SEQ ID NO: 98 - DNA, 380 bp, Homo sapiens
gatcattggtatgtcaatctcttgatgaaaaatcagtacctgaatatgtctttttgtttttttaagagacagggtcttgctatgttgcccaggcaggatttgaactcctgggatcctcccacctcagcctcccgagtacaatacctgaattttaaatagagttattgtaagtcttatgaaatgagattttgctgcactctgacataagataataaaagacagagcaggaattcattattatgagctgcttgatcagttttaaaccactccatttgatgaaacaagtgaggtccttccctcctgaccaggctgtggaatgctgtcttccccaacccccaccccctgcaaaagagcagaacaataaggcaattgctcatttt

SEQ ID NO: 99 - DNA, 562 bp, Homo sapiens
  modified_base (288)..(293): a, c, g, t, unknown or other
gcagggaaaacacagtccactccccaccaccactccgctccttccacagcaaatggggactgctcagaaaaccctgtcctttcttttcctctcttcaaaggcaggcatgtgattgaggatgtgatggtgacttctggcctgttttattttgtggtaacttcactttagtcagggaaataattggaatatctttaatctgatgtagtttgacctttagaaattgaaagtgaaacagctattgttgataattcacaaagtattaataaaacttctattacctgtaaaaannnnnnacttaatctgcgagaaaactatttagaatattatggaattgtgccatagcttctttatttgttcttaattctatattagatttttttttctctgcttcatgaacaagttcagattttaaaacattatgcctgaagacatgtccaactttattttttatatgttatattctggtccaatatactagagtaataatactctggtatttaggatatctgtcattgaacctcagttacaaattaaacaatagtagctaccatatctaagtgat

SEQ ID NO: 100 - DNA, 425 bp, Homo sapiens
agatgagattggttcccagccatttacttgctattttaatcaacatcaaagaccagatgatgtgcttctgcatcgcactcatgatgagattgtcctcctgcattgcttcctctggcccctggtgacatttgtggtgggcgttctcattgtggtcctgaccatctgtgccaagagcttggcgatcaaggcggaagccatgaagaagcgcaagttctcttaaaggggaaggaggcttgtagaaagcaaagtacagaagctgtactcatcggcacgcgtccacctgcggaacctgtgtttcctggcgcaggagatggacagggccacgacagggctctgagaggctcatccctcagtggcaacagaaacaggcacaactggaagacttggaacctcaaagcttgtattccatctgctgtagcaatggc

SEQ ID NO: 101 - DNA, 558 bp, Homo sapiens
ggatgtttatggtgaaatggcctgtacaagtttaactaagacaacttaacttgcattgttaatcaaaaattcttttctcaaagggttaactggttgccattttgaatagtatgttcaagggtgtagcttcctgtttctttccaaattataagtagctacctaaatatagtataattatatattaataatatggcttgctggcacagtagtttaccctgttatctgtgtttcataatgggggctgtatgaatattatttaaaactaataaaatgttgccagaattatactaaactgttggatgagattaggagatcagaggctggaccttctcttgataatgcttgttttgttaaaggtataatgaaataatttgtatatgatttgatgaagattaaagacccttattttccacagctttaaaaaaaaacctttatttatgatcaagtaataaagataatattctacttgtgggatcttacattatggaaatagtttgacgtttttgacctcaagagtatgtataatttgaagagatactttgtaactatgcttgggtg

SEQ ID NO: 102 - DNA, 256 bp, Homo sapiens
  modified_base (37)..(37): a, c, g, t, unknown or other
ctcaacatcataaggaatcagacggatgcggaaaccnagncgggntggatagnaaantctttccaggaaggctccggggcactcaactggtctccaaccntcccntgcaacntgtgacgcctgccatnttncccatntttaggcgantggcaacgcaanccctccgtttgctctgggcaaaacttcgagagttccctctgaagctggagctttttcctcagatccaagatccaattggtcaccaattcgtgatttc

SEQ ID NO: 103 - DNA, 409 bp, Homo sapiens
  modified_base (310)..(310): a, c, g, t, unknown or other
caagcccattttcctgagggtgaaggggacttttattatataggggccttatttggtgggtcagtgctggaggtttacaggctgatcatggcctgtcaccaggtgatgatgattgaccaagccaaccacatcgaggccctgtggcatgacgaaagcctcttaaacaagtacctgcttaaccacaaacccactctcccttgagtacatgtgggattaaaaagtcgatggagtatacgttggatgaatacctggtgggtttgtgcaccatgataaactgcaagagatctgtggtcttggtaaataacaatgnggaaatgtgaactgatgagggaagcttccagaaagagaccagagagggggtgattgccagtcagcccgnatcttcctcctgaaatgctaccctgattta

SEQ ID NO: 104 - DNA, 444 bp, Homo sapiens
  modified_base (36)..(36): a, c, g, t, unknown or other
ccttgtcaacatcttcgagcatcggcagctccggangccggggtaactggcagcaggtaggaaactatgtgaaagaatctcctgatgtcataatttccgggtgtcaccggaacatttgatcatcattcctttggcaattccagccttctgtggaaaggccagtagaaagcattgatttattcacctctacaggaatcagactcagcctcttttggttttcagtgaagtatgccttttcaatttggaacccagccaaggaggtttccagtggaaggaggagattcttcaattgagctggaacctgggctgagctccagtgctgcctgtaatgggaaggagatgtcaccaaccaggcaactccggaggtgccctggaagtcattgcctgacaataactgatgttcccgtcactgtttatgcaacaacgagaaagccacctgcacaa

SEQ ID NO: 105 - DNA, 168 bp, Homo sapiens
tgaacagagtaacaggactatatttctgaaaaaggatgaacttactggaaacaggattgcgtgcctgaaatatacccaaatggatataaatgtcaactcagttctgggctggagatatagatttggaatgcaccaacaatgcagatggtaatcctggcatgcgagtag

SEQ ID NO: 106 - DNA, 400 bp, Homo sapiens
  modified_base (97)..(97): a, c, g, t, unknown or other
ttctagaatctcagcttcttaaatcaagaaattgtgtcttgttcatgtctgaattccccaagtgaacacagtgagtgggtgcttgacaaatctttgntggtnangngcnaaaaaaggggattctgtgcccaataccatgaaatcaatgcacagaagattcantcaatcaagaaaggtgcacagacactggccacacacactgacatttgtttgcagatgttccagtccccctgacttccaccaatccattcattcattccacaagcatttttgctggggtggaagcagggccatacagggtgtgtacatcacagatagggtgggtttgtataatgagtataaaaaacttctagcagaagatgacaaagtatatcaagaaagggctctcttcgaaatcaca

SEQ ID NO: 107 - DNA, 508 bp, Homo sapiens
ggctttgggagtgagcagctagacaccaatgacgagagtgatgttatcagtgcactaagttacatttgccatatttctcagcagtaaacctagatgtggaatcaatgttactaccgttcattaaactgccaaccacaggaaacagcctggcaaagattcaaactgtaggccaaaaccggcaaaaagtgaatagagtcctcatgggcccaatgagcatccagaaaaggcacttcaaagaggtgggaaggcagagcatcaggagggaacagggtgcccaggcatctgtggagaacgctgccgaagaaaaaaggctcgggagtccagccccaagggagctggaacagcctcacacacagcaggggcctgagaagttagcgggaaacgccatctacaccaagccttcgttcagccaagagcataaggcagcagtctctgtgctgacacccttctccaagggcgcgccttctacctccagccctgcaaaagccctaccacaggtgagagacag

SEQ ID NO: 108 - DNA, 319 bp, Homo sapiens
ttatggctgggattttgcgcttagtagttcaatggcccccaggcagactacagaccgtgacaaaaggtgtggagtctcttatttgtacagattggattcgtcacaattcaccagatcaagaattccagaaaaagtgtttcaggcctcacctgaagatcatgaaaaatacggtggggatccacagaaccctcataaactgcatattgttaccagaataaaaagtacaagaagacgtccatattgggaaaaagatataataaagatgcttggattagaaaaagcacatacccctcaagttcacaagaatatcccttcagtg

SEQ ID NO: 109 - DNA, 131 bp, Homo sapiens
gttaatttgctgtgcttcttgcatttttgaaagttacatattctccactgctttaagaaataattcagttcactttcaccttggcatttcagtatctgttacacattagaagtagttgtcactatttcatc

SEQ ID NO: 110 - DNA, 398 bp, Homo sapiens
gccatgggcttcgaattgcccaccctgaagctcccatcacaggttacatgtttgggaaaggaatctactttgctgacatgtcttccaagagtgccaattactgctttgcctctcgcctaaagaatacaggactgctgctcttatcagaggtagctctaggtcagtgtaatgaactactagaggccaatcctaaggccgaaggattgcttcaaggtaaacatagcaccaaggggctgggcaagatggctcccagttctgcccacttcgtcaccctgaatgggagtacagtgccattaggaccagcaagtgacacaggaattctgaatccagatggttataccctcaactacaatgaatatattgtatataaccccaaccaggtccgtatgcggtacctt

SEQ ID NO: 111 - DNA, 335 bp, Homo sapiens
gtcagcagtcagcaaattaacatcatcatactcttccatttttagtttctgttggattttcatcaagtcaatgggctgagaaaccacttcataatagtctggttgatttcttcgctttggtgccctaatgaagagctcacagagaagtctgccctgttcatccttatagtctcggatggtattatagagttcatggcacacggcaataggatctacagttggaagattggaaagtctcctccttttcctgcttgggcctggtgttgacacagaatggtgcccatcatcaaagtccccgctgacactgctggaaggggaggtagctcttcttctct

SEQ ID NO: 112 - DNA, 420 bp, Homo sapiens
ggatgaccatgtagttgccagagcagaatatgattttgctgccgtatctgaagaagaaatttctttccgggctggtgatatgctgaacttagctctcaaagaacaacaacccaaagtgcgtggttggcttctggctagccttgatggccaaacaacaggacttatacctgcgaattatgtcaaaattcttggcaaaagaaaaggtaggaaaacggtggaatcaagtaaagtttccaagcagcaacaatcttttaccaacccaacactaactaaaggagccacggttgctgattctttggatgaacaggaagctgcctttgaatctgtttttgttgaaactaataaggttccagttgcacctgattccattgggaaagatggagaaaagcaagatctttgatatctttcatgtttgcctgc

SEQ ID NO: 113 - DNA, 487 bp, Homo sapiens
gaagtgggacgagcacatttctattgtcttcacttggatcaaaagcaaaacagtctctccgccccgcaccagatcaagtagtttggacatcaccctactgaaaacttgcgattcttcttagttttctgcatacttttcatcacgatgcaggaaacgatttcgagtcaagaagacttttatttatgaacctttgaaaggatcgtcttgtatggtgaattttctaggagcgatgatgtactgtaattttattttaatgtattttgatttatgattatttattagttttttttaaatgcttgttctaagacatttctgaatgtagaccattttccaaaaaggaaactttattttcaaaaacctaatccgtagtaattcctaatcttggagaataaaaaagggcggtggaggggaaaacattaagaatttattcattatttctcgagtactttcagaaagtctgacactttcattgttgtgccagctggtt

SEQ ID NO: 114 - DNA, 398 bp, Homo sapiens
  modified_base (156)..(157): a, c, g, t, unknown or other
cagaccctgttccttcgaggaatggggagggagggagggaccaaagccgtgaggatgaggacaactccaccctccttccttccccacaggccaaccaaccagctgctgacaggggacctggccattctcaggacaagagaatgcaggcaggcaaanngcattactgcccctgtcctnccccaccctgtcatgtgtgattccaggcaccagggcaggcccagaagcccagcagctgtgggaaggaacctgcctggggccacaggtgcccactccccaccctgcaggacaggggtgtctgtggacactcccacacccaactctgctaccaagcaggcgtctcagctttcctcctcctttaccctttcagatacaatcacgccagccacgttgttttgaaa

SEQ ID NO: 115 - DNA, 440 bp, Homo sapiens
ggcttaggcctcaaatggcttcttctaaaaactatggctctccactcatcagtggttccactccaaagcatgagcgtggctctcccagccatagtaagtcaccagcatataccccccagaatctggacagtgaaagtgagtcaggctcctccatagcagagaaatcttatcagaatagtcccagctcagacgatggtatccgaccacttccagaatacagcacagagaaacataagaagcacaaaaaggaaaagaagaaagtaaaagacaaagatagggaccgagaccgggacaaagaccgagacaagaaaaaatctcatagcatcaagccagagagttggtccaaatcacccatctcttcagaccagtccttgtctatgacaagtaacacaatcttatctgcagacagaccctcaaggctcagcccagactttatgatt

SEQ ID NO: 116 - DNA, 84 bp, Homo sapiens
gggagagggaggagtaatggaggaggagttggaaactggggagagatggaaggaatgtgactggagggtagagaacttggagaa

SEQ ID NO: 117 - DNA, 523 bp, Homo sapiens
gaatcggacatgtccaaaccaccgtgttacgaagaggcggtgctgatggcagagccgccgccgccctatagcgaggtgctcacggacacgcgcggcctctaccgcaagatcgtcacgcccttcctgagtcgccgcgacagcgcggagaagcaggagcagccgcctcccagctacaagccgctcttcctggaccggggctacacctcggcgctgcacctgcccagcgcccctcggcccgcgccgccctgcccagccctctgcctgcaggccgaccgtggccgccgggtcttccccagctggaccgactcagagctcagcagccgcgagcccctggagcacggagcttggcgtctgccggtctccatccccttgttcgggaggactacagccgtatagaggggcgcccggcgccccgggccccaccggcggactcctggcctgactgcggggctttttaaatgcttccctggactgcggggaggggcggggggagggagggatttcttatcccgtttgttacatt

SEQ ID NO: 118 - DNA, 520 bp, Homo sapiens
ttttctactcagcagatgctgtgtgttttgatgttgacagcacggtcatcagtgaagaaggaatcggatgctttcattggatttggaggaaatgtgatcaggcaacaagtcaaggataacgccaaatggtatatcactgattttgtagagctgctgggagaaccggaagaataacatccattgtcatacagctccaaacaacttcagatgaatttttacaagttacacagattgatactgtttgcttacaattgcctattacaacttgctataaaaagttggtacagatgatctgcactgtcaagtaaactacagttaggaatcctcaaagattggtttgtttgtttttaactgtagttccagtattatatgatcactatcgatttcctggagagttttgtaatctgaattctttatgtatattcctagctatatttcatacaaagtgttttaagagtggagagtcaattaaacacctttactcttaggaatatagattcggcagccttcagtgaatatt

SEQ ID NO: 119 - DNA, 389 bp, Homo sapiens
  modified_base (105)..(105): a, c, g, t, unknown or other
tagagctgaatattacttgattacaaatcagattgcttaagggtgtggaatagcaggctagttttaataccaacttgttaacataaaatcatatatgttttaganccattcttatttagttacaattttagaaagttaacaaagtaagcaggtacttatcgaagtgcatcttttcagtctaaatgtttgtctgtgtgtctaggtgctggtgagtccacatggacacatgnagnnnccatggggcaggagtctgctataaagtcagaaggtgagatcctagagagttacacccagccccattttaatttgcatgaaaagccaaggttcttttaagcactcaaattatttaatgnttaaaacacaagaaaggcacatctgttcatttaaat

SEQ ID NO: 120 - DNA, 343 bp, Homo sapiens
  modified_base (47)..(47): a, c, g, t, unknown or other
agagggccttggagtgacaccctgacccccatccactagtacttganggccagtggtggcagaagccacagaaacaagaagcccagtgagatggctaagctgcccagcatgtaacttaaatccctgttcattccccattcctttagctgctggagccagttctgcttctcggcnaggagcgatttgctggtgtagacatccgtgtccgtgtaaagggtggtggtcacgtgggcccngatttatnnnagtcccanaactgggcgcatggaggaggtggctctgggagggaggccttcacagcgctcctgtaccctttaattgtgtgtctttctcacagctatcc

SEQ ID NO: 121 - DNA, 457 bp, Homo sapiens
ggtgaatgtgtattcctctgggaggaataggaagaaaacaggaatgttaataatgtcgaacagaaaacttcctcccttattaatatataatcctcatgtatttatgcctaatgtaagctgacttttaaaaagctttcttttgttgcatgccctgtgcaggcatctgtattgtacatgcatgcctttcgtcctgttttcctgtataaagttagtgaacaaagaaatatttttgcctagttcatgttgccaagcaatgcatattttttaaatttgtcatatatggaaagagcatgtttgttacatgtaaaagctttactgatatacagatatactaatgtttgaagatgctgttctttgcaagtgtacagttttcaaatgttgttaccagtgaaacacccttgtggtttaaacttgctacaatgtatttattattcatttcctcccatgtaactaagaa

SEQ ID NO: 122 - DNA, 452 bp, Homo sapiens
tcagttcatatctttctgggcttgacatggctgatggtgtagctgaaaccctcctaacactaaaagccatttaatcttttctgtaataggagcagaaaatagttaatcatccacctagtaatataagattactgtgaatattatcttctatacattaaaacagttctagtttgtagaataataccatacaagttttatttttaaattctagttattttcagtgcttacttaaatgtaattctagaattcctccacaacttttaatattttgtatgccagtgattctcaagataaatcatgattgtagtagttgttactgttggcagtttgtagtagtattcaggtattttggggatgggggaaaacaccaaaaatcagtgtcttttatctggtgatcactgtggtatctacagtattctagtctcctgcacaaaaactgaacccactgggcc

SEQ ID NO: 123 - DNA, 551 bp, Homo sapiens
ccaaccatgatgaaactccgtctctactaaaatacaaaaattagctgggcatggttgcgtgcgcctgtagtcccagctacttgggaggctgaggcaggagaatcgcttaaacctgggagacggaggttgcagtgagccaagatcgtgtcactgcactccagcctggtgacagagtgagactctgtttcaaaaaagaaaagaaaagaaacatggttcaaattatatctaaacaaaaaagaataagaaacaaaaaacacattaaaattttaagttgtattttctatgtttctagatacatcatttttgtttgatattttcctgatgcaagtatgtggtttatcacatgtagctcttttgcatgctaaatgaaaattcaagacttgccaataaatgaatagcttattgcagacattttttaccaacattaattattttgggtttgtttaaaacctagaggcacaatcttgacttgtcaattactaccctttcacaagctaccatctcagatatatatatatatataaattcaataaagctttctgtttgtgttc

SEQ ID NO: 124 - DNA, 420 bp, Homo sapiens
cttcacaatagcaaacgtaaacgatggaattgatggaatcaaccgaaattgacggaatcaatctaaatgttcatcactgacagattgtgtaaagaaaatgtggaacatggacaccatggaatagtatgcagccataaaaagaatgagatccgatcttttgcaggaacatgcatggagccggagacagttatccttagcaaactaacgcaggaagagaaagccaaatactgcatattcttacgtataagtgggagctaaatgataagaacttatgagcacaaagtaggaaaccacagacagtggcatctccttgaggatatagggtgggagcagggagaggagcagaagagatcactattgggtactgggcttaatacctgggtgataaaataatctgtataacaaaaccccgtgacatga

SEQ ID NO: 125 - DNA, 421 bp, Homo sapiens
aaaggtgttctctgtgttatgtaaagtggaggcttccttatattttaacctactaagcaatgaggagggattcctgtcattaagcacaagggcgctggatcctcaagtgcccatcttcgtgagagaaaaagcagcacatcctgcccatttctggtgctttctgctcacaggcaccaaagctgcacatgtaaactgacttcttgccaaaggaaatgacccctgggaagttcaagctcctggaagaggctttaactcggacgcgccctcctccaggaaccagtgggcagggcagccttcatgcatgtgtaactggacctccagccataagcatggtgtgcagtatggaagagcctgctacggaactgaaagtgattggacattttataggaattgatagagatgttggtcctcaaaagctaca

SEQ ID NO: 126 - DNA, 533 bp, Homo sapiens
  modified_base (31)..(31): a, c, g, t, unknown or other
aacaacattattccatctcatttaaaggttnaaaaagaagagacaactctagccnaagtagaaatttatattctacacgtccaaactgtctcctagcagcttttggactatatatcacttgatgttaaagtatcttttatttgtaataaatattcaaatttctatttagaagctctaatgtatacctagattaaatcaaatcacagttttatgcttttaaaatatatgtatttcaaactgtatattttaatttctgagtgcatgttatatagtatttaatacttcagatgtcttggcaaattcaatataagtatttattcccacaagcgatatatgggatatctcttaaaaattatgaatatgtaccatttccttcaaagtcatcctagcctatgctgtatcaaaagtattgtatattttatggagatttagtgatatacatgtaaatgttttttaagttattttattgaagttcaatctttacataaaattaaaatctttttttaaaaaaagtgtcagtgccagaactgtaa

SEQ ID NO: 127 - DNA, 429 bp, Danio rerio
  modified_base (89)..(89): a, c, g, t, unknown or other
tgttcataaacatttgagcaccatgaaatcaaaataccctataactactttctatagtcatatctaatttatatttttttcatttccanttgtaactagatatgtagtaaagtctgaaaagactttaccatagacaataacatgcagttttatcagcaccaaagaatgttgtccaaaagaaactttttaatacctgtctttctatttataacatctgaatattttcattcttatattaagaattttgataagtagattgaatttagtatgagtactattttcttatatataccacaatggcaaacatgtattataaatcatatttttgtcttaccaattttaatatatgaggggttttagaaatttgttgtaagttatttttatattccttgtcttttgcatattttttggccaaaatcttcaatacat

SEQ ID NO: 128 - DNA, 549 bp, Homo sapiens
ttcgaggagcaagaaccacctgagaaaagtgatcctaaacaagaagagtctcagatatctgggaaggaggaagagacatcagtcaccatcttagactcttctgaggaagataaggaaaaagaagaggttgctgctgtcaaaatccaagctgccttccggggacacatagccagagaggaggcaaagaaaatgaaaacaaatagtcttcaaaatgaggaaaaagaggaaaacaagtgaggacactggttttacctccaggaaacatgaaaaataatccaaatccatcaaccttcttattaatgtcatttctccttgaggaaggaagatttgatgttgtgaaataacattcgttactgttgtgaaaatctgtcatgagcatttgtttaataagcataccattgaaacatgccacttgaagatttctctgagatcatgagtttgtttacacttgtctcaagcctatctatagagacccttggatttagaattatagaactaaagtatctgagattacagagatctcagaggttatgtgttctaactattatc

SEQ ID NO: 129 - DNA, 526 bp, Homo sapiens
gaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccatgtccaggttccctccccatatggtcccaccacatcatacgctacacacgacgggcattccgcatccggccatagtcacaccaacagtcaaacaggaatcgtcccagagtgatgtcggc

SEQ ID NO: 130 - DNA, 543 bp, Homo sapiens
tatactcactcaaggcagtgcaagatcttgaagtactttttagcagttaagtaatattgaattgtattgaatagtttacatagtttattctagtctttgaaaattactgaacatggacaatgtgcatgtcattgacatctgccttagaacttctgggacaatcctgattcgagagattctatcccattatttacatataccaaaaatactttgttaatttaatgtgttggcttcccaactcctgaacacgacacaattttattattagattttgtatggtgattttaggctatgaaaacatgatcattatatgtatatagatacatttttatttgttacaaatgtttgagcagctcactagcccacccctcctctattttgggtaagagaatttactaccttttttaactatgtagttgagagcaacatgtattttgttatttttagaatggtcagtatattgctataaaattttaaatgagactatgaaagttaaagtattctgattctggttaaattaacgaatatggttccaggccctgt

SEQ ID NO: 131 - DNA, 317 bp, Homo sapiens
gttgcagtgatgacttttgtgaaacaaaaacttatgtatcattttagtgatactcttaagattatttgttttttggcagtaaatgtgaaaattctttgttgttctactttatgaatagaacttaaggaaataactccaaaacaatgtaatttgtataagaaggttcataaaaatcctgtaaggtttaaattagtttagaagaaaaataatagtttgctgtaactttttctccctaaagaaacaaggtccaactaatccaatgctgtttcatcttgttcgagacgtcaaacaggtaagagattattttttgcttttga

SEQ ID NO: 132 - DNA, 196 bp, Homo sapiens; identical to SEQ ID NO: 64

SEQ ID NO: 133 - DNA, 287 bp, Homo sapiens
ggagactgtctcactgatgttgatttctttattcatttccgcatctgttacacgaacttcgtgtcataaattgctatcctttcatttgaaagtgtaaaaaatttcctgcatttttatcatttctgtatacttgagtttattagagattgttatgttaggcgacactgtataaaattgtatggatattttgagtgaaaatcaaaagtaaaattcacatgtatttccttttttatattttcatccaatttcttgacaacttgaataaatttcataaagagccttcctaa

SEQ ID NO: 134 - DNA, 467 bp, Homo sapiens
gcaagctctcattggctctgagcgcgaccccgcctcccaggggggtggaggtatccactgcacgtgcgccgcccgggcttcgctcagaccttcaggtgaaagctgcaaagtcgcgggtgcgtatgtacgggggctgcctcccgaggaggagctcccaagccgcagggtggacgctggagacaagaacctcagggtcacaagtttactgtttttctcccttttccatccctacattggtctgctggggaaggcggggctaggcatcactgacacacgcagactccgtggttgaggcattttattggacctttggcaattggtggtggggaggcatctgctccaactggtgcggggccctgcagatgggaccatctcaggctgggtccttgtagcccaggagcacagactggactaagcctcctgggccttgtatgaaaaaggtgttgtacctggccgtttttgccagt

SEQ ID NO: 135 - DNA, 399 bp, Homo sapiens
  modified_base (41)..(41): a, c, g, t, unknown or other
actccgtcctgggtgacaaagtgagactccctctcaaaaantaaatangtaaataaantaaatggtggtaacnatacnctatttggtaaannnnnncnctaacatctgtagtactaatcttttttccagtggctttaaactgcaaataaggaatgttgtttctgtaggtaaaatttttatttattttttcccatttaaatttacttttgttagttttttcaggcatataatatttatgttatatatggcatattctgataagaggcatacaatatgtaataatcacattagggtaaatgaggtatccatcacctttagaatttattttttgtattatgaacagttcaattgtacagttttagtttttttaaaatatacgattgttattgactacagggt

SEQ ID NO: 136 - DNA, 479 bp, Homo sapiens
tgataagactcagaacaggaagcctgcatgtgactgagcaagtcacctacataaccctgcctgtactaaggtgtacacctgtctattgtaagtttgcctaggctgttggtgtacagagaccagaggagagagacacactaggactaacaatgtcctaacaaaatggtacttagtttgttggtctttaggagaaagcattagtaatgaaagaagaaagaattttcacttggttggacattggggctgcttaagaaagttgacatttgtcgtggaatgactttggaaagacttctaaaagaatctttttcaaatccctgaaaatcaggatagcacattttgctactgactgtgacagtgttttattcttttgagagaaatgacatagttttccctttatttcccaaattcctttcatgttcttaactgctacccagaaattgagcttcagaagattgaggatagcctttgattggtattta

SEQ ID NO: 137 - DNA, 459 bp, Homo sapiens
agtagatcttaccttgctgttcataagagaatccacaatggggagaaaccctatgaatgcaatgactgtgggaaaaccttcagcagcagatcttaccttactgttcataagagaatccacaatggggagaaaccctacgaatgcagtgactgtgggaaaaccttcagcaattcctcatacctcagaccgcacttgagaattcacactggagaaaaaccgtacaaatgtaaccagtgttttcgtgagttccgcactcagtcaatcttcacaaggcacaagagagttcatacgggggagggtcattatgtatgtaatcagtgtggaaaggctttcggcacgaggtcatctctttcttcgcactatagcattcatacaggggagtacccttacgaatgccacgattgtgggagaaccttcaggaggaggtcgaatctgacacagcacataagaactcatact

SEQ ID NO: 138 - DNA, 467 bp, Homo sapiens
aagctgttgacagggctgcttttctttttggaggctctaggggagcgtctttctttgcccttccagcttctagaagctgcccaaattctgtggtttggggcctcctttcaaaaccagcaatggccaatcagtcttacatcactcaaacacttgagtgttctgtctccctcttccatgtttgaggacccttgtgattacactgtgaaaacccagataagccaggataatctccctatcttattatgaggcaagtatgttaagattttattctataatcagagaatcttatgctatgattgttatatgtgagcattatagatgctcttgaaatgttaaaatcacatcagcactggaaaataactcctaaatgtccaaaaagaacatgagatttatggtgcttgaaatgttgctaaacgtaaatttgtatctattctgaaattatataaattaacctacctggccaggca

(19) United States
(12) Patent Application Publication
(10) Pub. No.: US 2010/0256001 A1
(43) Pub. Date: Oct. 7, 2010

(54) BLOOD BIOMARKERS FOR MOOD DISORDERS

(75) Inventors: Alexander B. Niculescu, Indianapolis, IN (US); John I. Nurnberger, Indianapolis, IN (US); Daniel R. Salomon, San Diego, CA (US)

Correspondence Address: BARNES & THORNBURG LLP, P.O. BOX 2786, CHICAGO, IL, 60690-2786, US

(73) Assignee: THE SCRIPPS RESEARCH INSTITUTE, La Jolla, CA (US)

(21) Appl. No.: 12/594,378

(22) PCT Filed: Apr. 2, 2008
(86) PCT No.: PCT/US08/59120

§ 371 Date: Jun. 24, 2010

Related U.S. Application Data
(60) Provisional application No. 60/909,859, filed on Apr. 3, 2007.

(51) Int. Cl. C40B 30/00; G01N 33/68; C12Q 1/48; C12Q 1/44; C12Q 1/42; C12Q 1/02; C12Q 1/68; G01N 33/53

(52) U.S. Cl. 506/7; 436/86; 436/87; 435/15; 435/19; 435/21; 435/29; 435/6; 435/7.92

(57) ABSTRACT

A plurality of markers determine the diagnosis of a mood disorder based on their expression in a sample such as blood. Subsets of biomarkers predict the diagnosis of high or low mood disorders. The biomarkers are identified using a convergent functional genomics approach based on animal and human data. Methods and compositions for clinical diagnosis of mood disorders are provided.

### CROSS REFERENCE TO RELATED APPLICATIONS

This application claims priority to U.S. provisional application Ser. No. 60/909,859, filed Apr. 2, 2007, the disclosure of which is hereby incorporated by reference in its entirety.

Part of the work during the development of this invention was made with government support from the National Institutes of Health under grant NIMH R01 MH071912-01. The U.S. Government has certain rights in the invention.

### BACKGROUND

Research into the biological basis of mood disorders (e.g., bipolar disorders, depression) has been primarily focused in human and animal studies mostly independently. The two avenues of research have complementary strengths and weaknesses. In human genetic studies, for example, in samples of patients with mood disorders and their family members, positional cloning methods such as linkage analysis, linkage-disequilibrium mapping, and candidate-gene association analysis are narrowing the search for the chromosomal regions harboring risk genes for the illness and, in some cases, identifying plausible candidate genes and polymorphisms that will require further validation. Human postmortem brain gene expression studies have also been employed as a way of trying to identify candidate genes for mood and other neuropsychiatric disorders. In general, human studies suffer from issues of sensitivity—the signal is often difficult to detect due to the noise generated by the genetic heterogeneity of individuals and the effects of diverse environmental exposures on gene expression and phenotypic penetrance.

In animal studies, carried out in isogenic strains with controlled environmental exposure, the identification of putative neurobiological substrates of mood disorders is typically accomplished by modeling human mood disorders through pharmacological or genetic manipulations. Animal model studies suffer from issues of specificity-questions regarding direct relevance to the human disorder modeled. Each independent line of investigation (i.e., human and animal studies) is contributing to the incremental gains in knowledge of mood disorders etiology witnessed in the last decade.

However, a lack of integration between these two lines of investigation, hinders scientific understanding and slows the pace of discovery. Psychiatric phenotypes, as currently defined, are primarily the result of clinical consensus criteria rather than empirical determination. The present disclosure provides methods and compositions that empirically determine disease states for diagnosis and treatment.

Objective biomarkers of illness and treatment response would make a significant difference in the ability to diagnose and treat patients with psychiatric disorders, eliminating subjectivity and reliance of patient's self-report of symptoms. Blood gene expression profiling has emerged as a particularly interesting area of research in the search for peripheral biomarkers. Most of the studies to date have focused on human 1 lymphoblastoid cell lines (LCLs) gene expression profiling, comparison between illness groups and normal controls. They suffer from one of both of the following limitations: 1) the sample size used is often small. Given the genetic heterogeneity in human samples and the effects of illness state and environmental history, including medications and drugs, on gene expression, it may not be reliable to extract bona fide findings. 2) Use of lymphoblastoid cell lines—passaged lymphoblastoid cell lines provide a self-renewable source of material, and are purported to avoid the effects of environmental exposure of cells from fresh blood. Fresh blood, however, with phenotypic state information gathered at time of harvesting, may be more informative than immortalized lymphocytes, and may avoid some of the caveats of Epstein-Barr virus (EBV) immortalization and cell culture passaging.

The current state of the understanding of the genetic and neurobiological basis for mood disorders (such as bipolar disorder and depression) in general, and of peripheral molecular biomarkers of the illness in particular, is still inadequate. Almost all of the fundamental genetic, environmental, and biological elements needed to delineate the etiology and pathophysiology of mood disorders are yet to be completely identified, understood and validated. One of the rate-limiting steps has been the lack of concerted integration across disciplines and methodologies. The use of a multidisciplinary, integrative research framework as in the present disclosure provided herein, should lead to a reduction in the historically high rate of inferential errors committed in studies of complex diseases like bipolar disorder and depression.

Identification and validation of peripheral biomarkers for bipolar mood disorders has proven arduous, despite recent large-scale efforts. Human genomic studies are susceptible to the issue of being underpowered, due to genetic heterogeneity, the effect of variable environmental exposure on gene expression, and difficulty of accrual of large samples. Animal model gene expression studies, in a genetically homogeneous and experimentally tractable setting, can avoid artifacts and provide sensitivity of detection. Subsequent comparisons of the animal datasets with human genetic and genomic datasets can ensure cross-validatory power and specificity.

Convergent functional genomics (CFG), is an approach that translationally cross-matches animal model gene expression data with human genetic linkage data and human tissue data (blood, postmortem brain), as a Bayesian strategy of cross validating findings and identifying candidate genes, pathways and mechanisms for neuropsychiatric disorders. Predictive biomarkers for mood disorders are desired for clinical diagnosis and treatment purposes. The present disclosure provides several biomarkers that are predictive of mood disorders in clinical settings.

### SUMMARY

Methods and compositions to clinically diagnose mood disorders using a panel of biomarkers are disclosed. A panel of biomarkers may include 1 to about 100 or more biomarkers. The panel of biomarkers includes one or more biomarkers for high and low mood disorders. Blood is a suitable sample for measuring the levels or presence of one or more of the biomarkers provided herein.

In an aspect, psychiatric symptoms measured in a quantitative fashion at time of blood draw in human subjects focus on all or nothing phenomena (genes turned on and off in low symptom states vs. high symptom states). Some of the biomarkers have cross-matched animal and human data, using a convergent functional genomics approach including blood datasets from animal models.

Prioritized list of high probability blood biomarkers, provided herein, for mood state using cross-matching of animal and human data, provide a unique predictive power of the biomarkers, which have been experimentally tested.

The disclosure also provides various methods of assigning prediction scores for mood state based on the ratio of biomarkers for high mood vs. biomarkers for low mood in the blood of individual subjects, termed as BioM Mood Prediction Score. In an aspect, a panel of about 10 biomarkers, designated as BioM-10 Mood Panel, demonstrated good accuracy in predicting actual measured mood (high and low) in an enlarged cohort of subjects.

In an aspect, the present disclosure provides methods and compositions for developing clinical blood tests to quantify gene expression for diagnosis and quantitation of protein levels through immunological approaches such as enzyme-linked immunosorbent assays (ELISA).

A method of diagnosing a mood disorder in an individual includes the steps of:
(a) determining the level of a plurality of biomarkers for the mood disorder in an isolated sample from the individual, the plurality of biomarkers selected from the group of biomarkers listed in Tables 3 and 7; and
(b) diagnosing the mood state (high mood—mania, low mood—depression) in the individual based on the level of the plurality of biomarkers.

A plurality of biomarkers, in an aspect, includes a subset of about 10 markers designated as Mbp, Edg2, Fzd3, Atxn1, Ednrb for high mood and Fgfr1, Mag, Pmp22, Ugt8, Erbb3 for low mood.

A plurality of biomarkers, in an aspect, includes a subset of about 20 biomarkers designated as Mbp, Edg2, Fzd3, Atxn1, Ednrb, Pde9a, Plxnd1, Camk2d, Dio2, Lepr for high mood and Fgfr1, Mag, Pmp22, Ugt8, Erbb3, Igfbp4, Igfbp6, Pde6d, Ptprm, Nefh for low mood.

A mood disorder is a Bipolar disorder and the sample is a bodily fluid. A suitable sample is blood. The level of the biomarker may be determined in a blood sample of the individual.

In an aspect, the level of the biomarker is determined by analyzing the expression level of RNA transcripts. In an aspect, the expression level of the biomarker is determined by analyzing the level of protein or peptides or fragments thereof. Suitable detection techniques include microarray gene expression analysis, polymerase chain reaction (PCR), real-time PCR, quantitative PCR, immunohistochemistry, enzyme-linked immunosorbent assays (ELISA), and antibody arrays.

In an aspect, the determination of the level of the plurality of biomarkers is performed by an analysis of the presence or absence of the biomarkers.

A method of diagnosing mood disorder in an individual includes the steps of:
(a) performing a quantitative determination of the level of a panel of at least 10 biomarkers selected from Tables 3 and 7 in a bodily fluid sample isolated from the individual, wherein the panel comprises at least one biomarker for high mood disorder;
(b) assigning a predictive value or score to the level of the biomarkers; and
(c) diagnosing the mood disorder based on the assigned value or score.

A method of predicting the probable course and outcome (prognosis) of a mood disorder includes the steps of:
(a) obtaining a test sample from a subject, wherein the subject is suspected of having a mood disorder;
(b) analyzing the test sample for the presence or level of a plurality of biomarkers of the mood disorder, the markers selected from the group consisting of biomarkers listed in Tables 3 and 7; and
(c) determining the prognosis of the subject based on the presence or level of the biomarkers and one or more clinicopathological data to implement a particular treatment plan for the subject.

A treatment plan for a high mood disorder includes administering a pharmaceutical composition selected from a group that includes Depakote (divalproex), Lithobid (lithium), Lamictal (lamotrigene), Tegretol (carbamazepine), Topomax (topiramate).

A treatment plan for a low mood disorder includes administering a pharmaceutical composition selected from the group consisting of Prozac (fluoxetine), Zolof (sertraline), Celexa (citalopram), Cymbalta (duloxetine), Effexor (venlafaxine) or Wellbutrin (buproprion).

A clinicopathological data is selected from a group that includes patient age, previous personal and/or familial history of the mood disorder, previous personal and/or familial history of response to treatment, and any genetic or biochemical predisposition to psychiatric illness.

A suitable test sample includes fresh blood, stored blood, fixed, paraffin-embedded tissue, tissue biopsy, tissue microarray, fine needle aspirates, peritoneal fluid, ductal lavage and pleural fluid or a derivative thereof.

A method of predicting the likelihood of a successful treatment for a mood disorder in a patient includes the steps of:

(a) determining the expression level of at least 10 biomarkers, wherein the biomarkers comprise a subset of about 10 markers designated as Mbp, Edg2, Fzd3, Atxn1, Ednrb for high mood and Fgfr1, Mag, Pmp22, Ugt8, Erbb3 for low mood; and

(b) predicting the likelihood of successful treatment for the mood disorder by determining whether the sample from the patient expresses biomarkers for a high mood disorder or a low mood disorder.

A method of treating a patient suspected of suffering a mood disorder, the method includes the steps of:

(a) diagnosing whether the patient suffers from a high mood or a low mood disorder by determining the expression level of one or more of the biomarkers listed in Tables 3 and 7 in a sample obtained from the patient;

(b) selecting a treatment for the mood disorder based on the determination whether the patient suffers from a high mood or a low mood disorder; and

(c) administering to the patient a therapeutic agent capable of treating the high or the low mood disorder.

A treatment plan may be a personalized plan for the patient.

A method for clinical screening of agents capable of affecting a mood disorder, the method includes the steps of:

(a) administering a candidate agent to a population of individuals suspected of suffering from a mood disorder or induced to suffer a mood disorder;

(b) monitoring the expression profile of one or more of the biomarkers listed in Tables 3 and 7 in blood samples obtained from the individuals receiving the candidate agent compared to a control group; and

(c) determining that the candidate agent is capable of affecting the mood disorder based on the expression profile of one or more of the biomarkers in the blood samples obtained from the individuals receiving the candidate drug compared to the control.

A mood disorder microarray includes a plurality of nucleic acid molecules representing genes selected from the group of genes listed in Tables 3 and 7.

A kit for diagnosing a mood disorder includes a component selected from the group consisting of (i) oligonucleotides for amplification of one or more genes listed in Tables 3 and 7, (ii) immunohistochemical agents capable of identifying the protein products of one or more biomarkers listed in Table 7, (iii) a microarray to detect the plurality of markers listed in Tables 3 and 7, and (iv) a biomarker expression index representing the genes listed in Tables 3 and 7 for correlation.

A diagnostic microarray includes a panel of about 10 biomarkers that are predictive of a mood disorder, wherein the microarray includes nucleic acid fragments representing biomarkers designated as Mbp, Edg2, Fzd3, Atxn1, Ednrb for high mood and Fgfr1, Mag, Pmp22, Ugt8, Erbb3 for low mood.

A diagnostic antibody array includes a plurality of antibodies that recognize one or more epitopes corresponding to the protein products of the biomarkers designated as Mbp, Edg2, Fzd3, Atxn1, Ednrb for high mood and Fgfr1, Mag, Pmp22, Ugt8, Erbb3 for low mood.

A diagnostic microarray consists essentially of the top candidate markers from tables 3 and 7.

### BRIEF DESCRIPTION OF THE DRAWINGS

FIG. 1 shows Visual-Analog Mood Scale (VAS) scoring for some of the biomarkers used herein.

FIG. 2 shows prioritization (A) and conceptualization (B) of results. A: convergent functional genomics approach for candidate biomarker prioritization. Scoring of independent lines of evidence yields (maximum score=9 points). B: Conceptualization of blood candidate biomarker genes.

FIG. 3 illustrates some of the candidate biomarker genes for mood. Prioritization was based on integration of multiple lines of evidence. On the right side of the pyramid is their CFG score.

FIG. 4 is a comparison of BioM-10 Mood Prediction Score and actual mood scores in the primary cohort of bipolar subjects (n=29). BP—bipolar. Mood scores are based on subject self-report on mood VAS scale administered at time of blood draw. For biomarkers: A—called Absent by MASS analysis. P—called Present by MASS analysis. M—called Marginally Present by MASS analysis. A is scored as 0, M as 0.5 and P as 1. BioM Mood Prediction Score is based on the ratio of the sum of the scores for high mood biomarkers and sum of scores for low mood biomarkers, multiplied by 100. A cutoff score of 100 and above was used for high mood. inf—infinity-denominator is 0.

FIG. 5 is a comparison of BioM-10 Mood Prediction Score and actual mood scores in an independent cohort of psychotic disorders subjects (n=30). SZ—schizophrenia; SZA—schizoaffective disorder; SubPD—substance induced psychosis. Mood scores are based on subject self-report on mood VAS scale administered at time of blood draw. For biomarkers: A—called Absent by MASS analysis. P—called Present by MASS analysis. M—called Marginally Present by MASS analysis. A is scored as 0, M as 0.5 and P as 1. BioM Mood Prediction Score is based on the ratio of the sum of the scores for high mood biomarkers and sum of scores for low mood biomarkers, multiplied by 100. A cutoff score of 100 and above was used for high mood. inf—infinity-denominator is 0.

FIG. 6 shows Connectivity Map interrogation of drugs that have similar gene expression signatures to that of high mood. A score of 1 indicates a maximal similarity with the gene expression effects of high mood, and a score of −1 indicates a maximal opposite effects to high mood.

FIG. 7 shows Comparison of BioM-10 Mood Prediction Score and actual mood scores in a secondary independent cohort of bipolar subjects (n=19). BP—bipolar. Mood scores are based on subject self-report on mood VAS scale administered at time of blood draw. For biomarkers: A—called Absent by MASS analysis. P—called Present by MASS analysis. M—called Marginally Present by MASS analysis. A is scored as 0, M as 0.5 and P as 1. BioM Mood Prediction Score is based on the ratio of the sum of the scores for high mood biomarkers and sum of scores for low mood biomarkers, multiplied by 100. A cutoff score of 100 and above was used for high mood.

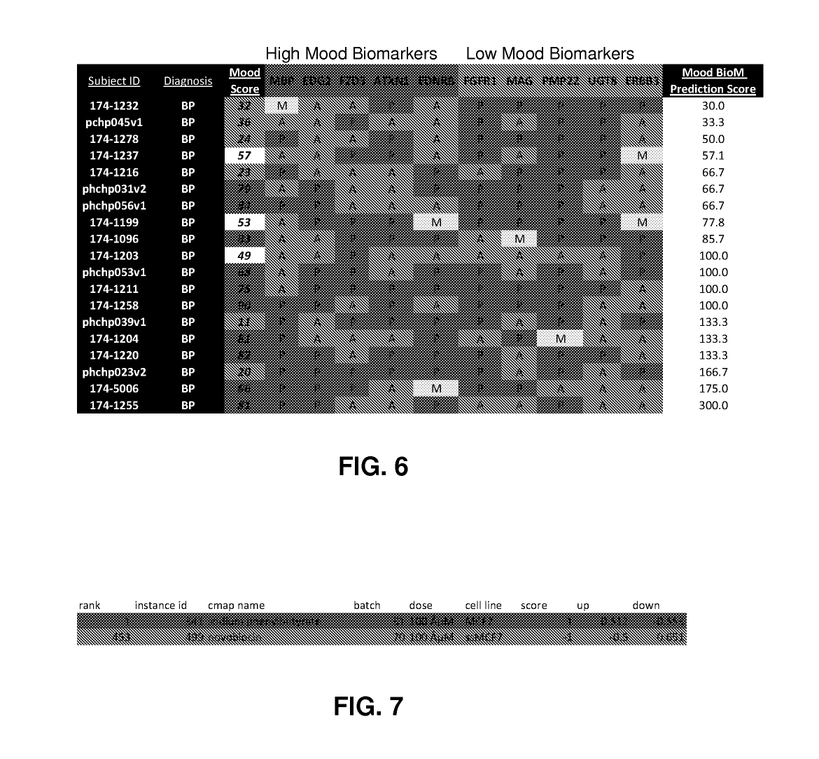

### DETAILED DESCRIPTION

Patterns of changes in the blood that reflect whether a person has low mood (depression) or high mood (mania) are disclosed. In an embodiment, these changes are analyzed at the level of gene expression, and involved genes that generally are expressed in the brain.

Unlike cancer, in psychiatric disorders, one cannot perform a biopsy the target organ (brain). Therefore, implementing a readily accessible peripheral readout in blood or any other non-brain tissue is highly useful. Blood-based screening is clinically easier to perform than a cerebro-spinal fluid (CSF) analysis or a nasal epithelium bipopsy.

Relying on the patients' self-report of symptoms and the clinician's impression of how ill the patient is alone do not necessarily provide an accurate diagnosis. Because patients' mind itself is affected in a mood disorder, their reporting of symptoms of how they feel may not be accurate or may not predict the nature of disease outcome. For example, patients aren't sure how ill they really are, and neither is the clinician—sometimes dismissing their symptoms, sometimes overestimating them. Therefore, an objective test for disease state, disease severity, and to measure response to treatment is highly desirable.

For example, for depression in general, a patient gets started on an antidepressant, and it may take weeks or months before it is known if the medication is working or if something else needs to be tried. A blood test for mood state for the biomarkers disclosed herein is able to objectively reflect whether a treatment works.

For example, for someone diagnosed as depressed but is in reality bipolar (manic-depressive), an antidepressant medication may be started only initially, and if bipolar, will be flipped by the antidepressant into a mixed state or frank mood elevation—hypomania or mania. With a panel of mood state markers, such unclear patients are monitored by repeated lab tests after the antidepressant is started, and if the markers indicate a shift beyond normal mood, to high mood, then medications can be systematically changed, a mood stabilizer added, and a potentially dangerous and certainly miserable episode for the patient averted. This approach is useful, especially in children and adolescents, who are hard to diagnose using traditional clinical criteria only, and in whom mood states rapidly change.

Sub-groups of biomarkers can be identified for different subpopulations, potential gender differences, age related differences, response to different medications. Biomarkers disclosed herein may be personalized and tailored to the individual, based on their biomarker profiles.

In an aspect, the biomarkers disclosed herein are (i) derived from fresh blood, not immortalized cell lines; (ii) capable of providing quantitative mood state information obtained at the time of the blood draw; (iii) were derived from comparisons of extremes of low mood and high mood in patients, as opposed to patients vs. normal controls (where the differences could be due to a lot of other environmental factors, medication (side) effects vs. no medications; (iv) scored based on an all or nothing (Absent/Present) call for gene expression changes, not incremental changes in expression—statistically more robust and avoids false positives; (v) based on integration of multiple independent lines of evidence that permits extraction of signal from noise (large lists of genes), and prioritization of top candidates; and (vi) used to form the basis of prediction score algorithm based.

Integration of animal model and human data was used as a way of reducing the false-positives inherent in each approach and helping identify true biomarker molecules. Gene expression differences were measured in fresh blood samples from patients with bipolar disorder (manic-depressive illness) that have low mood vs. those that have high mood at the time of the blood draw. Separately, changes in gene expression were measured in the brains and bloods of a mouse pharmacogenomic model of bipolar disorder. Human blood gene expression data was integrated with animal model gene expression data, human genetic linkage/association data, and human postmortem data for cross-validating and prioritizing findings.

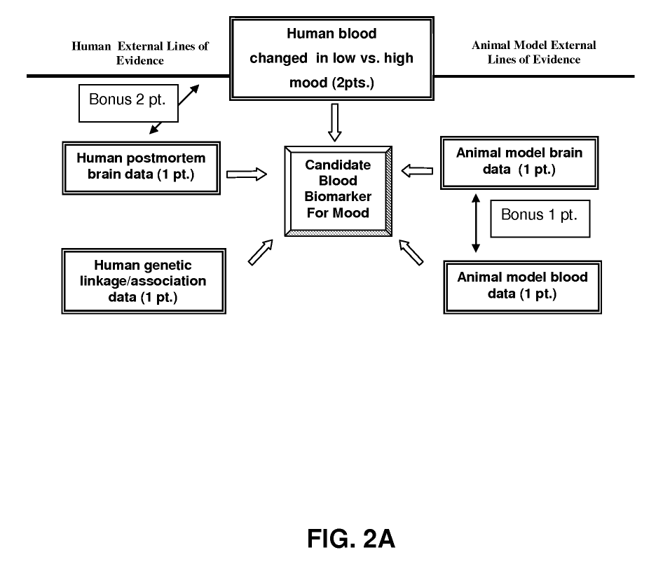

Gene expression changes in specific brain regions and blood from a pharmacogenomic animal model were used as cross-validators to identify human blood biomarkers for mood disorders. Pharmacogenomic mouse model of relevance to bipolar disorder includes treatments with an agonist of the illness/bipolar disorder-mimicking drug (methamphetamine) and an antagonist of the illness/bipolar disorder-treating drug (valproate). The pharmacogenomic approach is a tool for tagging genes that may have pathophysiological relevance. As an added advantage, some of these genes may be involved in potential medication effects present in human blood data (FIG. 2).

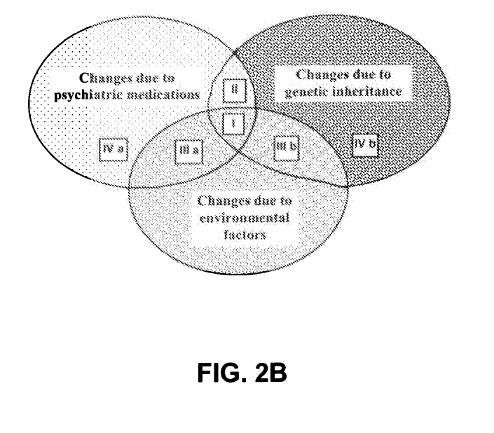

In an aspect, human whole blood gene expression studies were initially performed in a primary cohort of bipolar subjects. Whole blood was used as a way of minimizing potential artifacts related to sample handling and separation of individual cell types, and also as a way of having a streamlined approach that lends itself well to scalability, future large scale studies in the field, and easy applicability in clinical laboratory settings and doctor's offices. Genes that were differentially expressed in low mood vs. high mood subjects were compared with: 1) the results of animal model brain and blood data, as well as 2) human genetic linkage/association data, and 3) human postmortem brain data, as a way of cross-validating the findings, prioritizing them, and identifying a short list of high probability biomarker genes (FIGS. 2A and 3).

A focused approach was used to analyze discrete quantitative phenotypic item (phene)—a Visual-Analog Scale (VAS) for mood. This approach avoids the issue of corrections for multiple comparisons that would arise if one were to look in a discovery fashion at multiple phenes in a comprehensive phenotypic battery (PhenoChipping) changed in relationship with all genes on a GeneChip microarray. Larger sample cohorts would be needed for the latter approach.

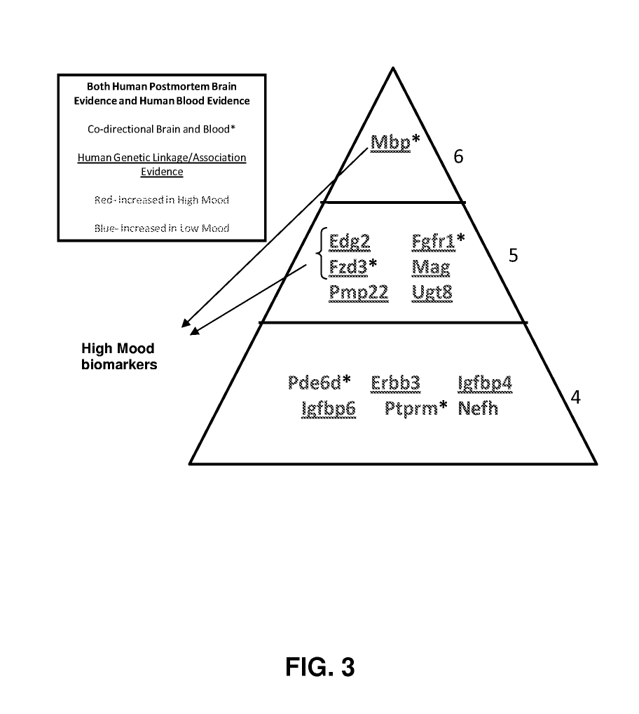

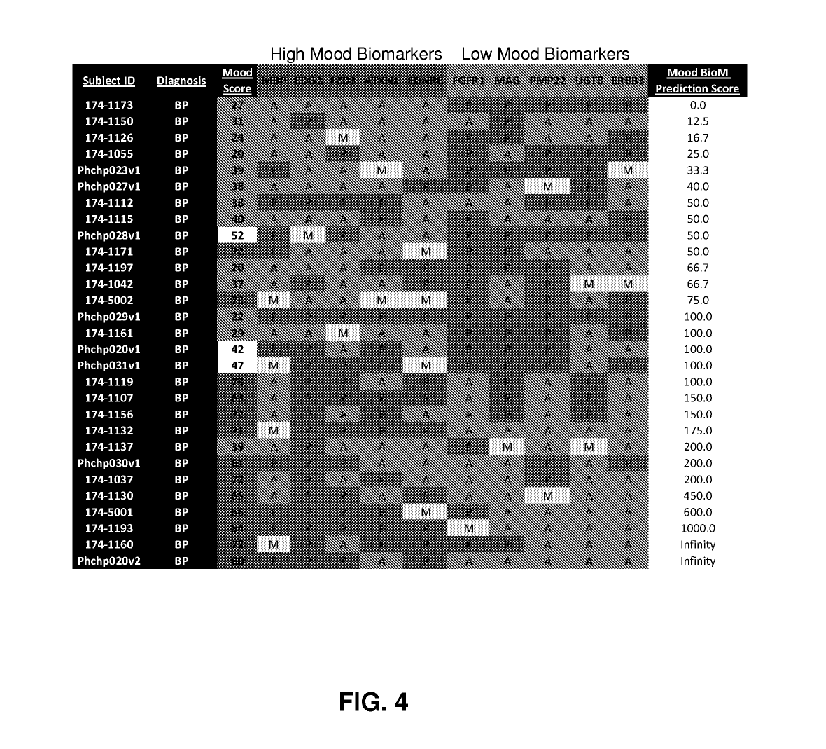

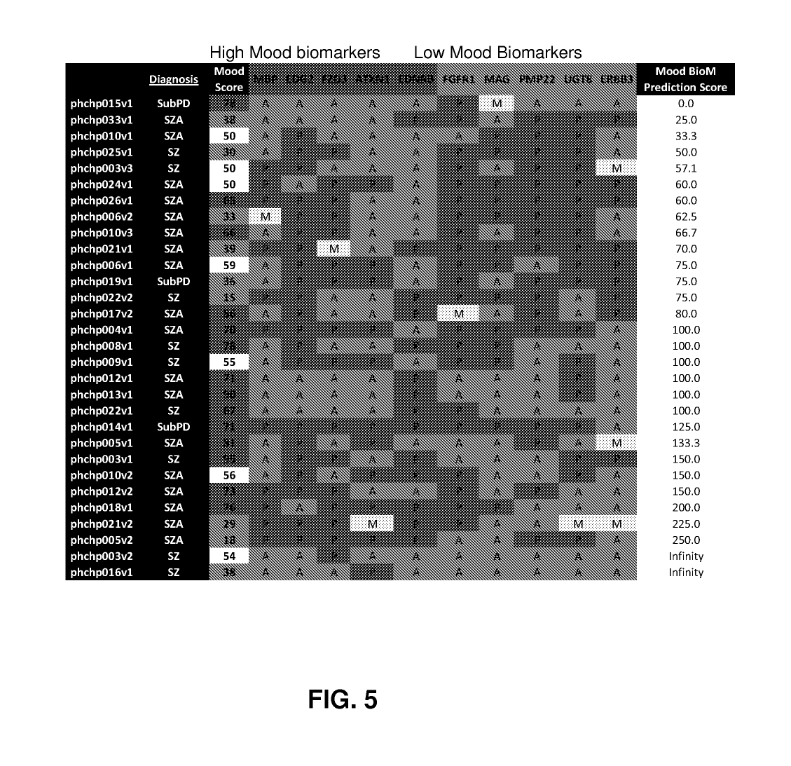

A panel of a subset of top candidate biomarker genes for mood state identified by the approach described herein was then used to generate a prediction score for mood state (low mood vs. high mood). This prediction score was compared to the actual self-reported mood scores from bipolar subjects in the primary cohort (FIG. 4). This panel of mood biomarkers and prediction score were also examined in a separate independent cohort of psychotic disorders patients for which gene expression data and mood state data (FIG. 5) were obtained, as well as in a second independent bipolar cohort (FIG. 6).

Sample size for human subjects (n=29 for the primary bipolar cohort, n=30 for the psychotic disorders cohort, n=19 for the secondary bipolar cohort) is comparable to the size of cohorts for human postmortem brain gene expression studies in the field. Live donor blood samples instead of postmortem donor brains were studied, with the advantage of better phenotypic characterization, more quantitative state information, and less technical variability. This approach also permits repeated intra-subject measures when the subject is in different mood states.

The experimental approach for detecting gene expression changes relies on a well-established methodology—oligonucleotide microarrays. To avoid the possibility that some of the gene expression changes detected from a single biological experiment are biological or technical artifacts, the analyses described herein were designed to minimize the likelihood of having false positives, even at the expense of potentially having false negatives, due to the high cost in time and resources of pursuing false leads.

For the animal model work, using isogenic mouse strain affords a suitable control baseline of saline injected animals for the drug-injected animals. Three independent de novo biological experiments were performed, at different times, with different batches of mice. This overall design is geared to factor out both biological and technical variability. It is to be noted that the concordance between reproducible microarray experiments using the latest generations of oligonucleotide microarrays and other methodologies such as quantitative PCR, with their own attendant technical limitations, is estimated to be over 90%. For the human blood samples differential gene expression analyses, which are the results of single biological experiments, it has to be noted that the approach described herein used a very restrictive and technically robust, all or nothing induction of gene expression (change from Absent Call (A) to Present Call (P)). It is possible that not all biomarker genes for mood will show this complete induction related to state, but rather some will show modulation in gene expression levels, and are thus missed by a stringent filtering approach. Moreover, given the genetic heterogeneity and variable environmental exposure, it is possible, indeed likely, that not all subjects will show changes in all the biomarker genes.

To identify candidate biomarker genes, two stringency thresholds were used: changes in 75% of subjects, and in 60% of subjects with low mood vs. high mood. Moreover, the approach, as described herein, is predicated on the existence of multiple cross-validators for each gene that is called a candidate biomarker (FIG. 2A): 1) is it changed in human blood, 2) is it changed in animal model brain, 3) is it changed in animal model blood, 4) is it changed in postmortem human brain, and 5) does it map to a human genetic linkage locus. All these lines of evidence are the result of independent experiments. The virtues of this networked Bayesian approach are that, if one or another of the nodes (lines of evidence) becomes questionable/non-functional upon further evidence in the field, the network is resilient and maintains functionality. Additional lines of evidence may move certain genes in the prioritization scoring. Using approaches described herein, a small number of genes were identified and prioritized as top biomarkers, out of the over 40,000 transcripts (about half of which are detected as Present in each subject) measured by the microarrays that were used.

A validation of the novel and non-obvious approach described herein is the fact that the biomarker panel showed sensitivity and specificity, of a comparable nature, in both independent replication cohorts (psychotic disorder cohort and secondary bipolar cohort). Thus, the approach of using a visual analog scale phene reflecting an internal subjective experience of well being or distress (as opposed to more complex and disease specific state/trait clinical instruments), and looking at extremes of state combined with robust differential expression based on A/P calls, and Convergent Functional Genomics prioritization, is able to identify state biomarkers for mood, that are, at least in part, independent of specific diagnoses or medications. Nevertheless, a comparison with existing clinical rating scales (FIG. 6), actimetry and functional neuroimaging, as well as analysis of biomarker data using such instruments may be of interest, as a way of delineating state vs. trait issues, diagnostic boundaries or lack thereof, and informing the design of clinical trials that may incorporate clinical and biomarker measures of response to treatment.

Human blood gene expression changes may be influenced by the presence or absence of both medications and drugs of abuse. While access to the subject's medical records was available and clinical information as part of the informed consent for the study, medication non-compliance, on the one hand, and substance abuse, on the other hand, are not infrequent occurrences in psychiatric patients. That medications and drugs of abuse may have effects on mood state and gene expression is in fact being partially modeled in the mouse pharmacogenomic model, with valproate and methamphetamine treatments respectively. The association of blood biomarkers with mood state is analyzed, regardless of the proximal causes, which could be diverse (see FIG. 2B). The performance the biomarkers identified herein can also be analyzed at a protein level, in larger cohorts of both genders, in different age groups, and in theragnostic settings—measuring responses to specific treatments/medications.

A subset of top candidate biomarker genes include five genes involved in myelination (Mbp, Edg2, Mag, Pmp22 and Ugt8), six genes involved in growth factor signaling (Fgfr1, Fzd3, Erbb3, Igfbp4, Igfbp6), one gene involved in light transduction (PDE6D), and one gene involved in neurofilaments (Nefh). These genes were selected as having a line of evidence (CFG) score of 4 or higher (Table 3). That means, in addition to the human blood data, these genes have at least two other independent lines of evidence implicating them in mood disorders and/or concordance of expression in human brain and blood. Using this cutoff score, about 13 genes (FIG. 3), all of which have evidence of differential expression in human postmortem brains from mood disorder patients.

It is intriguing that genes which have a well-established role in brain functioning may show changes in blood in relationship to psychiatric symptoms state (FIG. 3, Table 3 and Table 7), and moreover that the direction of change may be concordant with that found in human postmortem brain studies. It is possible that trait promoter sequence mutations or epigenetic modifications influence expression in both tissues (brain and blood), and that state dependent transcription factor changes that modulate the expression of these genes may be contributory as well.

The data provided herein demonstrate that genes involved in brain infrastructure changes (myelin, growth factors) are prominent players in mood disorders, and are reflected in the blood profile. Myelin abnormalities have emerged as a common if perhaps non-specific denominator across a variety of neuropsychiatric disorders. For example, Mbp, is a top scoring candidate biomarker (FIG. 3), associated with high mood state. The data provided herein regarding insulin growth factor signaling changes may provide an underpinning for the co-morbidity with diabetes and metabolic syndrome often encountered in mood disorder patients. These changes may be etiopathogenic, compensatory mechanisms, side-effects of medications, or results of illness—induced lifestyle changes (FIG. 2B).

The fact that many of the biomarkers identified are associated with a low mood state (depression) as opposed to high mood state (FIG. 3 and Table 3) indicates that depression may have more of an impact on blood gene expression changes, perhaps through a neuro-endocrine-immunological axis, as part of a whole-body reaction to a perceived hostile environment.

Some of the candidate biomarker genes identified herein have no previous evidence for involvement in mood disorders (Tables 3 and 7). They merit further exploration in genetic candidate gene association studies, as well as comparison with emerging results from whole—genome association studies of bipolar disorder and depression. If needed, the composition of biomarker panels for mood can be refined or changed for different sub-populations, depending upon the availability of additional evidence. Panels containing different number of biomarkers and different biomarkers can be developed using the guidelines described herein and from the biomarkers identified herein. A large number of the biomarkers that would be of use in different panels and permutations are already present in the complete list of candidate biomarker genes identified (Tables 3 and 7).

An interrogation of a connectivity map with a signature query composed of the genes in a panel of top biomarkers for low mood and high mood revealed that sodium phenylbutyrate exerts the most similar effects to high mood, and novobiocin the most similar effects to low mood (FIG. 5). Sodium phenylbutirate is a medication used to treat hyperammonemia that also has histone deacetylase (HDAC) properties, cell survival and anti-apoptotic effects. The mood stabilizer drug valproate, also a HDAC inhibitor, as well as sodium phenylbutirate and another HDAC inhibitor, trichostatin A, were shown to induce alpha-synuclein in neurons through inhibition of HDAC and that this alpha-synuclein induction was critically involved in neuroprotection against glutamate excitotoxicity. Human postmortem brain studies, as well as animal model and clinical studies have implicated glutamate abnormalities and histone deacetylase modulation as therapeutic targets in mood disorders. Novobiocin is an antibiotic drug that also has anti-tumor activity and apoptosis-inducing properties, through Hsp90 inhibition of Akt kinase an effect opposite to that of the valproate, trichostatin A and sodium phenylbutyrate (Table 6).

This connectivity map analysis with a mood panel genes provides an interesting external biological cross-validation for the internal consistency of the biomarker approach, as well as illustrates the utility of the connectivity map for non-hypothesis driven identification of novel drug treatments and interventions.

The results provided herein are consistent with a trophicity model for genes involved in mood regulation: cell survival and proliferation associated with high mood, and cell shrinkage and death associated with low mood. This perspective is both of evolutionary interest and pragmatic clinical importance. Nature may have selected primitive cellular mechanisms for analogous higher organism level-functions: survival and expansion in favorable, mood-elevating environments, withdrawal and death (apoptosis) in unfavorable, depressogenic environments. In this view, suicide is the organismal equivalent of cellular apoptosis (programmed cell death). Pragmatically, the results point to an unappreciated molecular and therapeutic overlap between two broad areas of medicine: mood disorders and cancer. This overlap is relevant for the clinical co-morbidity of mood disorders and cancer, as well as for empirical studies to evaluate the use of mood-regulating drugs in cancer, and of cancer drugs in mood disorders.

In clinical practice there is a high degree of overlap and co-morbidity between mood disorders, psychosis and substance abuse. The data in bipolar and psychotic disorder cohorts point to the issue of heterogeneity, overlap and interdependence of major psychiatric syndromes as currently defined by DSM-IV, and the need for a move towards comprehensive empirical profiling and away from categorical diagnostic classifications.

There are to date no reliable clinical laboratory blood tests for mood disorders. A translational convergent approach is disclosed herein to identify and prioritize blood biomarkers of mood state. Data demonstrate that blood biomarkers are effective in offering an unexpectedly informative window into brain functioning and disease state. Panels of such biomarkers may serve as a basis for objective clinical laboratory tests, a longstanding unmet need for psychiatry. Biomarker-based tests are extremely valuable for early intervention and prevention efforts, as well as monitoring response to various treatments. In conjunction with other relevant clinical information, biomarker tests play a desirable part of personalizing treatment to increase effectiveness and avoid adverse reactions—personalized medicine in psychiatry. Moreover, the biomarkers identified herein are useful for identifying or screening new neuropsychiatric drugs, at both a pre-clinical and clinical (Phase I, II and III) stages of the process.

Because brain is a highly specialized organ, it is not expected that the genes expressed in the brain would be present in the blood. Expression products of genes (e.g., RNA and protein) are generally tissue specific and are not expected or predicted to be expressed in an unrelated tissue, e.g., blood. Therefore, the finding that certain markers are expressed in blood and are predictable for mood disorder in patients is surprising and non-obvious. Not all markers differentially expressed in blood and are associated with predicting/diagnosing mood disorder are expressed in the brain. Similarly, not all genes that are differentially expressed in brain are expressed in blood for predicting/diagnosing mood disorder.

Human postmortem brain gene expression studies are generally susceptible to the issue of being underpowered, due to uncertainty of diagnosis and difficulty of accrual of large well-characterized cohorts, as well as due to genetic heterogeneity and the effect of variable environmental exposure on gene expression. Moreover, postmortem work artifacts (agonal interval, pH and tissue degradation) may influence gene expression changes.

For example, the data presented herein has not found reliable blood evidence for some of the top candidate genes derived from postmortem work, such as: Gria1 (glutamate receptor, ionotropic, AMPA1 (alpha 1)), Grik1 (glutamate receptor, ionotropic, kainate 1), Gsk3b (glycogen synthase kinase 3 beta) and Arnt1 aryl hydrocarbon receptor nuclear translocator-like. Conversely, some of the top blood biomarkers identified herein do not appear to have reliable human postmortem brain evidence to date: Btg1 (B-cell translocation gene 1, anti-proliferative), Ednrb (endothelin receptor type B), Elovl5 (ELOVL family member 5, elongation of long chain fatty acids), and Trpc1 (transient receptor potential cation channel, subfamily C, member 1).

A plurality of high probability blood candidate biomarker genes for mood state is identified. In an aspect, a select panel of biomarkers include for example, five genes involved in myelination (Mbp, Edg2, Mag, Pmp22 and Ugt8), six genes involved in growth factor signaling (Fgfr1, Fzd3, Erbb3, Igfbp4, Igfbp6), one gene involved in light transduction (PDE6D), and one gene involved in neurofilaments (Nefh). These genes have evidence of differential expression in human postmortem brains from mood disorder patients.

A predictive score developed based on a panel of 10 top candidate biomarkers, designated herein as BioM-10 (5 for high mood, 5 for low mood) shows specificity and sensitivity for high mood and low mood states.

A parallel profiling of cognitive and affective state was performed to investigate: (i) relationships between phenotypic items (“phenes”), including with objective motor measures, and (ii) relationships between subjects. This approach is useful in advancing current diagnostic classifications, and indicates that a combinatorial building-block structure underlies many psychiatric syndromes. The adaptation of microarray-based informatic tools for phenotypic analysis facilitates direct integration with gene expression profiling of blood in the same individuals, a strategy for molecular biomarker identification. Empirically derived clusterings of (endo)phenotypes and of patients provide a basis for genetic, pharmacological, and imaging research, as well as clinical practice.

In an aspect, some of the candidate genes included in a panel of biomarkers used herein, have no previous evidence for involvement in mood disorders other than being mapped to bipolar genetic linkage loci (Table 3). These genes constitute novel candidate genes for bipolar disorder and depression. The composition of biomarker panels for mood can be refined or changed for different sub-populations. Panels containing different number of biomarkers and different biomarkers can be developed using the guidelines described herein and from the complete list of more than 600 biomarkers identified (Tables 3 and 7).

Any number of biomarkers can be used as a panel for diagnosis. The panel may contain equal number of biomarkers for high and low mood, or different number of biomarkers associated with low mood than high mood. The panel may be tested as a microarray or as any form of diagnostic analysis.

In the present disclosure, gene expression changes in specific brain regions and blood of animal models developed were studied to identify one or more of the biomarkers disclosed herein. Data were obtained from a pharmacogenomic mouse model of bipolar (involving treatments with a stimulant—methamphetamine, and a mood stabilizer—valproate) as a discovery engine and cross-validator for the identification of potential peripheral blood biomarkers (see Ogden et al., (2004), Candidate genes, pathways and mechanisms for bipolar (manic-depressive) and related disorders: an expanded convergent functional genomics approach. Mol Psychiatry 9(11): 1007-29.). Data from other animal models of bipolar disorder, such as genetic models, can be used (see Le-Niculescu et al. (2008) Phenomic, convergent functional genomic and biomarker studies in a stress-reactive genetic animal model of bipolar disorder and co-morbid alcoholism. American Journal of Medical Genetics Part B (Neuropsychiatric Genetics) 147(2):134-66.

In an embodiment, a comprehensive analysis of: (i) fresh human blood gene expression data tied to illness state (quantitative measures of symptoms), (ii) cross-validation of blood gene expression profiling in conjunction with brain gene expression studies in animal models presenting key features of bipolar disorder, and (iii) integration of the results in the context of the available human genetic linkage/association and postmortem brain findings in the field is provided.

In an aspect, human blood gene expression studies were carried out in a primary group of bipolar subjects with low mood states and high mood states, as well as in a group of subjects with psychotic disorders (schizoaffective disorder, schizophrenia, and substance induced psychotic disorder), and in a second, independent, group of subjects with bipolar disorder. Genes that were differentially expressed in low mood vs. high mood subjects were compared with (i) the results of animal model data, (ii) human genetic linkage/association data and (iii) human postmortem brain data to cross-validate the results, prioritizing the genes, and identifying a short list of high probability candidate biomarker genes. A panel of candidate biomarker genes identified by this approach was then used to generate a prediction score for mood state (low mood/depression vs. high mood/mania). This prediction score was compared to the actual self-reported mood scores from human subjects. The prediction score developed by the analysis of convergent data provided a highly correlative basis for the diagnosis of mood state.

In an embodiment, a panel of biomarkers illustrated in Table 3 is suitable. These biomarkers include Mbp, Edg2, Fgfr1, Fzd3, Mag, Pmp22, Ugt8, Erbb3, Igfbp4, Igfbp6, Pde6d, Ptprm, Nefh, Atp2c1, Atxn1, Btg1, C6orf182, Dicer1, Dnajc6, Ednrb, Elovl5, Gnal, Klf5, Lin7a, Manea, Nupl1, Pde6b, Slc25a23, Synpo, Tgm2, Tjp3, Tpd52, Trpc1, Bclaf1, Gosr2, Rdx, Wdr34, Bic, C8orf42, Dock9, Hrasls, Ibrdc2, P2ry12, Specc1, Vil2.

In an embodiment, a panel of about 10 biomarkers, e.g., Mbp, Edg2, Fgfr1, Fzd3, Mag, Pmp22, Ugt8, Erbb3, Igfbp4, and Igfbp6, is suitable for diagnosing or predicting mood disorder.

In an embodiment, a panel of biomarkers include for example, Mbp, Edg2, Fzd3, Atxn1, and Ednrb that are increased in high mood (mania) condition.

An embodiment of a first sub-group of markers that are used for analysis include for example: Mbp, Edg2, Fzd3, Atxn1, Ednrb (markers for high mood) and Fgfr1, Mag, Pmp22, Ugt8, Erbb3 (markers for low mood). An embodiment includes a second sub-group e.g., Pde9a, Plxnd1, Camk2d, Dio2, Lepr (markers for high mood) and Igfbp4, Igfbp6, Pde6d, Ptprm, Nefh Atp2c1 (markers for low mood). An embodiment includes a third sub-group e.g., Myom2, Nfix, Nt5m, Or7e104p, Rrp1 (markers for high mood) and Atp2c1, Btg1, Elov5, Lrrc8b, Dicer1, Dnajc6 (markers for low mood). An embodiment includes a fourth sub-group e.g., Sept2, Sfrs4, Sla2, Tex261, Ube2i (markers for high mood) and Gnal, Klf5, Lin7a, Manea, Nupl1 (markers for low mood). An embodiment includes a fifth sub-group e.g., Usp7, Zdhhc4, Znf169, Cuedc1, Bivm (markers for high mood) and Pde6b, Slc25a23, Synpo, Tgm2, Tjp3 (markers for low mood). An embodiment includes a sixth sub-group e.g., Hla-dqa1, C20orf94, C21orf56, Flj10986, Loc91431 (markers for high mood), Tpd52, Trpc1, Phlda1, Znf502, Amn (markers for low mood) or a combination of one or more of the sub-groups 1-6 disclosed herein. Sub-groups 1-5 constitute a representative example and any number of sub-groups that has about 5, 10, 15, 20, 25, 30, 35, 40, 45, 50, 55, 60, 65, 70, 75, 80, 85, 90, 95, 100, 200, 300, 400, 500, 600, or more markers selected from Table 7.

An embodiment of a first sub-group of markers that are used for analysis include for example: Mbp, Edg2, Fzd3, Atxn1, Ednrb (markers for high mood), Fgfr1, Mag, Pmp22, Ugt8, Erbb3 (markers for low mood); second subgroup includes for example: Pde9a, Plxnd1, Camk2d, Dio2, Lepr (markers for high mood), Igfbp4, Igfbp6, Pde6d, Ptprm, Nefh, (markers for low mood); third subgroup includes for example: Myom2, Nfix, Nt5m, Or7e104p, Rrp1 (markers for high mood), Btg1, Elov5, Lrrc8b, Dicer1, Atp2c1, (markers for low mood); fourth subgroup includes for example: Sept2, Sfrs4, Sla2, Tex261, Ube2i (markers for high mood), Gnal, Klf5, Lin7a, Manea, Dnajc6 (markers for low mood); fifth subgroup includes for example: Usp7, Zdhhc4, Znf169, Cuedc1, Bivm (markers for high mood), Pde6b, Slc25a23, Synpo, Tgm2, Nupl1 (markers for low mood); and sixty subgroup includes for example: Hla-dqa1, C20orf94, C21orf56, Vil2, Loc91431 (markers for high mood), Tpd52, Trpc1, Phlda1, Tjp3, Amn (markers for low mood) or a combination of one or more of the sub-groups 1-6 disclosed herein. Sub-groups 1-5 constitute a representative example and any number of sub-groups that has about 5, 10, 15, 20, 25, 30, 35, 40, 45, 50, 55, 60, 65, 70, 75, 80, 85, 90, 95, 100, 200, 300, 400, 500, 600, or more markers selected from Tables 3 and 7.

A panel of 36 biomarkers, as illustrated in an example described herein, is a suitable subset that is useful in diagnosing a mood disorder. Larger subsets that includes for example, 150, 200, 250, 300, 350, 400, 450, 500, 600 or about 700 markers are also suitable. Smaller subsets that include high-value markers including about 2, 5, 10, 15, 20, 25, 50, 75, and 100 are also suitable. A variable quantitative scoring scheme can be designed using any standard algorithm, such as a variable selection or a subset feature selection algorithms can be used. Both statistical and machine learning algorithms are suitable in devising a frame work to identify, rank, and analyze association between marker data and phenotypic data (e.g., mood disorders).

In an embodiment, a prediction score for each subject is derived based on the presence or absence of e.g., 10 biomarkers of the panel in their blood. Each of the 10 biomarkers gets a score of 1 if it is detected as “present” (P) in the blood form that subject, 0.5 if it is detected as “marginally present” (M), and 0 if it is called “absent” (A). The ratio of the sum of the high mood biomarker scores divided by the sum of the low mood biomarker scores is multiplied by 100, and provides a prediction score. If the ratio of high biomarker genes to low mood biomarker genes is 1, i.e. the two sets of genes are equally represented, the mood prediction score is 1×100=100. The higher this score, the higher the predicted likelihood that the subject will have high mood. The predictive score was compared with actual self-reported mood scores in a primary cohort of subjects with a diagnosis of bipolar mood disorder. A prediction score of 100 and above had a 84.6% sensitivity and a 68.8% specificity for predicting high mood. A prediction score below 100 had a 76.9% sensitivity and 81.3% specificity for predicting low mood. The term “present” indicates that a particular biomarker is expressed to a detectable level, as determined by the technique used. For example, in an experiment involving a microarray or gene chip obtained from a commercial vendor Affymetrix (Santa Clara, Calif.), the embedded software rendered a “present” call for that biomarker. The term “present” refers to a detectable presence of the transcript or its translated protein/peptide and not necessarily reflects a relative comparison to for example, a sample from a normal subject. In other words, the mere presence or absence of a biomarker is assigned a value, e.g., 1 and a prediction score is calculated as described herein. The term “marginally present: refers to border line expression level that may be less intense than the “present” but statistically different from being marked as “absent” (above background noise), as determined by the methodology used.

In an embodiment, a prediction score based on differential expression (instead of “present”, “absent”) is used. For example, if a subject has a plurality of markers for high or low mood are differentially expressed, a prediction based on the differential expression of markers is determined. Differential expression of about 1.2 fold or 1.3 or 1.5 or 2 or 3 or 4 or 5-fold or higher for either increased or decreased levels can be used. Any standard statistical tool such as ANOVA is suitable for analysis of differential expression and association with high or low mood diagnosis or prediction.

A prediction based on the analysis of either high or low mood markers alone (instead of a ratio of high versus low mood markers) may also be practiced. If a plurality of high mood markers (e.g., about 6 out of 10 tested) are differentially expressed to a higher level compared to the low mood markers (e.g., 4 out of 10 tested), then a prediction or diagnosis of high mood status can be made by analyzing the expression levels of the high mood markers alone without factoring the expression levels of the low mood markers as a ratio.

In an embodiment, a detection algorithm uses probe pair intensities to generate a detection p-value and assign a Present, Marginal, or Absent call. Each probe pair in a probe set is considered as having a potential vote in determining whether the measured transcript is detected (Present) or not detected (Absent). The vote is described by a value called the Discrimination score [R]. The score is calculated for each probe pair and is compared to a predefined threshold Tau. Probe pairs with scores higher than Tau vote for the presence of the transcript. Probe pairs with scores lower than Tau vote for the absence of the transcript. The voting result is summarized as a p-value. The greater the number of discrimination scores calculated for a given probe set that are above Tau, the smaller the p-value and the more likely the given transcript is truly Present in the sample. The p-value associated with this test reflects the confidence of the Detection call.

Regarding detection p-value, a two-step procedure determines the Detection p-value for a given probe set. The Discrimination score [R] is calculated for each probe pair and the discrimination scores are tested against the user-definable threshold Tau. The detection Algorithm assesses probe pair saturation, calculates a Detection p-value, and assigns a Present, Marginal, or Absent call. In an embodiment, the default thresholds of the Affymetrix MAS 5 software were used.

In spiking experiments by the manufacturer to establish default thresholds (adding of known quantities of test transcripts to a mixture, to measure the sensitivity of the Affymetrix MAS 5 detection algorithm) 80% of spiked transcripts are called Present at a concentration of 1.5 pM. This concentration corresponds to approximately one transcript in 100,000 or 3.5 copies per cell. The false positive rate of making a Present call was roughly 10%, as noted by 90% of the transcripts being called Absent when not spiked into the sample (0 pM concentration).

The term “predictive” or the term “prognostic” does not imply 100% predictive ability. The use of these terms indicates that subjects with certain characteristics are more likely to experience a particular mood state or clinical outcome than subjects who do not have such characteristics. For example, characteristics that determine the prediction include one or more of the biomarkers for the mood disorder disclosed herein. The phrase “clinical outcome” refers to biological or biochemical or physical or physiological responses to treatments or therapeutic agents that are generally prescribed for that condition compared to a condition would occur in the absence of any treatment. A “clinically positive outcome” does not necessarily indicate a cure, but could indicate a lessening of symptoms experienced by a subject.

The terms “marker” and “biomarker” are synonymous and as used herein, refer to the presence or absence or the levels of nucleic acid sequences or proteins or polypeptides or fragments thereof to be used for associating or correlating a phenotypic state. A biomarker includes any indicia of the level of expression of an indicated marker gene. The indicia can be direct or indirect and measure over- or under-expression of the gene given the physiologic parameters and in comparison to an internal control, normal tissue or another phenotype. Nucleic acids or proteins or polypeptides or portions thereof used as markers are contemplated to include any fragments thereof, in particular, fragments that can specifically hybridize with their intended targets under stringent conditions and immunologically detectable fragments. One or more markers may be related. Marker may also refer to a gene or DNA sequence having a known location on a chromosome and associated with a particular gene or trait. Genetic markers associated with certain diseases or for pre-disposing disease states can be detected in the blood and used to determine whether an individual is at risk for developing a disease. Levels of gene expression and protein levels are quantifiable and the variation in quantification or the mere presence or absence of the expression may also serve as markers. Using proteins/peptides as biomarkers can include any method known in the art including, without limitation, measuring amount, activity, modifications such as glycosylation, phosphorylation, ADP-ribosylation, ubiquitination, etc., immunohistochemistry (IHC).

As used herein, “array” or “microarray” refers to an array of distinct polynucleotides, oligonucleotides, polypeptides, or oligopeptides synthesized on a substrate, such as paper, nylon, or other type of membrane, filter, chip, glass slide, or any other suitable solid support. Arrays also include a plurality of antibodies immobilized on a support for detecting specific protein products. There are several microarrays that are commercially available. A microarray may include one or more biomarkers disclosed herein. A panel of about 20 biomarkers as nucleic acid fragments can be included in an array. The nucleic acid fragments may include oligonucleotides or amplified partial or complete nucleotide sequences of the biomarkers. The term “consisting essentially of” generally refers to a collection of markers that substantially affects the determination of the disorder and may include other components such as controls. For example, a microarray consists essentially of markers from Table 3.

In an embodiment, the microarray is prepared and used according to the methods described in U.S. Pat. No. 5,837,832, Chee et al.; PCT application WO95/11995, Chee et al.; Lockhart et al., 1996. Nat Biotech., 14:1675-80; and Schena et al., 1996. Proc. Natl. Acad. Sci. 93:10614-619, all of which are herein incorporated by reference to the extent they relate to methods of making a microarray. Arrays can also be produced by the methods described in Brown et al., U.S. Pat. No. 5,807,522. Arrays and microarrays may be referred to as “DNA chips” or “protein chips.”

A variety of clustering methods are available for microarray-based gene expression analysis. See for example, Shamir & Sharan (2002) Algorithmic approaches to clustering gene expression data. In Current Topics In Computational Molecular Biology (Edited by: Jiang T, Xu Y, Smith T). 2002, 269-300; Tamames et al., (2002): Bioinformatics methods for the analysis of expression arrays: data clustering and information extraction, J Biotechnol, 98:269-283.

“Therapeutic agent” means any agent or compound useful in the treatment, prevention or inhibition of mood disorder or a mood-related disorder.

The term “condition” refers to any disease, disorder or any biological or physiological effect that produces unwanted biological effects in a subject.

The term “subject” refers to an animal, or to one or more cells derived from an animal. The animal may be a mammal including humans. Cells may be in any form, including but not limited to cells retained in tissue, cell clusters, immortalized cells, transfected or transformed cells, and cells derived from an animal that have been physically or phenotypically altered.

Any body fluid of an animal can be used in the methods of the invention. Suitable body fluids include a blood sample (e.g., whole blood, serum or plasma), urine, saliva, cerebrospinal fluid, tears, semen, and vaginal secretions. Also, lavages, tissue homogenates and cell lysates can be utilized.

Many different methods can be used to determine the levels of markers. For example, protein arrays, protein chips, cDNA microarrays or RNA microarrays are suitable. More specifically, one of ordinary skill in the art will appreciate that in one example, a microarray may comprise the nucleic acid sequences representing genes listed in Table 1. For example, functionality, expression and activity levels may be determined by immunohistochemistry, a staining method based on immunoenzymatic reactions uses monoclonal or polyclonal antibodies to detect cells or specific proteins. Typically, immunohistochemistry protocols include detection systems that make the presence of markers visible (to either the human eye or an automated scanning system), for qualitative or quantitative analyses. Mass-spectrometry, chromatography, real-time PCR, quantitative PCR, probe hybridization, or any other analytical method to determine expression levels or protein levels of the markers are suitable. Such analysis can be quantitative and may also be performed in a high-through put fashion. Cellular imaging systems are commercially available that combine conventional light microscopes with digital image processing systems to perform quantitative analysis on cells and tissues, including immunostained samples. (See e.g. the CAS-200 System (Becton, Dickinson & Co.)). Some other examples of methods that can be used to determine the levels of markers include immunohistochemistry, automated systems, quantitative IHC, semi-quantitative IHC and manual methods. Other analytical systems include western blotting, immunoprecipitation, fluorescence in situ hybridization (FISH), and enzyme immunoassays.

The term “diagnosis”, as used in this specification refers to evaluating the type of disease or condition from a set of marker values and/or patient symptoms where the subject is suspected of having a disorder. This is in contrast to disease predisposition, which relates to predicting the occurrence of disease before it occurs, and the term “prognosis”, which is predicting disease progression in the future based on the marker levels/patterns.

The term “correlating,” as used in this specification refers to a process by which one or more biomarkers are associated to a particular disease state, e.g., mood disorder. In general, identifying such correlation or association involves conducting analyses that establish a statistically significant association- and/or a statistically significant correlation between the presence (or a particular level) of a marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed.

This relationship or association can be determined by comparing biomarker levels in a subject to levels obtained from a control population, e.g., positive control—diseased (with symptoms) population and negative control—disease-free (symptom-free) population. The biomarkers disclosed herein provide a statistically significant correlation to diagnosis at varying levels of probability. Subsets of markers, for example a panel of about 20 markers, each at a certain level range which are a simple threshold, are said to be correlative or associative with one of the disease states. Such a panel of correlated markers can be then be used for disease detection, diagnosis, prognosis and/or treatment outcome. Preferred methods of correlating markers is by performing marker selection by any appropriate scoring method or by using a standard feature selection algorithm and classification by known mapping functions. A suitable probability level is a 5% chance, a 10% chance, a 20% chance, a 25% chance, a 30% chance, a 40% chance, a 50% chance, a 60% chance, a 70% chance, a 75% chance, a 80% chance, a 90% chance, a 95% chance, and a 100% chance. Each of these values of probability is plus or minus 2% or less. A suitable threshold level for markers of the present invention is about 25 pg/mL, about 50 pg/mL, about 75 pg/mL, about 100 pg/mL, about 150 pg/mL, about 200 pg/mL, about 400 pg/mL, about 500 pg/mL, about 750 pg/mL, about 1000 pg/mL, and about 2500 pg/mL.

Prognosis methods disclosed herein that improve the outcome of a disease by reducing the increased disposition for an adverse outcome associated with the diagnosis. Such methods may also be used to screen pharmacological compounds for agents capable of improving the patient's prognosis, e.g., test agents for mood disorders.

The analysis of a plurality of markers, for example, a panel of about 20 or 10 markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples may allow the identification of changes in marker levels over time, within a period of interest, or in response to a certain treatment.

In another embodiment, a kit for the analysis of markers includes for example, devises and reagents for the analysis of at least one test sample and instructions for performing the assay. Optionally, the kits may contain one or more means for using information obtained from marker assays performed for a marker panel to diagnose mood disorders. Probes for markers, marker antibodies or antigens may be incorporated into diagnostic assay kits depending upon which markers are being measured. A plurality of probes may be placed in to separate containers, or alternatively, a chip may contain immobilized probes. In an embodiment, another container may include a composition that includes an antigen or antibody preparation. Both antibody and antigen preparations may preferably be provided in a suitable titrated form, with antigen concentrations and/or antibody titers given for easy reference in quantitative applications.

The kits may also include a detection reagent or label for the detection of specific reaction between the probes provided in the array or the antibody in the preparation for immunodetection. Suitable detection reagents are well known in the art as exemplified by fluorescent, radioactive, enzymatic or otherwise chromogenic ligands, which are typically employed in association with the nucleic acid, antigen and/or antibody, or in association with a secondary antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like.

In some embodiments, the methods of correlating biomarkers with treatment regimens can be carried out using a computer database. Computer-assisted methods of identifying a proposed treatment for mood disorders are suitable. The method involves the steps of (a) storing a database of biological data for a plurality of patients, the biological data that is being stored including for each of said plurality of patients (i) a treatment type, (ii) at least one marker associated with a mood disorder and (iii) at least one disease progression measure for the mood disorder from which treatment efficacy can be determined; and then (b) querying the database to determine the dependence on the marker of the effectiveness of a treatment type in treating the mood disorder, to thereby identify a proposed treatment as an effective treatment for a subject carrying the marker correlated with the mood disorder.

In an embodiment, treatment information for a patient is entered into the database (through any suitable means such as a window or text interface), marker information for that patient is entered into the database, and disease progression information is entered into the database. These steps are then repeated until the desired number of patients has been entered into the database. The database can then be queried to determine whether a particular treatment is effective for patients carrying a particular marker, not effective for patients carrying a particular marker, and the like. Such querying can be carried out prospectively or retrospectively on the database by any suitable means, but is generally done by statistical analysis in accordance with known techniques, as described herein.

### EXAMPLES

The following examples are to be considered as exemplary and not restrictive or limiting in character and that all changes and modifications that come within the spirit of the disclosure are desired to be protected.

### Example 1

### Experimental Framework for Identification of Biomarkers Used in Diagnosis of Mood Disorders

Gene expression changes in specific brain regions and blood from a pharmacogenomic animal model were used as cross-validators for identification of potential human blood biomarkers. Pharmacogenomic mouse model of relevance to bipolar disorder consists of treatments with an agonist of the illness/bipolar disorder-mimicking drug (methamphetamine) and an antagonist of the illness/bipolar disorder-treating drug (valproate). The pharmacogenomic approach is a tool for tagging genes that may have pathophysiological relevance.

Human blood gene expression studies were carried out in a cohort of bipolar subjects. Genes that were differentially expressed in low mood vs. high mood subjects were compared with: 1) the results of the animal model brain and blood data, as well as 2) published human genetic linkage/association data, and 3) human postmortem brain data, as a way of cross-validating the findings, prioritizing them, and coming up with a short list of high probability candidate biomarker genes (FIGS. 2A and 3).

A Visual-Analog Scale (VAS) for mood was used for the scoring analysis. This approach avoids the issue of corrections for multiple comparisons that would arise if multiple symptom (phenotypic) scores (i.e. “phenes”) were analyzed in a comprehensive phenotypic battery changed in relationship with all genes on a GeneChip microarray. Larger sample cohorts would be needed for the latter approach.

A panel of top candidate biomarker genes for mood state identified was then used to generate a prediction score for mood state (low mood vs. high mood). This prediction score was compared to the actual self-reported mood scores from bipolar subjects (FIG. 4). This panel of mood biomarkers and prediction score were examined in a separate independent cohort of psychotic disorders patients for which gene expression data and mood state data is available (FIG. 5), and in a second independent cohort of bipolar disorder subjects (FIG. 6).

Sample size for human subjects (n=29 for the bipolar cohort, n=30 for the psychotic disorders cohort) is comparable to the size of cohorts for human postmortem brain gene expression studies. Live donor blood samples were used instead of postmortem donor brains, with the advantage of better phenotypic characterization, more quantitative state information, and less technical variability.

For the animal model work, isogenic mouse strain was used. Three independent de novo biological experiments were performed, at different times, with different batches of mice. This overall design is geared to factor out both biological and technical variability. Concordance between reproducible microarray experiments using the latest generations of oligonucleotide microarrays and other methodologies such as quantitative PCR, with their own attendant technical limitations, is estimated to be over 90%. For the human blood samples gene expression analyses, which are the results of single biological experiments, a very restrictive, all or nothing induction of gene expression (change from Absent Call to Present Call). It is possible that not all biomarker genes for mood may show this complete induction related to state, but rather some may show modulation in gene expression levels, and are thus missed by this filtering. Moreover, given the genetic heterogeneity and variable environmental exposure, it is possible, indeed likely, that not all subjects may show changes in all the biomarker genes. Hence two stringency thresholds were used: changes in 75% of subjects, and in 60% of subjects with low mood vs. high mood. This approach is predicated on the existence of multiple cross-validators for each gene that is called a candidate biomarker (FIG. 2B): 1) is it changed in human blood, 2) is it changed in animal model brain, 3) is it changed in animal model blood, 4) is it changed in postmortem human brain, and 5) does it map to a human genetic linkage locus. All these lines of evidence are the result of independent experiments.

Human blood gene expression changes may be influenced by the presence or absence of both medications and drugs of abuse. That medications and drugs of abuse may have effects on mood state and gene expression is in fact being partially modeled in the mouse pharmacogenomic model, with valproate and methamphetamine treatments respectively. It is the association of blood biomarkers with mood state that is the primary purpose of this analysis, regardless of the proximal causes, which could be diverse (see FIG. 2B).

The human subjects used in this example included those who were directly recruited, and data collected in other procedures/settings. Blood samples were collected.

Human data from three independent cohorts of patients are presented. One cohort consists of 29 subjects with bipolar I disorder. The second cohort consists of 30 subjects with psychotic disorders (schizophrenia, schizoaffective disorder, and substance induced psychosis). The third cohort consists of 19 subjects with bipolar I disorder. The diagnosis is established by a structured clinical interview—Diagnostic Interview for Genetic Studies (DIGS), which has details on the course of illness and phenomenology, and is the scale used by the Genetics Initiative Consortia for both Bipolar Disorder and Schizophrenia.

Subjects included men and women over 18 years of age. A demographic breakdown is shown in Table 1. Initial studies were focused primarily on a male population, due to the demographics of the catchment area (primarily male in a VA Medical Center), and to minimize any potential gender-related state effects on gene expression, which would have decreased the discriminative power of the analysis for the sample size used. Subjects were recruited from the general population, the patient population at the IU school of Medicine, the Indianapolis VA Medical Center, as well as various facilities that serve people with mental illnesses in Indiana. The subjects were recruited largely through referrals from care providers, the use of brochures left in plain sight in public places and mental health clinics, and through word of mouth. Subjects were excluded if they had significant medical or neurological illness or had evidence of active substance abuse or dependence. All subjects understood and signed informed consent forms before assessments began. All subjects signed an informed consent form detailing the research goals, procedure, caveats and safeguards. Subjects completed diagnostic assessments (DIGS), and then a visual-analog scale for mood (VAS Mood) at the time of blood draw.

Human Blood Gene Expression Experiments and Analysis:

RNA extraction: 2.5-5 ml of whole blood was collected into each PaxGene tube by routine venipuncture. PaxGene tubes contain proprietary reagents for the stabilization of RNA. The cells from whole blood will be concentrated by centrifugation, the pellet washed, resuspended and incubated in buffers containing Proteinase K for protein digestion. A second centrifugation step will be done to remove residual cell debris. After the addition of ethanol for an optimal binding condition the lysate is applied to a silica-gel membrane/column. The RNA bound to the membrane as the column is centrifuged and contaminants are removed in three wash steps. The RNA is then eluted using DEPC-treated water.

Globin reduction: To remove globin mRNA, total RNA from whole blood is mixed with a biotinylated Capture Oligo Mix that is specific for human globin mRNA. The mixture is then incubated for 15 min to allow the biotinylated oligonucleotides to hybridize with the globin mRNA. Streptavidin Magnetic Beads are then added, and the mixture is incubated for 30 min. During this incubation, streptavidin binds the biotinylated oligonucleotides, thereby capturing the globin mRNA on the magnetic beads. The Streptavidin Magnetic Beads are then pulled to the side of the tube with a magnet, and the RNA, depleted of the globin mRNA, is transferred to a fresh tube. The treated RNA is further purified using a rapid magnetic bead-based purification method. This consists of adding an RNA Binding Bead suspension to the samples, and using magnetic capture to wash and elute the GLOBINclear RNA.

Sample Labeling: Sample labeling is performed using the Ambion MessageAmp II-BiotinEnhanced aRNA amplification kit. The procedure is briefly outlined herein and involves the following steps:

1. Reverse Transcription to Synthesize First Strand cDNA is primed with the T7 Oligo(dT) Primer to synthesize cDNA containing a T7 promoter sequence.

2. Second Strand cDNA Synthesis converts the single-stranded cDNA into a double-stranded DNA (dsDNA) template for transcription. The reaction employs DNA Polymerase and RNase H to simultaneously degrade the RNA and synthesize second strand cDNA.

3. cDNA Purification removes RNA, primers, enzymes, and salts that would inhibit in vitro transcription.

4. In Vitro Transcription to Synthesize aRNA with Biotin-NTP Mix generates multiple copies of biotin-modified aRNA from the double-stranded cDNA templates; this is the amplification step.

5. aRNA Purification removes unincorporated NTPs, salts, enzymes, and inorganic phosphate to improve the stability of the biotin-modified aRNA.

Microarrays: Biotin labeled aRNA are hybridized to Affymetrix HG-U133 Plus 2.0 GeneChips according to manufacturer's protocols (Affymetrix Inc., Santa Clara, Calif.). All GAPDH 3′/5′ ratios should be less than 2.0 and backgrounds under 50. Arrays are stained using standard Affymetrix protocols for antibody signal amplification and scanned on an Affymetrix GeneArray 2500 scanner with a target intensity set at 250. Present/Absent calls are determined using GCOS software with thresholds set at default values.

The human blood gene expression experiments and analysis was performed at two levels: (i) high threshold >75%; 3× enrichment, and (ii) low threshold (>60%; 1.5× enrichment). The animal model data included pharmacogenomic models that involved DBP KO mouse.

The cross-validation and integration of data from human blood gene expression, mouse models and other mouse and human data were processed through a convergent functional genomics approach.

For generating the animal model data, standard pharmacogenomic testing methodologies were adopted. All experiments were performed with male C57/BL6 mice, 8 to 12 weeks of age, obtained from Jackson Laboratories (Bar Harbor, Me.), and acclimated for at least two weeks in an animal facility prior to any experimental manipulation. The bipolar pharmacogenomic model included Methamphetamine and Valproate treatments in mice (see Ogden et al. (2004)). Briefly, mice were treated by intraperitoneal injection with either single-dose saline, methamphetamine (10 mg/kg), valproate (200 mg/kg), or a combination of methamphetamine and valproate (10 mg/kg and 200 mg/kg respectively). Three independent de novo biological experiments were performed at different times. Each experiment included three mice per treatment condition, for a total of 9 mice per condition across the three experiments.

RNA extraction and microarray analysis: Standard techniques were used to obtain total RNA (22 gauge syringe homogenization in RLT buffer) and to purify the RNA (RNeasy mini kit, Qiagen, Valencia, Calif.) from micro-dissected mouse brain regions. For the human and whole mouse blood RNA extraction, PAXgene blood RNA extraction kit (PreAnalytiX, a QIAGEN/BD company) was used, followed by GLOBINclear™-Human or GLOBINclear™-Mouse/Rat (Ambion/Applied Biosystems Inc., Austin, Tex.) to remove the globin mRNA. All the methods and procedures were carried out as per manufacturer's instructions. The quality of the total RNA was confirmed using an Agilent 2100 Bioanalyzer (Agilent Technologies, Palo Alto, Calif.). The quantity and quality of total RNA was also independently assessed by 260 nm UV absorption and by 260/280 ratios, respectively (Nanodrop spectrophotometer). Starting material of total RNA labeling reactions was kept consistent within each independent microarray experiment.

For the mouse analysis, blood or brain tissue regions from 3 mice were pooled for each experimental condition, and equal amounts of total RNA extracted from tissue samples or blood was used for labeling and microarray assays. Mouse Genome 430 2.0 arrays (Affymetrix, Santa Clara, Calif.) were used. The GeneChip™ Mouse Genome 430 2.0 Array contain over 45,000 probe sets that analyze the expression level of over 39,000 transcripts and variants from over 34,000 well-characterized mouse genes. For the human analysis, Affymetrix Human Genome U133 Plus 2.0 GeneChip with over 40,000 genes and ESTs were used. Standard manufacturer's protocols were used to reverse transcribe the messenger RNA and generate biotinlylated cRNA. The amount of cRNA used to prepare the hybridization cocktail was kept constant intra-experiment. Samples were hybridized at 45° C. for 17 hours under constant rotation. Arrays were washed and stained using the Affymetrix Fluidics Station 400 and scanned using the Affymetrix Model 3000 Scanner controlled by GCOS software. All sample labeling, hybridization, staining and scanning procedures were carried out as per manufacturer's recommendations.

Quality control: All arrays were scaled to a target intensity of 1000 using Affymetrix MASv 5.0 array analysis software. Quality control measures including 3′/5′ ratios for GAPDH and beta-actin, scaling factors, background, and Q values were within acceptable limits.

Microarray data analysis: Data analysis was performed using Affymetrix Microarray Suite 5.0 software (MAS v5.0). Default settings were used to define transcripts as present (P), marginal (M), or absent (A). For the pharmacogenomic mouse model, a comparison analysis was performed for each drug treatment, using its corresponding saline treatment as the baseline. “Signal,” “Detection,” “Signal Log Ratio,” “Change,” and “Change p-value,” were obtained from this analysis. Only transcripts that were called Present in at least one of the two samples (saline or drug) intra-experiment, and that were reproducibly changed in the same direction in at least two out of three independent experiments, were analyzed further. For the DBP knock-out mice, a comparison analysis was performed for each KO saline and KO Meth mouse, using WT saline mice as the baseline “Signal,” “Detection,” “Signal Log Ratio,” “Change,” and “Change p-value,” were obtained from this analysis. Only transcripts that were called Present in at least one of the two samples in a comparison pair, and that were reproducibly changed in the same direction in at least six out of 9 comparisons, were analyzed further.

### Example 2

### Analysis and Identification of Biomarkers

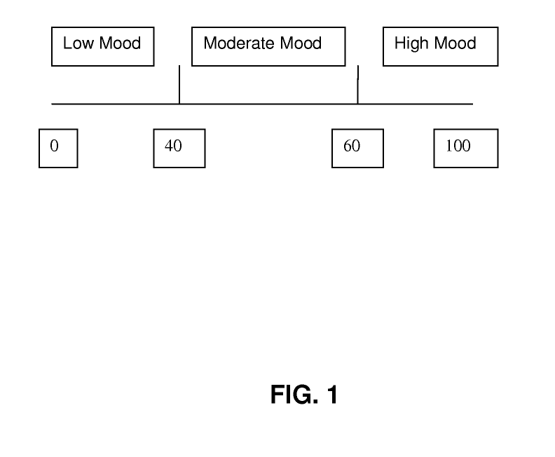

Gene expression profiling studies were performed with peripheral whole blood samples from a primary cohort of 29 human subjects with bipolar I disorder (27 males, 2 females) (Table 1). 13 had low self-reported mood scores (below 40) on the Visual-Analog (VAS) Mood Scale (FIG. 1), and 13 had high self-reported mood scores (above 60). 3 of them had intermediate mood scores (between 40 and 60). Their mood scores at time of blood collection were used as a way of narrowing the field and identifying candidate biomarker genes for mood. Only t all or nothing gene expression differences were identified by Absent (A) vs. Present (P) Calls in the Affymetrix MAS software. Genes whose expression is detected as Absent in the Low Mood subjects and detected as Present in the High Mood subjects were classified, as being candidate biomarker genes for elevated mood state (mania). Conversely, genes whose expression is detected as Present in the Low Mood subjects and Absent in the High Mood subjects are being classified as candidate biomarker genes for low mood state (depression) (Tables 2 and 3). It is possible that some of the genes associated with high mood state or low mood state may not necessarily be involved in the induction of that state, but rather in its suppression as part of a homeostatic regulatory networks or treatment response mechanisms (similar conceptually to oncogenes and tumor-suppressor genes).

Two thresholds for analysis of gene expression differences between low mood and high mood (Table 2) were undertaken. First, a high threshold was used, with at least 75% of subjects in a cohort showing a change in expression from Absent to Present between low and high mood (reflecting an at least 3 fold mood state related enrichment of the genes thus filtered) As psychiatric disorders are clinically and (likely) genetically heterogeneous, with different combinations of genes and biomarkers present in different subgroups, a low threshold was also used, with at least 60% of subjects in a cohort showing a change in expression from Absent to Present between low and high mood (reflecting an at least 1.5 fold mood state related enrichment of the genes thus filtered). The high threshold identified candidate biomarker genes that are more common for all subjects, with a lower risk of false positives, whereas the lower threshold identified genes that are present in more restricted subgroups of subjects, with a lower risk of false negatives. The high threshold candidate biomarker genes have, as an advantage, a higher degree of reliability, as they are present in at least 75% of all subjects with a certain mood state (high or low) tested. They may reflect common aspects related to mood disorders across a diverse subject population, but may also be a reflection of the effects of common medications used in the population tested, such as mood stabilizers. The low threshold genes may have lower reliability compared to the high threshold, being present in at least 60% of the subject population tested, but, nevertheless, captures more of the diversity of genes and biological mechanisms present in a genetically diverse human subject population.

By cross-validating with animal model and other human datasets (FIG. 2A) using CFG, a shorter list of genes was identified for which there are external corroborating line of evidence (e.g., human genetic evidence, human postmortem brain data, animal model brain and blood data) linking them to mood disorders (bipolar disorder, depression), thus reducing the risk of false positives. This cross-validation identifies candidate biomarkers that are more likely directly related to the relevant neuropathology, as opposed to being potential artifactual effects or indirect effects of lifestyle, environment, etc.

Using the approach described herein, out of over 40,000 genes and ESTs on the Affymetrix Human Genome U133 Plus 2.0 GeneChip, by using the high threshold, in an embodiment, about 21 novel candidate biomarker genes (13 genes with known functions and 7 ESTs) (Table 3), of which 8 had at least one line of prior independent evidence for potential involvement in mood disorders (i.e. CFG score of 3 or above). In addition to the high threshold genes, by using the low threshold, a larger list totaling 661 genes (539 genes and 122 ESTs) (Table 7), of which an additional 24 had at least two lines of prior independent evidence for potential involvement in mood disorders (i.e. CFG score of 3 or above). Of interest, four of the low threshold candidate biomarker genes (Bclaf1 and Rdx8, Gosr2 and Wdr3413) are changed in expression in the same direction, in lymphoblastoid cell lines (LCLs) from bipolar subjects.

Making a combined list of all the high value candidate biomarker genes identified as described above—including the high threshold genes with at least on other external line of evidence (8) and of the additional low threshold genes with at least two other external lines of evidence (24), and the low threshold genes with prior LCL evidence (4), a list of 36 candidate biomarker genes for mood, prioritized based on CFG score (Table 3) was developed.

In an embodiment, selecting the 5 top scoring candidate biomarkers for high mood (MBP, EDG2, FZD3, ATXN1, EDNRB) and the 5 top scoring candidate biomarkers for low mood (FGFR1, MAG, PMP22, UGT8, ERBB3), a panel of 10 biomarkers for mood disorder was developed that has diagnostic and predictive value.

To test the predictive value of a panel (e.g, the BioM-10 Mood panel), a cohort of 29 bipolar disorder subjects, containing the 26 subjects (13 low mood, 13 high mood) from which the candidate biomarker data was derived, as well as 3 additional subjects with mood in the intermediate range (self-reported mood scores between 40 and 60) was used. A prediction score for each subject, based on the presence or absence of the 10 biomarkers of the panel in the blood GeneChip data. Each of the 10 biomarkers gets a score of 1 if it is detected as Present (P) in the blood form that subject, 0.5 if it is detected as Marginally Present (M), and 0 if it is called Absent (A). The ratio of the sum of the high mood biomarker scores divided by the sum of the low mood biomarker scores is multiplied by 100, and provides a prediction score. If the ratio of high biomarker genes to low mood biomarker genes is 1, i.e. the two sets of genes are equally represented, the mood prediction score is 1×100=100. The higher this score, the higher the predicted likelihood that the subject will have high mood. The predictive score was compared with actual self-reported mood scores in the primary cohort of subjects with a diagnosis of bipolar mood disorder (n=29). A prediction score of 100 and above had a 84.6% sensitivity and a 68.8% specificity for predicting high mood. A prediction score below 100 had a 76.9% sensitivity and 81.3% specificity for predicting low mood (Table 4A and FIG. 4).

Table 5 shows a representative sample of biological roles based on ingenuity pathway analysis (IPA) of biological roles categories among the top blood candidate biomarker genes for mood.

Human blood gene expression analysis was conducted in an independent cohort consisting of 30 subjects with other psychotic disorders (schizophrenia, schizoaffective disorder, substance induced psychosis), who also had mood state scores obtained at the time of the blood draw. The subjects in the psychosis cohort also had a distribution of low (n=9), intermediate (n=7) and high (n=14) mood scores. This cohort was used as a way to verify the predictive power of the mood state biomarker panel, independent of a bipolar disorder diagnosis.

In the psychotic disorders cohort (n=30), with various psychotic disorders diagnoses, a prediction score of 100 and above had a 71.4% sensitivity and a 62.5% specificity for predicting high mood. A prediction score below 100 had a 66.7% sensitivity and 61.9% specificity for predicting low mood (Table 4B and FIG. 5).

Human blood gene expression analysis was also conducted in a second independent bipolar disorder cohort, subsequently collected, consisting of 19 subjects. The subjects in the secondary bipolar cohort had a distribution of low (n=6), intermediate (n=3) and high (n=10) mood scores. The second bipolar cohort was used as a replication cohort, to verify the predictive power of the mood state biomarker panel identified by analysis of data from the primary bipolar cohort.

In the second bipolar cohort (n=19), a prediction score of 100 and above had a 70.0% sensitivity and a 66.7% specificity for predicting high mood. A prediction score below 100 had a 66.7% sensitivity and 61.5% specificity for predicting low mood (Table 4C and FIG. 6).

The primary and secondary bipolar mood disorder cohorts are apriori more related and germane to mood state biomarkers identification, but may have blood gene expression changes due at least in part to the common pharmacological agents used to treat bipolar mood disorders. The psychotic disorders cohort may have blood gene expression changes related to mood state irrespective of the diagnosis and the different medication classes subjects with different diagnoses are on (Table 1 and FIG. 2B). The psychosis cohort was also notably different in terms of the ethnic distribution (see Table 1b).

The MIT/Broad Institute connectivity map was interrogated with a signature query composed of the genes in the BioM-10 Mood panel of top biomarkers for low mood and high mood (FIG. 5). The effects of drugs in the Connectivity Map database and their effects on gene expression as the effects of high mood or low mood on gene expression. As such, as part of the signature query, the 5 biomarkers for high mood were considered as genes “Increased” by high mood, the 5 biomarkers for low mood were genes “Decreased” by high mood. The analysis revealed that sodium phenylbutyrate exerts the most similar effects to high mood, and novobiocin the most similar effects to low mood. Conventional gene expression analysis may not result in the same set of biomarkers.

By selecting 5 candidate biomarkers for high mood and 5 candidate biomarkers for low mood, a panel of 10 biomarkers for mood disorder that has diagnostic and predictive value was developed based on the scores of the biomarkers for low and high mood sections.

Thus, the biomarkers identified herein provide quantitative tools for predicting disease states/conditions in subjects suspected of having a mood disorder or in any individual for psychiatric evaluation.

A meta-analysis of the two bipolar subject cohorts was also conducted. A panel of 10 top biomarkers identified by the meta-analysis was tested for sensitivity and specificity for low and high mood in the two bipolar cohorts (Table 4D). The panel included Edg2, Ednrb, Vil2, Bivm, Camk2d (high mood markers) and Trpc1, Elovl5, Ugt8, Btg1, Nefh (low mood markers). A number of new biomarker genes revealed only in the meta-analysis were identified (see Table 3).

### Example 3

### Cross-Validation and Integration Using Convergent Functional Genomics Approaches to Identify and Prioritize Biomarkers for Mood Disorders

The identities of transcripts were established using NetAFFX™ to correlate the GeneChip® array results with array design and annotation information (Affymetrix, Santa Clara, Calif.), and confirmed by cross-checking the target mRNA sequences that had been used for probe design in the Mouse Genome 430 2.0 Array GeneChip® or the Affymetrix Human Genome U133 Plus 2.0 GeneChip® with the GenBank database. Where possible, identities of ESTs were established by BLAST searches of the nucleotide database. A National Center for Biotechnology Information (NCBI) (Bethesda, Md.) BLAST analysis of the accession number of each probe-set was done to identify each gene name. BLAST analysis identified the closest (most similar) known gene existing in the database (the highest known gene at the top of the BLAST list of homologues) which then could be used to search the GeneCards database (Weizmann Institute, Rehovot, Israel). Probe-sets that did not have a known gene were labeled “EST” and their accession numbers were kept as identifiers.

Human Postmortem Brain Convergence: Information about the candidate genes was obtained using GeneCards, the Online Mendelian Inheritance of Man database at the NCBI database, as well as database searches using PubMed and various combinations of keywords (gene name, bipolar, depression, psychosis, schizophrenia, alcoholism, suicide, dementia, Alzheimer, opiates, cocaine, marijuana, hallucinogens, amphetamines, benzodiazepines, human, brain, postmortem, lymphocytes, fibroblasts). Postmortem convergence was deemed to occur for a gene (or a biomarker) if there were human postmortem data showing changes in expression of that gene in brains from patients with mood disorders (bipolar disorder, depression), or secondarily of other major neuropsychiatric disorders that impact mood (schizophrenia, anxiety, alcoholism).

Human Genetic Data Convergence: To designate convergence for a particular gene, the gene may have positive reports from candidate gene association studies, or map within 10 cM of a microsatellite marker for which at least one study demonstrated evidence for genetic linkage to mood disorders (bipolar disorder or depression) or secondarily to another neuropsychiatric disorder. The University of Southampton's sequence-based integrated map of the human genome (The Genetic Epidemiological Group, Human Genetics Division, was used to obtain cM locations for both genes and markers. The sex-averaged cM value was calculated and used to determine convergence to a particular marker. For markers that were not present in the Southampton database, the Marshfield database (Center for Medical Genetics, Marshfield, Wis., USA) was used with the NCBI Map Viewer web-site to evaluate linkage convergence.

Gene Ontology (GO) analysis: The NetAffx™ Gene Ontology Mining Tool (Affymetrix, Santa Clara, Calif.) was employed to categorize the genes in the datasets into functional categories, using the Biological Process ontology branch.

Ingenuity analysis: Ingenuity Pathway Analysis 5.1 software(Ingenuity Systems, Redwood City, Calif.) was used to analyze the direct interactions of the top candidate genes resulting from the CFG analysis, biological roles, as well as employed to identify genes in the datasets that are the target of existing drugs.

Convergent Functional Genomics (CFG) Analysis Scoring (see FIG. 2A) is presented as follows:

(i) Biomarkers were given the maximum score of 2 points if changed in the human blood samples with high threshold analysis, and only 1 point if changed with low threshold.

(ii) Biomarkers received 1 point for each external cross-validating line of evidence (human postmortem brain data, human genetic data, animal model brain data, and animal model blood data).

(iii) Biomarkers received additional bonus points if changed in human brain and blood, as follows:
(a) 2 points if changed in the same direction;
(b) 1 point if changed in opposite direction;

(iv) Biomarkers also received additional bonus points if changed in brain and blood of the animal model, as follows:
(a) 1 point if changed in the same direction in the brain and blood;
(b) 0.5 points if changed in opposite direction.

Thus the total maximum CFG score that a candidate biomarker gene can have is 9 (2+4+2+1). To identify blood biomarkers the scoring pattern described herein is biased more towards awarding additional points for live subject human blood data (if it made the high threshold cut) than literature-derived human postmortem brain data, human genetic data, or the animal model data. The human blood-brain concordance is weighted more favorably than the animal model blood-brain concordance. The scoring analysis presented herein is just one example of assigning quantifiable values to prioritizing biomarkers for mood disorder analysis. Other ways of weighing the scores of line of evidence may give slightly different results in terms of prioritization, if not in terms of the list of genes per se.

The weightage given to a particular evidence, e.g., post-mortem data or blood expression may be varied. Additional scoring matrices may also be included to account for additional variables. One such example would be the temporal aspect—how long a particular biomarker is turned on.

### Example 4

### Clinical Applications

A sample, such as, 5-10 ml of blood is obtained from a patient suspected of having a mood disorder. RNA is isolated from the blood using standard protocols, for example with PAXgene blood RNA extraction kit (PreAnalytiX, a QIAGEN/BD company), followed by GLOBINclear™-Human or GLOBINclear™-Mouse/Rat (Ambion/Applied Biosystems Inc., Austin, Tex.) to remove the globin mRNA. Isolated RNA is labeled using any suitable detectable label if necessary for the gene expression analysis.

The labeled RNA is then quantified for the presence of one or more of the biomarkers disclosed herein. For example, gene expression analysis is performed using a panel of about 10 biomarkers (e.g., BioM 10 panel) by any standard technique, for example microarray analysis or quantitative PCR or an equivalent thereof. The gene expression levels are analyzed and the absent/present state or fold changes (either increased, decreased, or no change) are determined and a score is established

Applications of biomarkers for mood disorders: There are no reliable clinical laboratory blood tests for mood disorders. Given the complex nature of mood disorders, the current reliance on patient self-report of symptoms and the clinician's impression on interview of patient is a rate limiting step in delivering the best possible care with existing treatment modalities, as well as in developing new and improved treatment approaches, including new medications.

Biomarkers disclosed herein are used in the form of panels of biomarkers, as exemplified by a BioM-10 Mood panel, for clinical laboratory tests for mood disorders. Such tests can be: 1) at an mRNA level, quantitation of gene expression through polymerase chain reaction, 2) at a protein level, quantitation of protein levels through immunological approaches such as enzyme-linked immunosorbent assays (ELISA).

In conjunction with other clinical information, biomarker testing of blood and other fluids (CSF, urine) may play a desirable part of personalizing treatment to increase effectiveness and avoid adverse reactions—personalized medicine in psychiatry.

Biomarker-based tests for mood disorders help: 1) Diagnosis, early intervention and prevention efforts; 2) Prognosis and monitoring response to various treatments; 3) New neuropsychiatric drug development efforts by pharmaceutical companies, at both a pre-clinical and clinical (Phase I, II and III) stages of the process; 4) Identifying vulnerability to mood problems for people in high stress occupations (for example, military, police, homeland security).

### Example 4A

Diagnosis, early intervention and prevention efforts. A patient with no previous history of mood disorders presents to a primary care doctor or internist complaining of non-specific symptoms: low energy, fatigue, general malaise, aches and pains. Such symptoms are reported in conditions such as stress after a job loss, bereavement, mononucleosis, fibromyalgia, and postpartum in the general population, as well as Gulf War syndrome in veterans. A panel of mood biomarkers can substantiate that the patient is showing objective changes in the blood consistent with a low mood/depressive state. This will direct treatment towards, and substantiate the need to use, anti-depressant medications such as Prozac (fluoxetine), Zolof (sertraline), Celexa (citalopram), Cymbalta (duloxetine), Effexor (venlafaxine) or Wellbutrin (buproprion).

### Example 4B

Clinical diagnosis of a young patient. A young patient (child, adolescent, young adult) with no previous history of mood disorders, but coming from a family where one or more blood relatives suffer from depression may be monitored with regular laboratory tests by their primary care doctor/pediatrician using panels of mood biomarkers. These tests may detect early on a change towards decreased mood (depression) or towards increased mood (mania). This indicates and substantiates the need for initiation of medication treatment with anti-depressants (mentioned above), or mood stabilizers for mania—medication such as Depakote (divalproex), Lithobid (lithium), Lamictal (lamotrigene), Tegretol (carbamazepine), Topomax (topiramate). This early intervention may be helpful to prevent full-blown illness and hospitalizations, with their attendant negative medical and social consequences. The decision to start medications in children and adolescents is particularly difficult without objective proof, due to the potential side-effects of medications in that age group (agitation, suicidality, weigh-gain, sexual side-effects).

### Example 4C

Monitoring mood biomarkers over an extended period. Many patients with bipolar disorder may present initially with a depressive episode to their primary care doctor or psychiatrist. Monitoring mood biomarkers over time may also help to differentiate depression vs. bipolar disorder (manic-depression). This distinction is helpful because the first-line treatments for the two disorders are different: anti-depressants for depression, mood stabilizers for bipolar. If patients are miss-diagnosed as depressives instead of bipolars, and started on anti-depressant medications only, this can lead to activation and flip into manic states. If prior objective substantiation through biomarker testing of mood cyclicity (going up and down) existed, or early detection of mania in patients put on anti-depressants by seeing a change in biomarker profile towards a high mood state profile before full blown illness and clinical symptoms, an appropriate addition or change to a mood stabilizer medication can be implemented, preventing clinical decompensation, needles suffering and socio-economic loss (employment, relationships).

### Example 4D

Prognosis and monitoring response to various treatments. In depression, initiating medication treatment with current anti-depressants medications is a trial-and-error endeavor. It takes up to 6-8 weeks to see if a medication truly works. By doing a baseline biomarker panel test, and then a repeat test early one in treatment (after 1 week, for example), there would be an early objective indication if an anti-depressant is starting to work or not, and if a switch to another medication is indicated. This would save time and avoid needles suffering for patients, with the attendant socio-economic losses.

### Example 4E

Detecting loss of efficacy of an existing treatment. When a patient has been stable for a while on a medication for depression or bipolar disorder, regular biomarker testing may detect early loss of efficacy of the medication or recurrence of the illness, which would indicate the dose needs to be increased, medication changed, or another medication added, to prevent full blown clinical symptoms.

### Example 4F

Determining adequacy of treatment plan. Objective monitoring with blood biomarker panels of the effect of less reliable or evidence-based interventions: psychotherapy, lifestyle changes, diet and exercise programs for improving mood health. This will show whether the particular intervention works, is sufficient, or medications may need to be added to the regimen.

### Example 4G

Identifying vulnerability to developing mood problems for people in high stress occupations. Military personnel (recruits in boot-camp, active duty soldiers), other people in high-stress jobs (police, homeland security, astronauts), can be monitored on a regular basis to detect early objective changes in mood biomarker profile that would indicate the need for preventive intervention and/or the temporary removal from a high-stress environment.

### Example 5

### New Neuropsychiatric Drug Development

Early-stage pre-clinical work and clinical trials of new neuropsychiatric medications for treating mood disorders may benefit from biomarker monitoring to help make a decision early on whether the compound is working. This will speed up the drug-development process and avoid unnecessary costs. Depending on the expression profile of the biomarkers, the results of clinical trials may be obtained earlier than usual.

In later-stage large clinical trials, a new compound being tested may show an overall statistically non-significant positive effect, despite working well in a sub-group of people in the study. Biomarker testing may provide an objective signature of the genetic and biological make-up of the responders, which can inform recruitment for subsequent validatory clinical trials with higher likelihood of success, as well as inform which patients should be getting the medication, once it is FDA approved and on the market.

TABLE 1
Demographics: (a) individual (b) aggregate
Diagnosis established by DIGS comprehensive structured clinical interview.
(a) Individual demographic data.

| | | | | | |
|---|---|---|---|---|---|
| Subject ID | Diagnosis | Age | Gender | Ethnicity | VAS Mood (0-100) |

Primary Bipolar Cohort

| | | | | | |
|---|---|---|---|---|---|
| 174-1197-001 | BP | 37 | Male | Caucasian | 20 |
| 174-1055-001 | BP | 46 | Male | Caucasian | 20 |
| phchp029v1 | BP | 56 | Male | Caucasian | 22 |
| 174-1126-001 | BP | 33 | Male | Caucasian | 24 |
| 174-1173-001 | BP | 56 | Male | Caucasian | 27 |
| 174-1161-001 | BP | 46 | Male | Caucasian | 29 |
| 174-1150-001 | BP | 52 | Male | Caucasian | 31 |
| 174-1042-001 | BP | 58 | Male | Caucasian | 37 |
| 174-1112-001 | BP | 24 | Male | Caucasian | 38 |
| phchp027v1 | BP | 40 | Male | Caucasian | 38 |
| 174-1137-001 | BP | 48 | Male | African American | 39 |
| phchp023v1 | BP | 52 | Male | Caucasian | 39 |
| 174-1115-001 | BP | 42 | Male | American Indian | 40 |
| phchp020v1 | BP | 62 | Male | Caucasian | 42 |
| phchp031v1 | BP | 51 | Male | Caucasian | 47 |
| phchp028v1 | BP | 50 | Female | Asian | 52 |
| phchp030v1 | BP | 49 | Male | Caucasian | 61 |
| 174-1107-001 | BP | 39 | Male | Caucasian | 63 |
| 174-1130-001 | BP | 21 | Male | African American | 65 |
| 174-5001-001 | BP | 23 | Male | Caucasian | 66 |
| 174-1132-001 | BP | 22 | Male | African American | 71 |
| 174-1160-001 | BP | 52 | Male | Caucasian | 72 |
| 174-1171-001 | BP | 56 | Female | Caucasian | 72 |
| 174-1156-001 | BP | 57 | Male | Caucasian | 72 |
| 174-1037-001 | BP | 54 | Male | Caucasian | 72 |
| 174-5002-001 | BP | 26 | Male | Caucasian | 73 |
| 174-1119-001 | BP | 38 | Male | Caucasian | 73 |
| phchp020v2 | BP | 62 | Male | Caucasian | 80 |
| 174-1193-001 | BP | 53 | Male | African American | 84 |

Psychosis Cohort

| | | | | | |
|---|---|---|---|---|---|
| phchp022v2 | SZ | 48 | Male | Caucasian | 15 |
| phchp005v2 | SZA | 45 | Male | Caucasian | 19 |
| phchp025v1 | SZ | 42 | Male | Caucasian | 29 |
| phchp021v2 | SZA | 49 | Male | Hispanic | 29 |
| phchp006v2 | SZA | 52 | Male | African American | 33 |
| phchp033v1 | SZA | 48 | Male | Caucasian | 35 |
| phchp016v1 | SZ | 54 | Male | African American | 38 |
| phchp021v1 | SZA | 48 | Male | Hispanic | 39 |
| phchp019v1 | SubPD | 50 | Male | African-American | 41 |
| phchp003v3 | SZ | 50 | Male | African American | 47 |
| phchp010v1 | SZA | 45 | Male | Caucasian | 48 |
| phchp024v1 | SZA | 49 | Male | African American | 49 |
| phchp003v2 | SZ | 50 | Male | African American | 53 |
| phchp009v1 | SZ | 55 | Male | African American | 54 |
| phchp010v2 | SZA | 45 | Male | Caucasian | 55 |
| phchp006v1 | SZA | 52 | Male | African American | 57 |
| phchp026v1 | SZA | 49 | Male | African-American | 64 |
| phchp022v1 | SZ | 48 | Male | Caucasian | 65 |
| phchp010v3 | SZA | 45 | Male | Caucasian | 65 |
| phchp014v1 | SubPD | 55 | Male | African American | 69 |
| phchp004v1 | SZA | 55 | Male | African American | 69 |
| phchp012v1 | SZA | 55 | Male | Caucasian | 70 |
| phchp012v2 | SZA | 55 | Male | Caucasian | 71 |
| phchp018v1 | SZA | 54 | Female | Caucasian | 73 |
| phchp015v1 | SubPD | 48 | Male | African American | 76 |
| phchp008v1 | SZ | 47 | Male | African American | 76 |
| phchp005v1 | SZA | 45 | Male | Caucasian | 81 |
| phchp017v2 | SZA | 53 | Male | African American | 84 |
| phchp013v1 | SZA | 53 | Male | African American | 89 |
| phchp003v1 | SZ | 50 | Male | African American | 93 |

Secondary Bipolar Cohort

| | | | | | |
|---|---|---|---|---|---|
| phchp039v1 | BP | 52 | Male | Caucasian | 11 |
| phchp023v2 | BP | 52 | Male | Caucasian | 20 |
| 174-1216-001 | BP | 60 | Male | Caucasian | 23 |
| 174-1278-001 | BP | 22 | Male | Caucasian | 24 |
| 174-1232-001 | BP | 45 | Male | Caucasian | 32 |
| phchp045v1 | BP | 36 | Male | Caucasian | 36 |
| 174-1203-001 | BP | 39 | Male | African American | 49 |
| 174-1199-001 | BP | 41 | Male | Caucasian | 53 |
| 174-1237-001 | BP | 36 | Male | Caucasian | 57 |
| 174-5006-001 | BP | 60 | Male | Caucasian | 66 |
| phchp053v1 | BP | 58 | Male | Caucasian | 68 |
| 174-1211-001 | BP | 27 | Male | Caucasian | 75 |
| phchp031v2 | BP | 51 | Male | Caucasian | 79 |
| 174-1204-001 | BP | 52 | Male | Caucasian | 81 |
| 174-1255-001 | BP | 50 | Male | Caucasian | 81 |
| 174-1220-001 | BP | 68 | Male | Caucasian | 82 |
| 174-1096-001 | BP | 50 | Male | Caucasian | 83 |
| phchp056v1 | BP | 36 | Male | Caucasian | 84 |
| 174-1258-001 | BP | 36 | Male | Caucasian | 90 |

(b) Aggregate demographic data

| | | | | Psychosis Cohort | | | | | |
|---|---|---|---|---|---|---|---|---|---|
| | Primary Bipolar Cohort | | | | | Substance | | | |
| | BP | BP | | | | induced | Secondary Bipolar Cohort | | |
| | Low | High | BP | | | psychotic | BP | BP | BP |
| | Mood | Mood | Overall | SZA | SZ | disorder | Low Mood | High Mood | Overall |
| Number | 13 | 13 | 29 | 18 | 9 | 3 | 6 | 10 | 19 |
| of | | | | | | | | | |
| Subjects | | | | | | | | | |
| Gender | 13:0 | 12:1 | 27:2 | 17:1 | 9:0 | 3:0 | 6:0 | 10:0 | 19:0 |
| (males:females) | | | | | | | | | |
| Age | 45.4 | 41.9 | 45.0 | 49.8 | 49.3 | 51.0 | 44.5 | 48.8 | 45.8 |
| mean | (10.0) | (15.6) | (12.5) | (3.9) | (3.8) | (3.6) | (13.6) | (12.5) | (12.0) |
| years | 24 to 58 | 21 to 62 | 21 to 62 | 45 to 55 | 42 to 55 | 48 to 55 | 22 to 60 | 27 to 68 | 22 to 68 |
| (SD) range | | | | | | | | | |
| Duration | 22.8 | 20.4 | 21.6 | 31.2 | 26.7 | 25.0 | 25.8 | 27.2 | 25.8 |
| of | (10.2) | (17.1) | (13.7) | (6.3) | (4.7) | (6.0) | (16.1) | (6.6) | (10.5) |
| Illness | 5 to 40 | 2 to 49 | 2 to 49 | 17 to 42 | 20 to 26 | 20 to 32 | 7 to 53 | 19 to 38 | 7 to 53 |
| mean | | | | | | | | | |
| years | | | | | | | | | |
| (SD) range | | | | | | | | | |
| Ethnicity | 11/2 | 10/3 | 23/6 | 9/9 | 3/6 | 0/3 | 6/0 | 10/0 | 18/1 |
| (Caucasian/ | | | | | | | | | |
| Other) | | | | | | | | | |

BP—bipolar,
SubPD—substance induced psychosis,
SZ—schizophrenia,
SZA—schizoaffective disorder.
VAS Mood score at time of blood draw, on a scale 0 (lowest mood) to 100 (highest mood).

TABLE 2
High threshold and low threshold analysis in primary bipolar cohort and in meta-
analysis of both bipolar cohorts. Genes are considered candidate biomarkers for high mood if they
are called by the Affymetrix MAS5 software as Absent (A) in the blood of low mood subjects and
detected as Present (P) in the blood of high mood subjects. Conversely, genes are considered
candidate biomarkers for low mood if they are detected as Present (P) in low mood subjects and
Absent (A) in high mood subjects.

| | Bipolar Subjects (n = 29) |
|---|---|
| Primary Cohort Analysis | 13 Low Mood and 13 High Mood |
| High Threshold Candidate Biomarker Genes (changed in greater than or equal to | 10/13 Low Mood vs 10/13 High |
| 75% subjects; i.e. at least 3-fold enrichment) | Mood |
| | A/P and P/A analysis |
| Low Threshold Candidate Biomarker Genes (changed in greater than or equal to | 8/13 Low Mood vs 8/13 High |
| 60% subjects; i.e. at least 1.5-fold enrichment) | Mood |
| | A/P and P/A analysis |
| | Bipolar Subjects (n = 42) |
| Meta-analysis | 19 Low Mood and 23 High Mood |
| High Threshold Candidate Biomarker Genes (changed in greater than or equal to | 15/19 Low Mood vs 18/23 High |
| 75% subjects; i.e. at least 3-fold enrichment) | Mood |
| Low Threshold Candidate Biomarker Genes (changed in greater than or equal to | 12/19 Low Mood vs 14/23 High |
| 60% subjects; i.e. at least 1.5-fold enrichment) | Mood |

TABLE 3
Top candidate biomarker genes for mood prioritized by CFG score for multiple
independent lines of evidence.

| | | | | Hu. Br. and Bl. | | BP | BP | |
|---|---|---|---|---|---|---|---|---|
| | | Hu. | | Concordance/ | Hu. Genetic | Mouse | Mouse | |
| | Entrez | Bl | Hu. Postmortem | Co- | Linkage/ | Model | Model | CFG |
| Gene Symbol/Name | Gene ID | Data | Brain, LCL | Directionality | Association | Brain² | Blood | Score |
| Mbp | 4155 | I | Up (male) BP | Yes/Yes | 18q23 | | Cat-IV | 6 |
| myelin basic protein | | | Down (female) | | BP′ | | Meth | |
| | | | BP | | | | (I) | |
| | | | Down Bipolar | | | | | |
| Edg2 | 1902 | I | Down MDD | Yes/Yes | 9q31.3 | | | 5 |
| Endothelial | | | Down (PFC) BP | | BP | | | |
| differentiation, | | | Up (Parietal | | | | | |
| lysophosphatidic acid | | | Cortex) BP | | | | | |
| G-protein-coupled | | | | | | | | |
| receptor, 2 | | | | | | | | |
| Fgfr1 | 2260 | D | Up MDD | Yes/Yes | 8p12 | | | 5 |
| fibroblast growth | | | | | BP | | | |
| factor receptor 1 | | | | | | | | |
| Fzd3 | 7976 | I | Down BP | Yes/Yes | 8p21.1 | | | 5 |
| frizzled homolog 3 | | | | | BP | | | |
| (Drosophila) | | | | | | | | |
| Mag | 4099 | D | Down MDD | Yes/No | 19q13.12 | CP Cat- | | 5 |
| myelin-associated | | | | | Depression | IV Meth | | |
| glycoprotein | | | | | | (I) | | |
| Pmp22 | 5376 | D | Down MDD | Yes/No | 17p12 | CP Cat- | | 5 |
| peripheral myelin | | | | | BP | IV Meth | | |
| protein 22 | | | | | | (I) | | |
| Ugt8 | 7368 | D | Down MDD | Yes/No | 4q26 | CP Cat- | | 5 |
| UDP | | | | | BP | II (I) | | |
| glycosyltransferase 8 | | | | | | | | |
| (UDP-galactose | | | | | | | | |
| ceramide | | | | | | | | |
| galactosyltransferase) | | | | | | | | |
| Erbb3 | 2065 | D | Down MDD | Yes/No | 12q13.2 | | | 4 |
| Neuregulin receptor | ( | | Down BP | | Depression | | | |
| (v-erb-a erythroblastic | | | | | | | | |
| leukemia viral | | | | | | | | |
| oncogene homolog 4 | | | | | | | | |
| (avian)) | | | | | | | | |
| Igfbp4 | 3487 | D | Down BP | Yes/No | 17q21.2 | | | 4 |
| insulin-like growth | | | | | Depression | | | |
| factor binding protein 4 | | | | | | | | |
| Igfbp6 | 3489 | D | Down BP | Yes/No | 12q13 | | | 4 |
| insulin-like growth | | | | | Depression | | | |
| factor binding protein 6 | | | | | | | | |
| Pde6d | 5147 | D | Up BP | Yes/Yes | 2q37.1 | | | 4 |
| phosphodiesterase | | | | | | | | |
| 6D, cGMP-specific, | | | | | | | | |
| rod, delta | | | | | | | | |
| Ptprm | 5797 | D | Up BP | Yes/Yes | 18p11.23 | | | 4 |
| protein tyrosine | | | | | | | | |
| phosphatase, receptor | | | | | | | | |
| type, M | | | | | | | | |
| Nefh | 4744 | D | DownBP | Yes/No | 22q12.2 | | | 4 |
| neurofilament, heavy | | (MA) | | | | | | |
| polypeptide 200 kDa | | | | | | | | |
| Atp2c1 | 27032 | D | | | 3q21.3 | | | 3 |
| ATPase, Ca++- | | | | | BP | | | |
| sequestering | | | | | | | | |
| Atxn1 | 6310 | I | | | 6p22.3 | CP Cat- | | 3 |
| Ataxin 1 | | | | | BP | IV Meth | | |
| | | | | | | (D) | | |
| Btg1 | 694 | D | | | 12q21.33 | | Cat-III | 3 |
| B-cell translocation | | | | | BP | | Meth | |
| gene 1, anti- | | | | | | | (D) | |
| proliferative | | | | | | | | |
| C6orf182 | 285753 | D | | | 6q21 | | | 3 |
| chromosome 6 open | | | | | BP | | | |
| reading frame 182 | | | | | | | | |
| Dicer1 | 23405 | D | Down MDD | Yes/No | 14q32.13 | | | 3 |
| Dicer1, Dcr-1 | | | | | | | | |
| homolog (Drosophila) | | | | | | | | |
| Dnajc6 | 9829 | D | | | 1p31.3 | | | 3 |
| DnaJ (Hsp40) | | (HT) | | | BP | | | |
| homolog, subfamily C, | | | | | Depression | | | |
| member 6 | | | | | | | | |
| Ednrb | 1910 | I | | | 13q22.3 | CP Cat- | | 3 |
| endothelin receptor | | | | | BP | III Meth | | |
| type B | | | | | | (I) | | |
| Elovl5 | 60481 | D | | | 6p12.1 | | Cat-IV | 3 |
| ELOVL family | | | | | BP | | VPA | |
| member 5, elongation | | | | | | | (D) | |
| of long chain fatty | | | | | | | | |
| acids (yeast) | | | | | | | | |
| Gnal | 2774 | D | | | 18p11.21 | | | 3 |
| guanine nucleotide | | (HT) | | | BP | | | |
| binding protein, alpha | | | | | | | | |
| stimulating, olfactory | | | | | | | | |
| type | | | | | | | | |
| Klf5 | 688 | D | | | 13q22.1 | | | 3 |
| Kruppel-like factor 5 | | (HT) | | | BP | | | |
| Lin7a | 8825 | D | | | 12q21.31 | Increased | | 3 |
| lin 7 homolog a (C. elegans) | | | | | BP | (Rat | | |
| | | | | | | Brain) | | |
| Manea | 79694 | D | | | 6q16.1 | | | 3 |
| mannosidase, endo- | | (HT) | | | BP | | | |
| alpha | | | | | Depression | | | |
| Nupl1 | 9818 | D | | | 13q12.13 | | | 3 |
| nucleoporin like 1 | | (HT) | | | BP | | | |
| Pde6b | 5158 | D | Down MDD | Yes/No | 4p16.3 | | | 3 |
| phosphodiesterase | | | | | | | | |
| 6B, cGMP-specific, | | | | | | | | |
| rod, beta (congenital | | | | | | | | |
| stationary night | | | | | | | | |
| blindness 3, | | | | | | | | |
| autosomal dominant) | | | | | | | | |
| Slc25a23 | 79085 | D | | | 19p13.3 | CP Cat- | | 3 |
| solute carrier family | | (HT) | | | | IV VPA | | |
| 25 (mitochondrial | | | | | | (I) | | |
| carrier; phosphate | | | | | | | | |
| carrier), member 23 | | | | | | | | |
| Synpo | 11346 | D | | | 5q33.1 | PFC | | 3 |
| synaptopodin | | | | | BP | Cat-III | | |
| | | | | | | Meth | | |
| | | | | | | (D) | | |
| Tgm2 | 7052 | D | | | 20q11.23 | | Cat-III | 3 |
| transglutaminase 2, C | | | | | BP | | Meth | |
| polypeptide | | | | | | | (D) | |
| Tjp3 | 27134 | D | | | 19p13.3 | | | 3 |
| tight junction protein 3 | | (HT) | | | BP | | | |
| (zona occludens 3) | | | | | | | | |
| Tpd52 | 7163 | D | | | 8q21.13 | | | 3 |
| tumor protein D52 | | (HT) | | | BP | | | |
| Trpc1 | 7220 | D | | | 3q23 | CP Cat- | | 3 |
| transient receptor | | | | | BP | IV VPA | | |
| potential cation | | | | | | (I) | | |
| channel, subfamily C, | | | | | | | | |
| member 1 | | | | | | | | |
| Bclaf1 | 9774 | D | Down | | 6q23.3 | | | 2 |
| BCL2-associated | | | (Lymphocytes) | | | | | |
| transcription factor 1 | | | BP | | | | | |
| Gosr2 | 9570 | D | Down | | 17q21.32 | | | 2 |
| golgi SNAP receptor | | | (Lymphocytes) | | | | | |
| complex member 2 | | | BP | | | | | |
| Rdx | 5962 | D | Down | | 11q22.3 | | | 2 |
| radixin | | | (Lymphocytes) | | | | | |
| | | | BP | | | | | |
| Wdr34 | 89891 | D | Down | | 9q34.11 | | | 2 |
| WD repeat domain 34 | | | (Lymphocytes) | | | | | |
| | | | BP | | | | | |
| Bic | 114614 | D | | | 21q21.3 | | | 2 |
| BIC transcript | | (MA) | | | | | | |
| (microRNA 155) | | | | | | | | |
| C8orf42 | 157695 | D | | | 8p23.3 | | | 2 |
| chromosome 8 open | | (MA) | | | | | | |
| reading frame 42 | | | | | | | | |
| Dock9 | 23348 | D | | | 13q32.3 | | Cat-III- | 2 |
| Dedicator of | | (MA) | | | | | Val (D) | |
| cytokinesis 9 | | | | | | | | |
| Hrasls | 57110 | D | | | 3q29 | | | 2 |
| HRAS-like suppressor | | (MA) | | | | | | |
| Ibrdc2 | 255488 | D | | | 6p22.3 | | | 2 |
| IBR domain | | (MA) | | | | | | |
| containing 3 | | | | | | | | |
| (Rnf144b) | | | | | | | | |
| P2ry12 | 64805 | D | | | 3q25.1 | | | 2 |
| purinergic receptor | | (MA) | | | | | | |
| P2Y, G-protein | | | | | | | | |
| coupled 12 | | | | | | | | |
| Specc1 | 92521 | D | | | 17p11.2 | | | 2 |
| spectrin domain with | | (MA) | | | | | | |
| coiled-coils 1 | | | | | | | | |
| Vil2 | 7430 | I | | | 6q25.3 | | | 2 |
| villin 2 (ezrin) | | (MA) | | | | | | |
| C20orf7 | 79133 | D | | | 20p12.1 | | | 1 |
| chromosome 20 open | | (MA) | | | | | | |
| reading frame 7 | | | | | | | | |
| Chrnb3 | 1142 | I | | | 8p11.21 | | | 1 |
| cholinergic receptor, | | (MA) | | | | | | |
| nicotinic, beta 3 | | | | | | | | |
| Eif4a2 | 1974 | I | | | 3q27.3 | | | 1 |
| eukaryotic translation | | (MA) | | | | | | |
| initiation factor 4A, | | | | | | | | |
| isoform 2 | | | | | | | | |
| Gins4 | 84296 | I | | | 8p11.21 | | | 1 |
| GINS complex subunit | | (MA) | | | | | | |
| 4 (Sld5 homolog) | | | | | | | | |
| Grhl1 | 29841 | D | | | 2p25.1 | | | 1 |
| grainyhead-like 1 | | (MA) | | | | | | |
| (Drosophila) | | | | | | | | |
| Gtpbp8 | 29083 | D | | | 3q13.2 | | | 1 |
| GTP-binding protein 8 | | (MA) | | | | | | |
| (putative) | | | | | | | | |
| Heatr6 | 63897 | I | | | 17q23.2 | | | 1 |
| HEAT repeat | | (MA) | | | | | | |
| containing 6 | | | | | | | | |
| Igl@ | 3535 | I | | | 22q11.1-q11.2 | | | 1 |
| immunoglobulin | | (MA) | | | | | | |
| lambda chain, | | | | | | | | |
| variable 1 | | | | | | | | |
| II17rc | 84818 | I | | | 3p25.3 | | | 1 |
| interleukin 17 receptor C | | (MA) | | | | | | |
| Itfg1 | 81533 | D | | | 16q12.1 | | | 1 |
| integrin alpha 2b | | (MA) | | | | | | |
| Loc388692 | 388692 | D | | | 1q21.2 | | | 1 |
| hypothetical gene | | (MA) | | | | | | |
| supported by | | | | | | | | |
| AK123662 | | | | | | | | |
| Loc654342 | 654342 | I | | | 2p11.1 | | | 1 |
| Similar to lymphocyte- | | (MA) | | | | | | |
| specific protein 1 | | | | | | | | |
| Lrrc37a | 9884 | D | | | 17q21.31 | | | 1 |
| leucine rich repeat | | (MA) | | | | | | |
| containing 37A | | | | | | | | |
| Pbrm1 | 55193 | I | | | 3p21.1 | | | 1 |
| polybromo 1 | | (MA) | | | | | | |
| Pex13 | 5194 | D | | | 2p16.1 | | | 1 |
| peroxisome | | (MA) | | | | | | |
| biogenesis factor 13 | | | | | | | | |
| Pol3s | 339105 | I | | | 16p11.2 | | | 1 |
| polyserase 3 | | (MA) | | | | | | |
| Pparbp | 5469 | D | | | 17q12 | | | 1 |
| PPAR binding protein | | (MA) | | | | | | |
| Prkd2 | 25865 | D | | | 19q13.32 | | | 1 |
| protein kinase D2 | | (MA) | | | | | | |
| Prr7 | 80758 | D | | | 5q35.3 | | | 1 |
| proline rich 7 | | (MA) | | | | | | |
| (synaptic) | | | | | | | | |
| Psph | 5723 | D | | | 7p11.2 | | | 1 |
| phosphoserine | | (MA) | | | | | | |
| phosphatase | | | | | | | | |
| Rfx3 | 5991 | I | | | 9p24.2 | | | 1 |
| regulatory factor X, 3 | | (MA) | | | | | | |
| (influences HLA class | | | | | | | | |
| II expression) | | | | | | | | |
| Rps16 | 6217 | D | | | 19q13.2 | | | 1 |
| ribosomal protein S16 | | (MA) | | | | | | |
| Samd4a | 23034 | I | | | 14q22.2 | | | 1 |
| sterile alpha motif | | (MA) | | | | | | |
| domain containing 4A | | | | | | | | |
| Scamp1 | 9522 | D | | | 5q14.1 | | | 1 |
| secretory carrier | | (MA) | | | | | | |
| membrane protein 1 | | | | | | | | |
| Scn11a | 11280 | I | | | 3p22.2 | | | 1 |
| sodium channel, | | (MA) | | | | | | |
| voltage-gated, type | | | | | | | | |
| XI, alpha | | | | | | | | |
| Spa17 | 53340 | D | | | 11q24.2 | | | 1 |
| sperm autoantigenic | | (MA) | | | | | | |
| protein 17 | | | | | | | | |
| Tcf7l2 | 6934 | I | | | 10q25.3 | | | 1 |
| transcription factor 7- | | (MA) | | | | | | |
| like 2, T-cell specific, | | | | | | | | |
| HMG-box | | | | | | | | |
| Wbscr16 | 81554 | I | | | 7q11.23 | | | 1 |
| Williams-Beuren | | (MA) | | | | | | |
| syndrome | | | | | | | | |
| chromosome region | | | | | | | | |
| 16 | | | | | | | | |
| Wdr55 | 54853 | D | | | 5q31.3 | | | 1 |
| WD repeat domain 55 | | (MA) | | | | | | |
| Znf492 | 57615 | D | | | 19p12 | | | 1 |
| zinc finger protein 492 | | (MA) | | | | | | |
| Znf576 | 79177 | I | | | 19q13.31 | | | 1 |
| zinc finger protein 576 | | (MA) | | | | | | |

Top candidate biomarker genes for mood.
For human blood (Hu Bl.) data:
I—increased in high mood (mania);
D—decreased in high mood (mania)/increased in low mood (depression).
For human postmortem brain (Hu Br.) data:
Up—increased;
Down—decreased in expression.
For mouse data
METH—methamphetamine,
VPA—valproate.
MDD—major depressive disorder.
LCL—lymphoblastoid cell lines.
(HT) High threshold.
(MA) identified by meta-analysis only.

TABLE 4
BioM-10 Mood panel derived from primary bipolar cohort analysis:
sensitivity and specificity for predicting mood state. Primary Bipolar
cohort (A), Psychosis Cohort (B) and Secondary Bipolar cohort (C).
Results with meta-analysis derived panel (D).

| | |
|---|---|
| Sensitivity | Specificity |

A.

| | | |
|---|---|---|
| Primary Bipolar | | |
| Cohort | | |
| High Mood | 84.6% | 68.8% |
| Low Mood | 76.9% | 81.3% |

B.

| | | |
|---|---|---|
| Other Psychotic | | |
| Disorders Cohort | | |
| High Mood | 71.4% | 62.5% |
| Low Mood | 66.7% | 61.9% |

C.

| | | |
|---|---|---|
| Secondary Bipolar | | |
| Cohort | | |
| High Mood | 70% | 66.7% |
| Low Mood | 66.7% | 61.5% |

High Mood: Mbp, Edg2, Fzd3, Atxn1, Ednrb
Low Mood: Fgfr1, Mag, Pmp22, Ugt8, Erbb3

| | |
|---|---|
| Sensitivity | Specificity |

D.

| | | |
|---|---|---|
| Primary Bipolar Cohort | | |
| High Mood | 84.6% | 80.0% |
| Low Mood | 61.5% | 87.5% |
| Secondary Bipolar Cohort | | |
| High Mood | 90% | 88.9% |
| Low Mood | 66.7% | 92.3% |

Meta-analysis derived BioM 10 Mood Panel
High Mood: Edg2, Ednrb, Vil2, Bivm, Camk2d
Low Mood: Trpc1, Elovl5, Ugt8, Btg1, Nefh

TABLE 5
Biological Roles. Ingenuity pathway analysis (IPA) of biological functions
categories among our top blood candidate biomarker genes for mood.
Genes from Table 3. Top categories, over-
represented with a significance of p < 0.05, are shown.

| | |
|---|---|
| Cell Death | 1.43E−07-4.54E−02 |
| Nervous System Development and Function | 8.31E−07-4.63E−02 |
| Cell Morphology | 2.25E−05-4.63E−02 |
| Cellular Assembly and Organization | 4.48E−05-4.56E−02 |
| Neurological Disease | 7.46E−05-4.63E−02 |
| Cellular Growth and Proliferation | 1.11E−04-4.89E−02 |
| Skeletal and Muscular System | 1.11E−04-3.83E−02 |
| Development and Function | |
| Tissue Morphology | 1.12E−04-4.09E−02 |
| Behavior | 2.08E−04-4.63E−02 |
| Digestive System Development and Function | 2.08E−04-4.63E−02 |
| Cellular Development | 2.86E−04-4.63E−02 |
| Cancer | 5.50E−04-4.89E−02 |

TABLE 6
Targets of existing drugs. Complete list of the blood candidate biomarker genes
for mood that are the direct target of existing drugs.

| Genes | Gene Name | Drugs |
|---|---|---|
| ADA | adenosine deaminase | pentostatin |
| AGTR1 | angiotensin II receptor, type 1 | losartan/hydrochlorothiazide, valsartan/hydrochlorothiazide, |
| | | candesartan cilexetil, olmesartan medoxomil, irbesartan, |
| | | losartan potassium, telmisartan, eprosartan, candesartan |
| | | cilexetil/hydrochlorothiazide, hydrochlorothiazide/irbesartan, |
| | | eprosartan/hydro |
| COL6A2 | collagen, type VI, alpha 2 | collagenase |
| DHFR | dihydrofolate reductase | iclaprim, methotrexate, LY231514, PT 523 |
| EDNRB | endothelin receptor type B | bosentan, sitaxsentan, atrasentan |
| GNRH1 | gonadotropin-releasing hormone 1 | leuprolide, goserelin |
| | (luteinizing-releasing hormone) | |
| GNRHR | gonadotropin-releasing hormone | cetrorelix, triptorelin, abarelix |
| | receptor | |
| GUCY1A3 | guanylate cyclase 1, soluble, alpha 3 | nitroglycerin, isosorbide-5-mononitrate, isosorbide dinitrate, |
| | | nitroprusside, isosorbide dinitrate/hydralazine |
| KCNMB4 | potassium large conductance calcium- | tedisamil |
| | activated channel, subfamily M, beta | |
| | member 4 | |
| PDE4D | phosphodiesterase 4D, cAMP- | arofylline, tetomilast, anagrelide, cilomilast, milrinone, rolipram, L- |
| | specific (phosphodiesterase E3 dunce | 826,141, roflumilast, caffeine |
| | homolog, Drosophila) | |
| PDE5A | phosphodiesterase 5A, cGMP- | DA-8159, sildenafil, dipyridamole, aspirin/dipyridamole, |
| | specific | vardenafil, tadalafil |
| POLE | polymerase (DNA directed), epsilon | nelarabine, gemcitabine, clofarabine, trifluridine |
| PPARA | peroxisome proliferative activated | tesaglitazar, clofibrate, fenofibrate, gemfibrozil |
| | receptor, alpha | |
| SLC18A2 | solute carrier family 18 (vesicular | deserpidine/methyclothiazide, deserpidine, |
| | monoamine), member 2 | reserpine/trichlormethiazide, chlorothiazide/reserpine, |
| | | chlorthalidone/reserpine, |
| | | hydralazine/hydrochlorothiazide/reserpine, |
| | | hydroflumethiazide/reserpine, polythiazide/reserpine, |
| | | hydrochlorothiazide/reserpine, r |
| TLR9 | toll-like receptor 9 | PF-3512676 |

TABLE 7
Complete list of blood candidate biomarker genes for mood
derived from primary bipolar cohort analysis.

| | | Human Blood |
|---|---|---|
| Gene Symbol/Name | Entrez Gene ID | Data |
| Abca11 | 10348 | D |
| ATP-binding cassette, sub-family A (ABC1), member | | |
| 11 (pseudogene | | |
| Abhd6 | 57406 | D |
| abhydrolase domain containing 6 | | |
| Acacb | 32 | D |
| acetyl-Coenzyme A carboxylase beta | | |
| Adamts5 | 11096 | D |
| ADAM metallopeptidase with thrombospondin type 1 | | |
| motif, 5 (aggrecanase-2) | | |
| Agmat | 79814 | D |
| agmatine ureohydrolase (agmatinase) | | |
| Agpat7 | 254531 | D |
| 1-acylglycerol-3-phosphate O-acyltransferase 7 | | |
| (lysophosphatidic acid acyltransferase, eta) | | |
| Agrn | 375790 | D |
| agrin | | |
| Agtr1 | 185 | D |
| angiotensin II receptor, type 1 | | |
| Amn | 81693 | D (HT) |
| Amnionless homolog (mouse) | | |
| Anapc10 | 10393 | D |
| anaphase promoting complex subunit 10 | | |
| Ankdd1a | 348094 | I |
| ankyrin repeat and death domain containing 1A | | |
| Ankrd13b | 124930 | D |
| ankyrin repeat domain 13B | | |
| Ankrd22 | 118932 | I |
| ankyrin repeat domain 22 | | |
| Ankrd54 | 129138 | D |
| ankyrin repeat domain 54 | | |
| Ankrd57 | 65124 | D |
| ankyrin repeat domain 57 | | |
| Anubl1 | 93550 | D |
| AN1, ubiquitin-like, homolog (Xenopus laevis) | | |
| Apobec4 | 403314 | I |
| apolipoprotein B mRNA editing enzyme, catalytic | | |
| polypeptide-like 4 (putative) | | |
| Arid1b | 57492 | D |
| AT rich interactive domain 1B (SWI1-like) | | |
| Armc8 | 25852 | D |
| armadillo repeat containing 8 | | |
| Arsk | 153642 | D |
| arylsulfatase family, member K | | |
| Atad2 | 29028 | D |
| ATPase family, AAA domain containing 2 | | |
| Atp2c1 | 27032 | D |
| ATPase, Ca++-sequestering | | |
| Atp6v1e2 | 90423 | D |
| ATPase, H+ transporting, lysosomal 31 kDa, V1 | | |
| subunit E2 | | |
| Atp7b | 540 | D |
| ATPase, Cu++ transporting, beta polypeptide | | |
| Atxn 1 | 6310 | I |
| Ataxin 1 | | |
| Azi2 | 64343 | D |
| 5-azacytidine induced gene 2 | | |
| B3gnt1 | 11041 | D |
| UDP-GlcNAc:betaGal beta-1,3-N- | | |
| acetylglucosaminyltransferase 1 | | |
| Bcas4 | 55653 | D |
| breast carcinoma amplified sequence 4 | | |
| Bclaf1 | 9774 | D |
| BCL2-associated transcription factor 1 | | |
| Bet3l | 221300 | D |
| BET3 like (S. cerevisiae | | |
| Bhlhb3 | 79365 | D |
| basic helix-loop-helix domain containing, class B, 3 | | |
| Bic | 114614 | D |
| BIC transcript | | |
| Bivm | 54841 | I |
| basic, immunoglobulin-like variable motif containing | | |
| Bmpr1a | 657 | D |
| bone morphogenetic protein receptor, type 1A | | |
| Bnip1 | 662 | D |
| BCL2/adenovirus E1B 19 kDa interacting protein 1 | | |
| Brwd 1 | 54014 | D |
| bromodomain and WD repeat domain containing 1 | | |
| Btbd12 | 84464 | I |
| BTB (POZ) domain containing 12 | | |
| Btg1 | 694 | D |
| B cell translocation gene 1, anti-proliferative | | |
| Btnl9 | 153579 | D |
| butyrophilin-like 9 | | |
| C10orf110 | 55853 | I |
| chromosome 10 open reading frame 110 | | |
| C10orf18 | 54906 | I |
| chromosome 10 open reading frame 18 | | |
| C11orf71 | 54494 | D |
| chromosome 11 open reading frame 71 | | |
| C11orf74 | 119710 | D |
| chromosome 11 open reading frame 74 | | |
| C12orf29 | 91298 | D |
| chromosome 12 open reading frame 29 | | |
| C12orf47 | 51275 | D |
| chromosome 12 open reading frame 47 | | |
| C14orf118 | 55668 | D |
| chromosome 14 open reading frame 118 | | |
| C14orf131 | 55778 | D |
| chromosome 14 open reading frame 131 | | |
| C14orf145 | 145508 | D |
| chromosome 14 open reading frame 145 | | |
| C14orf64 | 388011 | D |
| chromosome 14 open reading frame 64 | | |
| C16orf52 | 146174 | D |
| chromosome 16 open reading frame 52 | | |
| C18orf1 | 753 | D |
| Chromosome 18 open reading frame 1 | | |
| C18orf25 | 147339 | I |
| chromosome 18 open reading frame 25 | | |
| C18orf55 | 29090 | D |
| chromosome 18 open reading frame 55 | | |
| C19orf52 | 90580 | I |
| chromosome 19 open reading frame 52 | | |
| C1orf89 | 79363 | D |
| chromosome 1 open reading frame 89 | | |
| C20orf112 | 140688 | D |
| chromosome 20 open reading frame 112 | | |
| C20orf94 | 128710 | I |
| chromosome 20 open reading frame 94 | | |
| C21orf109 | 193629 | D |
| chromosome 21 open reading frame 109 /// similar to | | |
| Protein C21orf109 | | |
| C21orf114 | 193629 | D |
| chromosome 21 open reading frame 114 | | |
| C21orf56 | 84221 | I |
| chromosome 21 open reading frame 56 | | |
| C2orf40 | 84417 | D |
| chromosome 2 open reading frame 40 | | |
| C3orf23 | 285343 | D |
| chromosome 3 open reading frame 23 | | |
| C6orf170 | 221322 | D |
| chromosome 6 open reading frame 170 | | |
| C6orf182 | 285753 | D |
| chromosome 6 open reading frame 182 | | |
| C6orf26 | 401251 | D |
| chromosome 6 open reading frame 26 | | |
| C6orf60 | 79632 | D |
| chromosome 6 open reading frame 60 | | |
| C7orf26 | 79034 | D |
| chromosome 7 open reading frame 26 | | |
| C7orf36 | 57002 | D |
| chromosome 7 open reading frame 36 | | |
| C8orf33 | 65265 | D |
| chromosome 8 open reading frame 33 | | |
| C9orf61 | 9413 | I |
| chromosome 9 open reading frame 61 | | |
| C9orf71 | 169693 | D |
| chromosome 9 open reading frame 71 | | |
| C9orf82 | 79886 | D |
| chromosome 9 open reading frame 82 | | |
| C9orf90 | 203245 | I |
| chromosome 9 open reading frame 90 | | |
| Cadm1 | 23705 | D |
| cell adhesion molecule 1 | | |
| Camk2d | 817 | I |
| Calcium/calmodulin-dependent protein kinase (CaM | | |
| kinase) II delta | | |
| Catsper2 | 117155 | D |
| cation channel, sperm associated 2 | | |
| Cbfb | 865 | D |
| core binding factor beta | | |
| Cc2d2a/Kiaa1345 | 57545 | D |
| KIAA1345 protein | | |
| Ccdc6 | 8030 | D |
| coiled-coil domain containing 6 | | |
| Ccdc65 | 85478 | D |
| coiled-coil domain containing 65 | | |
| Ccdc88a | 55704 | D |
| coiled-coil domain containing 88A | | |
| Ccdc99 | 54908 | D |
| coiled-coil domain containing 99 | | |
| Ccne2 | 9134 | D |
| cyclin E2 | | |
| Cdc7 | 8317 | D |
| cell division cycle 7 (S. cerevisiae) | | |
| Cdkn2b | 1030 | D |
| cyclin-dependent kinase inhibitor 2B (p15, inhibits | | |
| CDK4) | | |
| Ceacam6 | 4680 | D |
| carcinoembryonic antigen-related cell adhesion | | |
| molecule 6 (non-specific cross reacting antigen) | | |
| Cfl2 | 1073 | D |
| cofilin 2 (muscle) | | |
| Clcf1 | 23529 | D |
| cardiotrophin-like cytokine factor 1 | | |
| Clcn3 | 1182 | D |
| chloride channel 3 | | |
| Cllu1 | 574028 | D |
| chronic lymphocytic leukemia up-regulated 1 | | |
| Cmtm4 | 146223 | D |
| CKLF-like MARVEL transmembrane domain | | |
| containing 4 | | |
| Cnnm1 | 26507 | D |
| cyclin M1 | | |
| Cnot2 | 4848 | D |
| CCR4-NOT transcription complex, subunit 2 | | |
| Col6a2 | 1292 | D |
| procollagen, type VI, alpha 2 | | |
| Coq3 | 51805 | D |
| coenzyme Q3 homolog, methyltransferase (S. cerevisiae) | | |
| Cplx3 | 594855 | I |
| complexin 3 | | |
| Cpm | 1368 | I |
| carboxypeptidase M | | |
| Cpvl | 54504 | D |
| Carboxypeptidase, vitellogenic-like | | |
| Cr2 | 1380 | D |
| complement component (3d/Epstein Barr virus) | | |
| receptor 2 | | |
| Csnk1a1 | 1452 | D |
| casein kinase 1, alpha 1 | | |
| Ctr9 | 9646 | D |
| Ctr9, Paf1/RNA polymerase II complex component, | | |
| homolog (S. cerevisiae) | | |
| Cuedc1 | 404093 | I |
| CUE domain containing 1 | | |
| Cwf19l2 | 143884 | I |
| CWF19-like 2, cell cycle control (S. pombe) | | |
| Cxcl12 | 6387 | D |
| chemokine (C—X—C motif) ligand 12 | | |
| Cxcl6 | 6372 | D |
| chemokine (C—X—C motif) ligand 6 (granulocyte | | |
| chemotactic protein 2) | | |
| Cyp2c19 | 1557 | I |
| CYP2C9 cytochrome P450, family 2, subfamily C, | | |
| polypeptide 19 | | |
| Cyp2c9 | 1559 | D |
| cytochrome P450, family 2, subfamily C, polypeptide 9 | | |
| Cyp2e1 | 1571 | D |
| cytochrome P450, family 2, subfamily E, polypeptide 1 | | |
| Cyp2u1 | 113612 | D |
| cytochrome P450, family 2, subfamily U, polypeptide 1 | | |
| Daam1 | 23002 | D |
| dishevelled associated activator of morphogenesis 1 | | |
| Dcbld1 | 285761 | D |
| discoidin, CUB and LCCL domain containing 1 | | |
| Depdc6 | 64798 | D |
| DEP domain containing 6 | | |
| Dhfr | 1719 | D |
| dihydrofolate reductase | | |
| Dhx35 | 60625 | D |
| DEAH (Asp-Glu-Ala-His) box polypeptide 35 | | |
| Dicer1 | 23405 | D |
| Dicer1, Dcr-1 homolog (Drosophila) | | |
| Dio2 | 1734 | I |
| deiodinase, iodothyronine, type II | | |
| Dip2b | 57609 | I |
| DIP2 disco-interacting protein 2 homolog B | | |
| (Drosophila) | | |
| Disp1 | 84976 | D |
| dispatched homolog 1 (Drosophila) | | |
| DKFZp564H213 | 440432 | I |
| hypothetical gene supported by AL049275 | | |
| Dnajb9 | 4189 | D |
| DnaJ (Hsp40) homolog, subfamily B, member 9 | | |
| Dnajc6 | 9829 | D (HT) |
| DnaJ (Hsp40) homolog, subfamily C, member 6 | | |
| Dock5 | 80005 | D |
| dedicator of cytokinesis 5 | | |
| Dscaml1 | 57453 | D |
| Down syndrome cell adhesion molecule-like 1 | | |
| Dst | 667 | D |
| dystonin | | |
| Dtwd1 | 56986 | D |
| DTW domain containing 1 | | |
| Dtx3 | 196403 | D |
| deltex 3 homolog (Drosophila) | | |
| Dus4l | 11062 | D |
| dihydrouridine synthase 4-like (S. cerevisiae) | | |
| Dynlrb1 | 83658 | D |
| dynein, light chain, roadblock-type 1 | | |
| E2f5 | 1875 | D |
| E2F transcription factor 5, p130-binding | | |
| E2f7 | 144455 | D |
| E2F transcription factor 7 | | |
| Edg2 | 1902 | I |
| endothelial differentiation, lysophosphatidic acid G- | | |
| protein-coupled receptor, 2 | | |
| Ednrb | 1910 | I |
| endothelin receptor type B | | |
| Egr1 | 1958 | D |
| early growth response 1 | | |
| Eid3 | 493861 | D |
| E1A-like inhibitor of differentiation 3 | | |
| Eif4g3 | 8672 | D |
| eukaryotic translation initiation factor 4 gamma, 3 | | |
| Elovl5 | 60481 | D |
| ELOVL family member 5, elongation of long chain fatty | | |
| acids (yeast) | | |
| Emid1 | 129080 | D |
| EMI domain containing 1 | | |
| Emilin2 | 84034 | D |
| elastin microfibril interfacer 2 | | |
| Eml5 | 161436 | D |
| echinoderm microtubule associated protein like 5 | | |
| Enpp4 | 22875 | D |
| ectonucleotide pyrophosphatase/phosphodiesterase 4 | | |
| (putative function) | | |
| Entpd4 | 9583 | D |
| ectonucleoside triphosphate diphosphohydrolase 4 | | |
| Epb41l4a | 64097 | D |
| erythrocyte membrane protein band 4.1 like 4A | | |
| Epn1 | 29924 | D |
| epsin 1 | | |
| Eps8 | 2059 | D |
| epidermal growth factor receptor pathway substrate 8 | | |
| Erbb3 | 2065 | D |
| Neuregulin receptor (v-erb-a erythroblastic leukemia | | |
| viral oncogene homolog 4 (avian) | | |
| Espn | 83715 | D |
| espin | | |
| Ezh1 | 2145 | I |
| enhancer of zeste homolog 1 (Drosophila) | | |
| Fa2h | 79152 | I |
| fatty acid 2-hydroxylase | | |
| Faah2 | 158584 | D |
| fatty acid amide hydrolase 2 | | |
| Fam13a1 | 10144 | D |
| family with sequence similarity 13, member A1 | | |
| Fam55a | 120400 | I |
| family with sequence similarity 55, member A | | |
| Fam63b | 54629 | D |
| family with sequence similarity 63, member B | | |
| Fam98a | 25940 | D |
| family with sequence similarity 98, member A | | |
| Fam108c1 | 58489 | D |
| family with sequence similarity 108, member C1 | | |
| Fam120a | 23196 | D |
| family with sequence similarity 120A | | |
| Fam139A/Flj40722 | 285966 | D |
| hypothetical protein FLJ40722 | | |
| Fastk | 10922 | D |
| Fas-activated serine/threonine kinase | | |
| Fbxo15 | 201456 | D |
| F-box protein 15 | | |
| Fbxo31 | 79791 | D |
| F-box protein 31 | | |
| Fbxo5 | 26271 | D |
| F-box protein 5 | | |
| Fcer2 | 2208 | D |
| Fc fragment of IgE, low affinity II, receptor for (CD23) | | |
| Fchsd1 | 89848 | D |
| FCH and double SH3 domains 1 | | |
| Fcrl2 | 79368 | D |
| Fc receptor-like 2 | | |
| Fer1l3 | 26509 | D |
| fer-1-like 3, myoferlin (C. elegans) | | |
| Fgfr1 | 2260 | D |
| fibroblast growth factor receptor 1 | | |
| Fggy/Flj10986 | 55277 | I |
| hypothetical protein FLJ10986 | | |
| Flj13305 | 84140 | D |
| hypothetical protein FLJ13305 | | |
| Flj22167 | 79583 | D |
| hypothetical protein FLJ22167 | | |
| Flnc | 2318 | D |
| filamin C, gamma (actin binding protein 280 | | |
| Fndc3b | 64778 | I |
| fibronectin type III domain containing 3B | | |
| Fosl2 | 2355 | D |
| FOS-like antigen 2 | | |
| Fsd1l | 83856 | D |
| Fibronectin type III and SPRY domain containing 1- | | |
| like | | |
| Fzd3 | 7976 | I |
| frizzled homolog 3 (Drosophila) | | |
| G3bp1 | 10146 | D |
| GTPase activating protein (SH3 domain) binding | | |
| protein 1 | | |
| Gata3 | 2625 | D |
| GATA binding protein 3 | | |
| Gins3 | 64785 | D |
| GINS complex subunit 3 (Psf3 homolog) | | |
| Gm2a | 2760 | D |
| GM2 ganglioside activator | | |
| Gmppb | 29925 | D |
| GDP-mannose pyrophosphorylase B | | |
| Gnal | 2774 | D (HT) |
| guanine nucleotide binding protein, alpha stimulating, | | |
| olfactory type | | |
| Gng8 | 94235 | D |
| guanine nucleotide binding protein (G protein), gamma | | |
| 8 subunit | | |
| Gnrh1 | 2796 | D |
| gonadotropin-releasing hormone 1 (luteinizing- | | |
| releasing hormone) | | |
| Gnrhr | 2798 | D |
| gonadotropin-releasing hormone receptor | | |
| Golga2l1 | 55592 | D |
| golgi autoantigen, golgin subfamily a, 2-like 1 | | |
| Golga3 | 2802 | D |
| golgi autoantigen, golgin subfamily a, 3 | | |
| Golga8g | 283768 | D |
| golgi autoantigen, golgin subfamily a, 8G | | |
| Gosr2 | 9570 | D |
| golgi SNAP receptor complex member 2 | | |
| Gp1bb | 2812 | I |
| glycoprotein Ib (platelet), beta polypeptide | | |
| Gpatch2 | 55105 | D |
| G patch domain containing 2 | | |
| Gpd2 | 2820 | D |
| glycerol phosphate dehydrogenase 2, mitochondrial | | |
| Gpr180 | 160897 | D |
| G protein-coupled receptor 180 | | |
| Gpr19 | 2842 | D |
| G protein-coupled receptor 19 | | |
| Gpsm1 | 26086 | D |
| G-protein signalling modulator 1 (AGS3-like, C. elegans) | | |
| Gramd2 | 196996 | D |
| GRAM domain containing 2 | | |
| Grb2 | 2885 | D |
| growth factor receptor-bound protein 2 | | |
| Gtdc1 | 79712 | I |
| glycosyltransferase-like domain containing 1 | | |
| Habp4 | 22927 | D |
| hyaluronan binding protein 4 | | |
| Hells | 3070 | D |
| helicase, lymphoid-specific | | |
| Hemk1 | 51409 | D |
| HemK methyltransferase family member 1 | | |
| Herpud2 | 64224 | D |
| HERPUD family member 2 | | |
| Hif1an | 55662 | D |
| hypoxia-inducible factor 1, alpha subunit inhibitor | | |
| Hist1h3b | 8358 | D |
| histone cluster 1, H3b | | |
| Hla-dqa1 | 3117 | I |
| major histocompatibility complex, class II, DQ alpha 1 | | |
| /// major histocompatibility complex, class II, DQ alpha 1 | | |
| Hla-drb1 | 3123 | I |
| major histocompatibility complex, class II, DR beta 1 | | |
| Hps3 | 84343 | D |
| Hermansky-Pudlak syndrome 3 | | |
| Hrasls3 | 11145 | D |
| HRAS-like suppressor 3 | | |
| Huwe1 | 10075 | D |
| HECT, UBA and WWE domain containing 1 | | |
| Ica1 | 3382 | D |
| intestinal cell kinase | | |
| Ift172 | 26160 | D |
| intraflagellar transport 80 homolog (Chlamydomonas) | | |
| Igfbp4 | 3487 | D |
| insulin-like growth factor binding protein 6 | | |
| Igfbp6 | 3489 | D |
| Immunoglobulin heavy chain 1a (serum IgG2a) | | |
| Ighg1 | 3500 | D |
| immunoglobulin heavy constant gamma 1 (G1m | | |
| marker) | | |
| Igsf22 | 283284 | D |
| immunoglobulin superfamily, member 3 | | |
| Il15 | 3600 | D |
| interleukin 17 receptor A | | |
| Immp1l | 196294 | D |
| inner membrane protein, mitochondrial | | |
| Insc | 387755 | D |
| insulin induced gene 1 | | |
| Insr | 3643 | D |
| insulin receptor | | |
| Ints10 | 55174 | D |
| integrator complex subunit 7 | | |
| Ints7 | 25896 | D |
| integrator complex subunit 8 | | |
| Ipo11 | 51194 | D |
| Intracisternal A particle-promoted polypeptide | | |
| Itch | 83737 | D |
| Integrin alpha FG-GAP repeat containing 1 | | |
| Itsn2 | 50618 | I |
| isovaleryl Coenzyme A dehydrogenase | | |
| Jag1 | 182 | D |
| jagged 2 | | |
| Jakmip2 | 9832 | D |
| jumonji, AT rich interactive domain 1B | | |
| Jmjd5 | 79831 | D |
| junction-mediating and regulatory protein | | |
| Josd2 | 126119 | D |
| Josephin domain containing 2 | | |
| Katnal1 | 84056 | D |
| katanin p60 subunit A-like 1 | | |
| Kbtbd3 | 143879 | D |
| kelch repeat and BTB (POZ) domain containing 3 | | |
| Kcnmb4 | 27345 | D |
| potassium large conductance calcium-activated | | |
| channel, subfamily M, beta member 4 | | |
| Khk | 3795 | D |
| ketohexokinase (fructokinase) /// ketohexokinase | | |
| (fructokinase) | | |
| Kiaa0494 | 9813 | D |
| KIAA0494 | | |
| Kiaa1009 | 22832 | D |
| KIAA1009 | | |
| Kiaa1107 | 23285 | D |
| KIAA1107 | | |
| Kiaa1377 | 57562 | D |
| KIAA1377 | | |
| Kiaa1586 | 57691 | D |
| KIAA1586 | | |
| Kiaa1704 | 55425 | D |
| 1200011I18Rik KIAA1704 | | |
| Kiaa1729 | 85460 | D |
| KIAA1729 protein | | |
| Kif5c | 3800 | D |
| kinesin family member 5C | | |
| Klf12 | 11278 | D |
| Kruppel-like factor 12 | | |
| Klf5 | 688 | D (HT) |
| Kruppel-like factor 5 | | |
| Klk7 | 5650 | D |
| kallikrein-related peptidase 7 | | |
| Krtap4-9 | 85286 | I |
| keratin associated protein 4-9 | | |
| L2hgdh | 79944 | D |
| L-2-hydroxyglutarate dehydrogenase | | |
| Laptm4b | 55353 | D |
| lysosomal associated protein transmembrane 4 beta | | |
| Larp4 | 113251 | D |
| La ribonucleoprotein domain family, member 4 | | |
| Lepr | 3953 | I |
| leptin receptor | | |
| Lgals4 | 3960 | D |
| lectin, galactoside-binding, soluble, 4 (galectin 4) | | |
| Lhx4 | 89884 | I |
| LIM homeobox 4 | | |
| Lims2 | 55679 | D |
| LIM and senescent cell antigen-like domains 2 | | |
| Lin7a | 8825 | D |
| lin 7 homolog a (C. elegans) | | |
| Lin7b | 64130 | D |
| lin-7 homolog B (C. elegans) | | |
| Lins1 | 55180 | D |
| lines homolog 1 (Drosophila) | | |
| Loc144481 | 144481 | D |
| hypothetical protein LOC144481 | | |
| Loc144874 | 144874 | D |
| Hypothetical protein LOC144874 | | |
| Loc145783 | 145783 | D |
| hypothetical protein LOC145783 | | |
| Loc148709 | 148709 | D |
| actin pseudogene | | |
| Loc158863 | 158863 | D |
| hypothetical protein LOC158863 | | |
| Loc253012 | 253012 | D |
| hypothetical protein LOC253012 | | |
| Loc253039 | 253039 | D |
| hypothetical protein LOC253039 | | |
| Loc283140 | 283140 | I |
| hypothetical protein LOC283140 | | |
| Loc283481 | 283481 | D |
| hypothetical protein LOC283481 | | |
| Loc284373 | 284373 | D |
| hypothetical protein LOC284373 | | |
| Loc284749 | 284749 | D |
| hypothetical protein LOC284749 | | |
| Loc285014 | 285014 | D |
| hypothetical protein LOC285014 | | |
| Loc285378 | 285378 | D |
| hypothetical protein LOC285378 | | |
| Loc285535 | 285535 | D |
| hypothetical protein LOC285535 | | |
| Loc285813 | 285813 | D |
| hypothetical protein LOC285813 | | |
| Loc285831 | 285831 | D |
| hypothetical protein LOC285831 | | |
| Loc338653 | 338653 | I |
| hypothetical protein LOC338653 | | |
| Loc339803 | 339803 | D |
| hypothetical protein LOC339803 | | |
| Loc340544 | 340544 | D |
| hypothetical protein LOC340544 | | |
| Loc344405 | 344405 | D |
| hypothetical LOC344405 | | |
| Loc348180 | 348180 | D |
| hypothetical protein LOC348180, isoform 1 | | |
| Loc387647 | 387647 | D |
| hypothetical gene supported by BC014163 | | |
| Loc388692 | 388692 | D |
| hypothetical gene supported by AK123662 | | |
| Loc401913 | 401913 | I |
| hypothetical LOC401913 | | |
| Loc441383 | 441383 | D |
| hypothetical gene supported by AF086559; BC065734 | | |
| Loc442257 | 442257 | D |
| similar to 40S ribosomal protein S4, Y isoform 2 | | |
| Loc51035 | 51035 | D |
| SAPK substrate protein 1 | | |
| Loc51255 | 51255 | D |
| hypothetical protein LOC51255 | | |
| Loc554203 | 554203 | I |
| hypothetical LOC554203 | | |
| Loc554206 | 554206 | D |
| hypothetical LOC554206 | | |
| Loc56755 | 56755 | D |
| hypothetical protein LOC56755 | | |
| Loc619208 | 619208 | D |
| hypothetical protein LOC619208 | | |
| Loc645513 | 645513 | D |
| Similar to septin 7 | | |
| Loc730202 | 730202 | D |
| hypothetical protein LOC730202 | | |
| Loc91431 | 91431 | I |
| prematurely terminated mRNA decay factor-like | | |
| Loh3cr2a | 29931 | I |
| loss of heterozygosity, 3, chromosomal region 2, gene A | | |
| Lrp16 | 28992 | D |
| LRP16 protein | | |
| Lrrc16 | 55604 | D |
| leucine rich repeat containing 16 | | |
| Lrrc8a | 56262 | I |
| leucine rich repeat containing 8 family, member A | | |
| Lrrc8b | 23507 | D (HT) |
| leucine rich repeat containing 8 family, member B | | |
| Lrrcc1 | 85444 | D |
| leucine rich repeat and coiled-coil domain containing 1 | | |
| Lrrk1 | 79705 | I |
| leucine-rich repeat kinase 1 | | |
| Luzp1 | 7798 | D |
| leucine zipper protein 1 | | |
| Lyrm4 | 57128 | D |
| LYR motif containing 4 | | |
| Maf | 4094 | D |
| avian musculoaponeurotic fibrosarcoma (v-maf) AS42 | | |
| oncogene homolog | | |
| Mag | 4099 | D |
| myelin-associated glycoprotein | | |
| Manea | 79694 | D (HT) |
| mannosidase, endo-alpha | | |
| Mbd5 | 55777 | D |
| methyl-CpG binding domain protein 5 | | |
| Mbp | 4155 | I |
| myelin basic protein | | |
| Mcf2l | 23263 | D |
| MCF.2 cell line derived transforming sequence-like | | |
| Mcm3ap | 8888 | I |
| minichromosome maintenance complex component 3 | | |
| associated protein | | |
| Mcoln3 | 55283 | D |
| mucolipin 3 | | |
| Mds2 | 259283 | D |
| myelodysplastic syndrome 2 translocation associated | | |
| Me3 | 10873 | D |
| malic enzyme 3, NADP(+)-dependent, mitochondrial | | |
| Mfrp | 83552 | D |
| membrane frizzled-related protein | | |
| Mgat4a | | |
| mannosyl (alpha-1,3-)-glycoprotein beta-1,4-N- | 11320 | D |
| acetylglucosaminyltransferase, isozyme A | | |
| Mgc10997 | 84741 | D |
| MGC10997 | | |
| Mgc33556 | 339541 | I |
| hypothetical LOC339541 | | |
| Mgc39900 | 286527 | D |
| hypothetical protein MGC39900 | | |
| Mgc46336 | 283933 | D |
| hypothetical protein MGC46336 | | |
| Mia | 8190 | D |
| melanoma inhibitory activity | | |
| Mical3 | 57553 | D |
| microtubule associated monoxygenase, calponin and | | |
| LIM domain containing 3 | | |
| Mier3 | 166968 | D |
| mesoderm induction early response 1, family member 3 | | |
| Mki67 | 4288 | D |
| antigen identified by monoclonal antibody Ki-67 | | |
| Mks1 | 54903 | D |
| Meckel syndrome, type 1 | | |
| Mllt4 | 4301 | I |
| myeloid/lymphoid or mixed lineage-leukemia | | |
| translocation to 4 homolog (Drosophila) | | |
| Mmaa | 166785 | D |
| methylmalonic aciduria (cobalamin deficiency) cblA | | |
| type | | |
| Mmd2 | 221938 | I |
| monocyte to macrophage differentiation-associated 2 | | |
| Mocs2 | 4338 | D |
| molybdenum cofactor synthesis 2 | | |
| Morn3 | 283385 | D |
| MORN repeat containing 3 | | |
| Mrpl30 | 51263 | D |
| mitochondrial ribosomal protein L30 | | |
| Mrps15 | 64960 | I |
| mitochondrial ribosomal protein S15 | | |
| Mta3 | 57504 | D |
| metastasis associated 1 family, member 3 | | |
| Mtap | 4507 | D |
| methylthioadenosine phosphorylase | | |
| Mtfr1 | 9650 | D |
| mitochondrial fission regulator 1 | | |
| Mtmr3 | 8897 | D |
| myotubularin related protein 3 | | |
| Mustn1 | 389125 | D |
| musculoskeletal, embryonic nuclear protein 1 | | |
| Mxra8 | 54587 | D |
| matrix-remodelling associated 8 | | |
| Myef2 | 50804 | D |
| myelin expression factor 2 | | |
| Mylip | 29116 | I |
| myosin regulatory light chain interacting protein | | |
| Myo6 | 4646 | D |
| myosin VI | | |
| Myo1E | 4643 | D |
| myosin IE | | |
| Myom2 | 9172 | I |
| myomesin (M-protein) 2, 165 kDa | | |
| Naip | 4671 | D |
| similar to Occludin | | |
| Nap1l3 | 4675 | D |
| nucleosome assembly protein 1-like 3 | | |
| Nedd1 | 121441 | D |
| neural precursor cell expressed, developmentally | | |
| down-regulated 1 | | |
| Nenf | 29937 | D |
| neuron derived neurotrophic factor | | |
| Nfe2l3 | 9603 | D |
| nuclear factor (erythroid-derived 2)-like 3 | | |
| Nfib | 4781 | D |
| nuclear factor I/B | | |
| Nfix | 4784 | I |
| nuclear factor I/X | | |
| Nfkbie | 4794 | D |
| nuclear factor of kappa light polypeptide gene | | |
| enhancer in B-cells inhibitor, epsilon | | |
| Nhedc1 | 150159 | D |
| Na+/H+ exchanger domain containing 1 | | |
| Nipsnap3b | 55335 | D |
| nipsnap homolog 3B (C. elegans) | | |
| Nln | 57486 | I |
| neurolysin (metallopeptidase M3 family) | | |
| Nr4a2 | 4929 | D |
| nuclear receptor subfamily 4, group A, member 2 | | |
| Nrbp2 | 340371 | D |
| nuclear receptor binding protein 2 | | |
| Nt5m | 56953 | I |
| 5′,3′-nucleotidase, mitochondrial | | |
| Nupl1 | 9818 | D (HT) |
| nucleoporin like 1 | | |
| Ocm | 654231 | I |
| oncomodulin | | |
| Or7e104p | 81137 | I |
| olfactory receptor, family 7, subfamily E, member 104 | | |
| pseudogene | | |
| Orc4l | 5000 | D |
| origin recognition complex, subunit 4-like (S. cerevisiae) | | |
| Osgepl1 | 64172 | D |
| Osialoglycoprotein endopeptidase-like 1 | | |
| Otud7b | 56957 | D |
| OTU domain containing 7B | | |
| Pabpc1l2b/Rp11-493k23.2 | 645974 | D |
| similar to poly(A) binding protein, cytoplasmic 1 | | |
| Pafah1b1 | 5048 | D |
| platelet-activating factor acetylhydrolase, isoform 1b, | | |
| beta1 subunit | | |
| Pard6b | 84612 | D |
| par-6 partitioning defective 6 homolog beta (C. elegans) | | |
| Parp2 | 10038 | D |
| poly (ADP-ribose) polymerase family, member 2 | | |
| Pawr | 5074 | D |
| PRKC, apoptosis, WT1, regulator | | |
| Pbrm1 | 55193 | I |
| polybromo 1 | | |
| Pcsk5 | 5125 | D |
| proprotein convertase subtilisin/kexin type 5 | | |
| Pde4dip | 9659 | D |
| phosphodiesterase 4D interacting protein | | |
| (myomegalin) | | |
| Pde6b | 50940 | D |
| phosphodiesterase 6B, cGMP-specific, rod, beta | | |
| (congenital stationary night blindness 3, autosomal | | |
| dominant) | | |
| Pde6d | 5147 | D |
| phosphodiesterase 6D, cGMP-specific, rod, delta | | |
| Pde9a | 5152 | I |
| phosphodiesterase 9A | | |
| Pex11a | 8800 | D |
| Peroxisomal biogenesis factor 11A | | |
| Pex6 | 5190 | I |
| peroxisomal biogenesis factor 6 | | |
| Pgap1 | 80055 | D |
| GPI deacylase | | |
| Phlda2 | 7262 | I |
| pleckstrin homology-like domain, family A, member 2 | | |
| Phtf1 | 10745 | D |
| Putative homeodomain transcription factor 1 | | |
| Pik3ip1 | 113791 | I |
| phosphoinositide-3-kinase interacting protein 1 | | |
| Piwil4 | 143689 | D |
| piwi-like homolog 4 (Drosophila) | | |
| Pknox2 | 63876 | D |
| PBX/knotted 1 homeobox 2 | | |
| Plce1 | 51196 | D |
| phospholipase C, epsilon 1 | | |
| Plekha8 | 84725 | D |
| Pleckstrin homology domain containing, family A | | |
| (phosphoinositide binding specific) member 8 | | |
| Plekhk1 | 219790 | D |
| pleckstrin homology domain containing, family K | | |
| member 1 | | |
| Plxnd1 | 23129 | I |
| Plexin D1 | | |
| Pmp22 | 5376 | D |
| peripheral myelin protein 22 | | |
| Pms2l1 | 5379 | D |
| postmeiotic segregation increased 2-like 1 | | |
| Ppara | 5465 | I |
| peroxisome proliferator-activated receptor alpha | | |
| Ppard | 5467 | D |
| peroxisome proliferator-activated receptor delta | | |
| Ppp1r9a | 55607 | D |
| protein phosphatase 1, regulatory (inhibitor) subunit | | |
| 9A | | |
| Prr14 | 78994 | D |
| proline rich 14 | | |
| Prss23 | 11098 | D |
| protease, serine, 23 | | |
| Psg6 | 5675 | D |
| pregnancy specific beta-1-glycoprotein 6 | | |
| Psmd9 | 5715 | D |
| proteasome 26S subunit, non-ATPase, 9 | | |
| Ptcd1 | 26024 | D |
| pentatricopeptide repeat domain 1 | | |
| Ptprm | 5797 | D |
| protein tyrosine phosphatase, receptor type, M | | |
| Pus10/Flj32312 | 150962 | D |
| hypothetical protein FLJ32312 | | |
| Pus7l | 83448 | D |
| pseudouridylate synthase 7 homolog (S. cerevisiae)- | | |
| like | | |
| Qrsl1 | 55278 | D |
| Glutaminyl-tRNA synthase (glutamine-hydrolyzing)- | | |
| like 1 | | |
| Rad18 | 56852 | D |
| RAD18 homolog (S. cerevisiae) | | |
| Rad23b | 5887 | D |
| RAD23 homolog B (S. cerevisiae) | | |
| Rad51c | 5889 | D |
| RAD51 homolog C (S. cerevisiae) | | |
| Rad54b | 25788 | D |
| RAD54 homolog B (S. cerevisiae) | | |
| Rad54l2 | 23132 | D |
| RAD54-like 2 (S. cerevisiae) | | |
| Ralgps1 | 9649 | D |
| Ral GEF with PH domain and SH3 binding motif 1 | | |
| Ralgps2 | 55103 | D |
| Ral GEF with PH domain and SH3 binding motif 2 | | |
| Rap1gds1 | 5910 | D |
| RAP1, GTP-GDP dissociation stimulator 1 | | |
| Rasgrf2 | 5924 | D |
| Ras protein-specific guanine nucleotide-releasing | | |
| factor 2 | | |
| Rbm45/Drb1 | 129831 | D |
| Developmentally regulated RNA-binding protein 1 | | |
| Rdx | 5962 | D |
| Radixin | | |
| Rfpl2 | 10739 | D |
| ret finger protein-like 2 | | |
| Rgs9bp/Rgs9 | 388531 | D |
| regulator of G-protein signaling 9 | | |
| Rnf2 | 6045 | D |
| ring finger protein 2 | | |
| Rpap1 | 26015 | D |
| RNA polymerase II associated protein 1 | | |
| Rpl10 | 6134 | I |
| ribosomal protein, large, 10 | | |
| Rrp1 | 8568 | I |
| ribosomal RNA processing 1 homolog (S. cerevisiae) | | |
| Rps3a | 6189 | D |
| ribosomal protein S3A | | |
| Rps16 | 6217 | D |
| ribosomal protein S16 | | |
| Rttn | 25914 | D |
| rotatin | | |
| Rundc2c | 440352 | D |
| RUN domain containing 2C | | |
| Sccpdh | 51097 | D |
| saccharopine dehydrogenase (putative) | | |
| Sclt1 | 132320 | D |
| sodium channel and clathrin linker 1 | | |
| Scoc | 60592 | D |
| short coiled-coil protein | | |
| Sdccag8 | 10806 | D |
| serologically defined colon cancer antigen 8 | | |
| Sdhb | 6390 | D |
| succinate dehydrogenase complex, subunit B, iron | | |
| sulfur (lp) | | |
| Sec22a | 26984 | D |
| SEC22 vesicle trafficking protein homolog C (S. cerevisiae) | | |
| Sec23ip | 11196 | D |
| SEC23 interacting protein | | |
| Sephs1 | 22929 | D |
| selenophosphate synthetase 1 | | |
| Sept2 | 4735 | I |
| septin 2 | | |
| Sept8 | 23176 | D |
| septin 8 | | |
| Setd4 | 54093 | D |
| SET domain containing 4 | | |
| Setd8 | 387893 | D |
| SET domain containing (lysine methyltransferase) 8 | | |
| Sfrs10 | 6434 | D |
| splicing factor, arginine/serine-rich 10 (transformer 2 | | |
| homolog, Drosophila) | | |
| Sfrs2ip | 9169 | D |
| splicing factor, arginine/serine-rich 2, interacting | | |
| protein | | |
| Sfrs4 | 6429 | I |
| splicing factor, arginine/serine-rich 4 | | |
| Sgta | 6449 | D |
| small glutamine-rich tetratricopeptide repeat (TPR)- | | |
| containing, alpha | | |
| Siah1 | 6477 | I |
| seven in absentia homolog 1 (Drosophila) | | |
| Sipa1l3 | 23094 | D |
| signal-induced proliferation-associated 1 like 3 | | |
| Sla2 | 84174 | I |
| Src-like-adaptor 2 | | |
| Slc16a1 | 6566 | D |
| solute carrier family 16 (monocarboxylic acid | | |
| transporters), member 1 | | |
| Slc18a2 | 6571 | D |
| solute carrier family 18 (vesicular monoamine), | | |
| member 2 | | |
| Slc19a2 | 10560 | D |
| solute carrier family 19 (thiamine transporter), member 2 | | |
| Slc25a23 | 79085 | D (HT) |
| solute carrier family 25 (mitochondrial carrier; | | |
| phosphate carrier), member 23 | | |
| Slc2a13 | 114134 | D |
| solute carrier family 2 (facilitated glucose transporter), | | |
| member 13 | | |
| Slc30a5 | 64924 | D |
| solute carrier family 30 (zinc transporter), member 5 | | |
| Slc39a8 | 64116 | D |
| solute carrier family 39 (zinc transporter), member 8 | | |
| Slc45a3 | 85414 | I |
| solute carrier family 45, member 3 | | |
| Smek2 | 57223 | D |
| AW011752 KIAA1387 protein | | |
| Smg5 | 23381 | D |
| Smg-5 homolog, nonsense mediated mRNA decay | | |
| factor (C. elegans) | | |
| Sorbs3 | 10174 | D |
| sorbin and SH3 domain containing 3 | | |
| Spag10 | 54740 | I |
| sperm associated antigen 10 | | |
| Sphk2 | 56848 | D |
| sphingosine kinase 2 | | |
| Ssh3 | 54961 | D |
| slingshot homolog 3 (Drosophila) | | |
| St3gal3 | 6487 | D |
| ST3 beta-galactoside alpha-2,3-sialyltransferase 3 | | |
| St8sia1 | 6489 | D |
| ST8 alpha-N-acetyl-neuraminide alpha-2,8- | | |
| sialyltransferase 1 | | |
| Stag3 | 10734 | D |
| stromal antigen 3 | | |
| Steap3 | 55240 | D |
| STEAP family member 3 | | |
| Strbp | 55342 | D |
| Spermatid perinuclear RNA binding protein | | |
| Stx6 | 10228 | D |
| syntaxin 6 | | |
| Suhw2 | 140883 | D |
| suppressor of hairy wing homolog 2 (Drosophila) | | |
| Sycp2 | 10388 | D |
| synaptonemal complex protein 2 | | |
| Syne1 | 23345 | D |
| synaptic nuclear envelope 1 | | |
| Synpo | 11346 | D |
| synaptopodin | | |
| Tas2r14 | 50840 | D |
| Taste receptor, type 2, member 14 | | |
| Tbc1d24 | 57465 | D |
| TBC1 domain family, member 24 | | |
| Tc2n/Mtac2d1 | 123036 | I |
| membrane targeting (tandem) C2 domain containing 1 | | |
| Tdrkh | 11022 | D |
| tudor and KH domain containing | | |
| Tex261 | 113419 | I |
| testis expressed sequence 261 | | |
| Tfec | 22797 | D |
| transcription factor EC | | |
| Tgfb3 | 7043 | I |
| transforming growth factor, beta 3 | | |
| Tgm2 | 7052 | D |
| transglutaminase 2, C polypeptide | | |
| Thap9 | 79725 | D |
| THAP domain containing 9 | | |
| Thbs1 | 7057 | I |
| thrombospondin 1 | | |
| Tigd7 | 91151 | D |
| tigger transposable element derived 7 | | |
| Tjp3 | 27134 | D (HT) |
| tight junction protein 3 (zona occludens 3) | | |
| Tk1 | 7083 | I |
| thymidine kinase 1, soluble | | |
| Tlr9 | 54106 | D |
| toll-like receptor 9 | | |
| Tmem126b | 55863 | D |
| transmembrane protein 126B | | |
| Tmem169 | 92691 | D |
| transmembrane protein 169 | | |
| Tmem30b | 161291 | D |
| transmembrane protein 30B | | |
| Tmem41a | 90407 | D |
| transmembrane protein 41a | | |
| Tmprss6 | 164656 | D |
| transmembrane protease, serine 6 | | |
| Tmtc1 | 83857 | D |
| transmembrane and tetratricopeptide repeat | | |
| containing 1 | | |
| Tnfrsf11A | 8792 | D |
| tumor necrosis factor receptor superfamily, member | | |
| 11a, NFKB activator | | |
| Tnk1 | 8711 | D |
| tyrosine kinase, non-receptor, 1 | | |
| Top1mt | 116447 | D |
| topoisomerase (DNA) I, mitochondrial | | |
| Tpd52 | 7163 | D (HT) |
| tumor protein D52 | | |
| Tpp2 | 7174 | D |
| tripeptidyl peptidase II | | |
| Trabd | 80305 | D |
| TaB domain containing | | |
| Trim6 | 117854 | D |
| tripartite motif-containing 6 | | |
| Trip11 | 9321 | D |
| thyroid hormone receptor interactor 11 | | |
| Trove2 | 6738 | D |
| TROVE domain family, member 2 | | |
| Trpc1 | 7220 | D |
| transient receptor potential cation channel, subfamily | | |
| C, member 1 | | |
| Trpm7 | 54822 | I |
| transient receptor potential cation channel, subfamily | | |
| M, member 7 | | |
| Trspap1 | 54952 | I |
| tRNA selenocysteine associated protein 1 | | |
| Tshz2 | 128553 | D |
| Teashirt family zinc finger 2 | | |
| Ttc18 | 118491 | D |
| tetratricopeptide repeat domain 18 | | |
| Ttc30b | 150737 | D (HT) |
| tetratricopeptide repeat domain 30B | | |
| Txndc8 | 255220 | I |
| thioredoxin domain containing 8 | | |
| Ube2i | 7329 | I |
| ubiquitin-conjugating enzyme E2I | | |
| Ugt8 | 7368 | D |
| UDP glycosyltransferase 8 (UDP-galactose ceramide | | |
| galactosyltransferase) | | |
| Urg4 | 55665 | D |
| up-regulated gene 4 | | |
| Usp6nl | 9712 | D |
| USP6 N-terminal like | | |
| Usp7 | 7874 | I |
| Ubiquitin specific peptidase 7 (herpes virus- | | |
| associated) | | |
| Wdr20 | 91833 | D |
| WD repeat domain 20 | | |
| Wdr23 | 80344 | D |
| WD repeat domain 23 | | |
| Wdr34 | 89891 | D |
| WD repeat domain 34 | | |
| Wdr52 | 55779 | D |
| WD repeat domain 52 | | |
| Wdr71 | 80227 | D |
| WD repeat domain 71 | | |
| Wee1 | 7465 | D |
| WEE1 homolog (S. pombe) | | |
| Xpr1 | 9213 | D |
| xenotropic and polytropic retrovirus receptor | | |
| Zc3H12c | 85463 | D |
| zinc finger CCCH-type containing 12C | | |
| Zdhhc11 | 79844 | D |
| zinc finger, DHHC-type containing 11 | | |
| Zdhhc14 | 79683 | D |
| zinc finger, DHHC domain containing 14 | | |
| Zdhhc21 | 340481 | D |
| zinc finger, DHHC domain containing 21 | | |
| Zdhhc24 | 254359 | D |
| zinc finger, DHHC-type containing 24 | | |
| Zdhhc4 | 55146 | I |
| zinc finger, DHHC-type containing 4 | | |
| Zmiz2 | 83637 | I |
| zinc finger, MIZ-type containing 2 | | |
| Zmym5 | 9205 | D |
| zinc finger, MYM-type 5 | | |
| Znf10 | 7556 | D |
| zinc finger protein 10 | | |
| Znf169 | 169841 | I |
| zinc finger protein 169 | | |
| Znf204 | 7754 | D |
| zinc finger protein 204 | | |
| Znf236 | 7776 | D |
| zinc finger protein 236 | | |
| Znf24 | 7572 | D |
| zinc finger protein 24 | | |
| Znf318 | 24149 | D |
| zinc finger protein 318 | | |
| Znf492 | 57615 | D |
| zinc finger protein 492 | | |
| Znf502 | 91392 | D (HT) |
| zinc finger protein 502 | | |
| Znf540 | 163255 | D |
| zinc finger protein 540 | | |
| Znf557 | 79230 | D |
| zinc finger protein 557 | | |
| Znf569 | 148266 | D |
| zinc finger protein 569 | | |
| Znf585A | 199704 | D |
| zinc finger protein 585A | | |
| Znf608 | 57507 | D |
| zinc finger protein 608 | | |
| Znf614 | 80110 | D |
| zinc finger protein 614 | | |
| Znf624 | 57547 | D |
| zinc finger protein 624 | | |
| Znf667 | 63934 | D |
| zinc finger protein 667 | | |
| Znf684 | 127396 | D |
| zinc finger protein 684 | | |
| Znf710 | 374655 | D |
| zinc finger protein 710 | | |
| Znf711 | 7552 | D |
| zinc finger protein 711 | | |
| Znf718 | 255403 | D |
| zinc finger protein 718 | | |
| Znf793 | 390927 | I |
| zinc finger protein 793 | | |
| ZNF91 | 7644 | D |
| zinc finger protein 91 | | |
| Zscan5 | 79149 | D |
| zinc finger and SCAN domain containing 5 | | |

All-low and high threshold candidate genes.
Known genes shown only.
For human blood data:
I - increased in high mood (mania);
D - decreased in high mood (mania)/increased in low mood (depression).
(HT)—High threshold.

The nucleic acid sequences provided herein represent a region or a segment of the genes listed in one or more of the tables. The completed nucleic acid sequences for the genes listed in the tables are readily obtained from a public database (e.g., NCBI) using the gene identification (Gene ID) number and the gene names provided in the tables. Expression profiles of the genes listed in the tables are performed using either oligos, regions or segments of the genes or full or partial cDNA sequences, ESTs in a microarray format. Similarly, the presence or absence of the protein products or peptide fragments thereof of the genes listed in the tables are also analyzed for predictive and dignostic purposes. Antibodies to the protein or peptides are placed in an array format for serial or parallel expression profiling.

Mbp myelin basic protein Entrez Gene ID No. 4155

(SEQ ID NO: 1)

gaagataaccggctcattcacttcctcccagaagacgcgtggtagcgagt
aggcacaggcgtgcacctgctcccgaattactcaccgagacacacgggct
gagcagacggcccctgtgatggagacaaagagctcttctgaccatatcct
tcttaacacccgctggcatctcctttcgcgcctccctccctaacctactg
acccaccttttgattttagcgcacctgtgattgataggccttccaaagag
tcccacgctggcatcaccctccccgaggacggagatgaggagtagtcagc
gtgatgccaaaacgcgtcttcttaatccaattctaattctgaatgtttcg
tgtgggcttaataccatgtctattaatatatagcctcgatgatgagagag
ttacaaagaacaaaactccagacacaaacctccaaatttttcagcagaag
cactctgcgtcgctgagctgaggtcggctctgcgatccatacgtggccgc
acccacacagcacgtgctgtgacgatggctgaac
Edg2 Endothelial differentiation, lysophosphatidic
acid G-protein-coupled receptor, 2 Entrez Gene ID
No. 1902,

(SEQ ID NO: 2)

aatgagcgccacctttaggcagatcctctgctgccagcgcagtgagaacc
ccaccggccccacagaaagctcagaccgctcggcttcctccctcaaccac
accatcttggctggagttcacagcaatgaccactctgtggtttagaacgg
aaactgagatgaggaaccagccgtcctctcttggaggataaacagcctcc
ccctacccaattgccagggcaaggtggggtgtgagagaggagaaaagtca
actcatgtacttaaacactaaccaatgacagtatttgttcctggacccca
caagacttgatatatattgaaaattagcttatgtgacaaccctcatcttg
atccccatcccttctgaaagtaggaagttggagctcttgcaatggaattc
aagaacagactctggagtgtccattta
Fgfr1 fibroblast growth factor receptor 1 Entrez
Gene ID No. 2260,

(SEQ ID NO: 3)

ctcctctccacctgctggtgagaggtgcaaagaggcagatctttgctgcc
agccacttcatcccctcccagatgttggaccaacacccctccctgccacc
aggcactgcctgagggcagggagtgggagccaatgaacaggcatgcaagt
gagagcttcctgagctttctcctgtcggtttggtctgttttgccttcacc
cataagcccctcgcactctggtggcaggtgcttgtcctcagggctacagc
agtagggaggtcagtgcttcgagccacgattgaaggtgacctctgcccca
gataggtggtgccagtggcttattaattccgatactagtttgctttgctg
accaaatgcctggtaccagaggatggtgaggcgaaggcaggttgggggca
gtgttgtggcctggggccagccaacactggggctctgtatatagctatga
agaaaacacaaagttgataaatctgagtatatatttacatgtctttttaa
aagggtcgttaccagagatttacccatcg
Fzd3 frizzled homolog 3 (Drosophila) Entrez Gene
ID No. 7976

(SEQ ID NO: 4)

aatcctaaatgtgtggtgactgctttgtagtgaactttcatatactataa
actagttgtgagataacattctggtagctcagttaataaaacaatttcag
aattaaagaaattttctatgcaaggtttacttctcagatgaacagtagga
ctttgtagttttatttccactaagtgaaaaaagaactgtgtttttaaact
gtaggagaatttaataaatcagcaagggtattttagctaatagaataaaa
gtgcaacagaagaatttgattagtctatgaaaggttctcttaaaattcta
tcgaaataatcttcatgcagagatattcagggtttggattagcagtggaa
taaagagatgggcattgtttcccctataattgtgctgtttttataacttt
tgtaaatattactttttctggctgtgtttttataacttatccatatgcat
gatggaaaaattttaatttgtagccatcttttcccat
Mag myelin-associated glycoprotein Entrez Gene ID
No. 4099

(SEQ ID NO: 5)

tttggcgtcgtcctcaagttatattagaatcgtgtcctcccggctttggc
caacttactattctaggacttgattccttcattcagtcacaatttattga
gcaccgactttgcatcaacctcttgctgaagataacagtgctgacaatat
acagccctgccctcagagcttatatagtagaggagaaaaagtgaacccat
aatatacagtcagtagcgagtatttactaagtactttctatttgcgaggc
cctgataaaagtactgtcctggccaggcgcggtggctcacgcctgtaatt
ccagcactttgggaggtcgaggtgggcagatcacctaaggtcaggagttc
gagatcagcctggctaacatggggaaaccccgtctctactaaaaatggaa
aaattagctgggcatggtggcgggcgcctgtaatcccagctactcgggag
gctgagacaggagaatgacttgaacccaggagttgcagtggccaagataa
gatagcgccattgtactcc
Pmp22 peripheral myelin protein Entrez Gene ID No.
225376

(SEQ ID NO: 6)

tgtgaagctttacgcgcacacggacaaaatgcccaaactggagcccttgc
aaaaacacggcttgtggcattggcatacttgcccttacaggtggagtatc
ttcgtcacacatctaaatgagaaatcagtgacaacaagtctttgaaatgg
tgctatggatttaccattccttattatcactaatcatctaaacaactcac
tggaaatccaattaacaattttacaacataagatagaatggagacctgaa
taattctgtgtaatataaatggtttataactgcttttgtacctagctagg
ctgctattattactataatgagtaaatcataaagccttcatcactcccac
atttttcttacggtcggagcatcagaacaagcgtctagactccttgggac
cgtgagttcctagagcttggctgggtctaggctgttctgtgcctccaagg
actgtctggcaatgacttgtattggccaccaactgtagatgtatatatgg
tgcccttctgatgctaagactccagaccttttgt
Ugt8 UDP glycosyltransferase 8 (UDP-galactose
ceramide galactosyltransferase) Entrez Gene ID No.
7368

(SEQ ID NO: 7)

attccatgtcattctgtttacttagcacttgcactacccttgtnggttga
gtgtatgctttatttgtttctagtttgaaatcccacatctgatagctgag
agtaggcaaatacaacatttacctaatgtcattcactaacatggaagagt
tgtgaaaattctagagtgctgtaaatccttggcatacactatgacaaaca
acttcattactctcccaccaggagctgctctcctgcacttagaaataatg
tcacaagtagttttctaatgtacaatgcagacaaatgtactgctctctga
atacttgaagaaatggtattatacatacatagaaacttattagttatacc
ttttcacaatcttattacgatgttgccgttaaaagggaaaaaagacacag
gcaatgaatggtgggatagtaagaggacttagagtgtatgaatgagttga
ttttacttttttggaatttgattaagttgacagtaggcactgattggatg
attaaacataagttaatctccactgtgat
Erbb3 Neuregulin receptor (v-erb-a erythroblastic
leukemia viral oncogene homolog 4 (avian)) Entrez
Gene ID No. 2065

(SEQ ID NO: 8)

cttatggtatgtagccagctgtgcactttcttctctttcccaaccccagg
aaaggttttccttattttgtgtgctttcccagtcccattcctcagcttct
tcacaggcactcctggagatatgaaggattactctccatatcccttcctc
tcaggctcttgactacttggaactaggctcttatgtgtgcctttgtttcc
catcagactgtcaagaagaggaaagggaggaaacctagcagaggaaagtg
taattttggtttatgactcttaaccccctagaaagacagaagcttaaaat
ctgtgaagaaagaggttaggagtagatattgattactatcataattcagc
acttaactatgagccaggcatcatactaaacttcacctacattatctcac
t
Igfbp4 insulin-like growth factor binding protein
Entrez Gene ID No. 43487

(SEQ ID NO: 9)

agagacatgtaccttgaccatcgtccttcctctcaagctagcccagaggg
tgggagcctaaggaagcgtggggtagcagatggagtaatggtcacgaggt
ccagacccactcccaaagctcagacttgccaggctccctttctcttcttc
cccaggtccttcctttaggtctggttgttgcaccatctgcttggttggct
ggcagctgagagccctgctgtgggagagcgaagggggtcaaaggaagact
tgaagcacagagggctagggaggtggggtacatttctctgagcagtcagg
gtgggaagaaagaatgcaagagtggactgaatgtgcctaatggagaagac
ccacgtgctaggggatgaggggcttcctgggtcctgttcccctaccccat
ttgtggtcacagccatgaagtcaccgggatgaacctatccttccagtggc
tcgctccctgtagctctgcctccctctccatatctccttcccctacacct
ccctccccacacctccctactcccctgggcatcttctggcttgactggat
gg
Igfbp6 insulin-like growth factor binding protein
Entrez Gene ID No. 63489

(SEQ ID NO: 10)

gcgcgcctgctgttgcagaggagaatcctaaggagagtaaaccccaagca
ggcactgcccgcccacaggatgtgaaccgcagagaccaacagaggaatcc
aggcacctctaccacgccctcccagcccaattctgcgggtgtccaagaca
ctgagatgggcccatgccgtagacatctggactcagtgctgcagcaactc
cagactgaggtctaccgaggggctcaaacactctacgtgcccaattgtga
ccatcgaggcttctaccggaagcggcagtgccgctcctcccaggggcagc
gccgaggtccctgctggtgtgtggatcggatgggcaagtccctgccaggg
tctccagatggcaatggaagctcctcctgccccactgggagtagcggcta
aagctgggggatagaggggctgcagggccactggaaggaacatggagctg
tcatcactcaac
Pde6 dphosphodiesterase 6D, cGMP-specific, rod,
delta Entrez Gene ID No. 5147

(SEQ IDNO: 11)

gaagcaacgttttcatctgattggaaataccgtattgctgaaaagaagaa
aggcctttttaatggcttttgaacaaagcagaaaagtttgagcttctcac
cttcagtcttagctcttgaacctgttgagaaagaggataagagacaaata
cggaaaagagtttcagaaagcagaatctgtgtcagcccactggaaggaaa
agcgaatcaaccgattcagtgatgttagtgcatccagaaacaggcttttg
ggaaaagcttgacctgagctgattaaatcctgaagcacaanggaagcagc
cacatcaaaaagttagcatgagagcagtggcgtgctcatcctctggtagc
ctttactgggcatttgtggagtaagagagaaaagaaaagcaggaatgtta
agatatgctactaccttcaggaaa
Ptprm protein tyrosine phosphatase, receptor type,
M Entrez Gene ID No. 5797

(SEQ ID NO: 12)

gagcagcgtagacagctggtaaactgaagagcacaactatattcttatga
aggaatttgtacctttggggtattattttgtggcccgtgaccctcgttat
tgttacagctgagtgtatgtttttgttctgtggagaatgctatctggcat
tatggtaatatattattttaggtaatatttgtactttaacatgttgcata
atatatgcttatgtagctttccaggactaacagataaatgtgtaatgaac
aaagatatgttgtatgagtcgtcgtttctgtcagatttgtattgtttcca
agggaaaagcttgggggaggactcagttcacaaaatgcaaaactcaacga
tcagattcacggacccagagcttttccatgtgt
Atp2c1 ATPase, Ca++-sequestering Entrez Gene ID
No. 27032

(SEQ ID NO: 13)

tttttctcttctggcttcataaatgccttgctgtataaattgaaatattg
atactgaactgtctttttaatgatgacctaactttattcaacccatcgga
atttactttttccctgaaataagatcttttccactggtctactacctgac
cataaacatgtctgcatttgaattctctaaaccctaaatctgtgtctatg
Atxn1 Ataxin Entrez Gene ID No. 16310

(SEQ ID NO: 14)

aaacagagcagccacgggctcgaaccgaatccccgccgtccttagaaacg
gatttttttttgttttgttttgttttctggcagagtctcgatcaccaccc
tactnccacccccactaaggttcttgctcaatctccctagaaaacctgaa
ttgtttcatccctttcagtcagccccctacgtggtctgaaacaaaatgaa
agcacaagccacggagtttaagaaggcagcctgaaggcggggggctgaag
aggggtcggggcgctgcagagtcagccaagtagccaaggaagggccccct
ccgcgtcgcgacggccctcgccccccgcccggcgcgcgcgcgcacaaata
cacacacacagtcactcacacactcactcacactcacgccgcgctccgac
accgcctcacctctcgctccgcccgtccggcccccttccctgccccctgc
gggaccggcctgcgcgcagcactggaaccacgtaggaggaggcggcgg
Btg1 B-cell translocation gene 1, anti-prolifera-
tive Entrez Gene ID No. 694

(SEQ ID NO: 15)

gaaggtatacagactgccaacattttagacttatctctcagtctctgcca
ctgaacttttatatatggctgctatcaaaatataaccggtttattttcat
atttggaactaatataacagtatcacaaaatcttttacagtaagatagtt
tgtaataccagccgacccagctgcttaactgagtccttaaatcatttaat
atatgggactgtaaatagagaaatctgtacattannagatctgatttctg
gttatgcctatagatctttattttctttatccctatagatcattttcttc
tgatgttaagtgttatatttttgaaatgctcctaaacaagtaagccttag
attgtattaatcctgaggattgataccatttcctcaacactttgtggagt
atgattgacaccagtttttttttgactgcaggtttaacttggcttatcac
ttttcgtattgctcagtatgtccaag
C6orf182 chromosome 6 open reading frame Entrez
Gene ID No. 182285753

(SEQ ID NO: 16)

cacagttttgtgaagaagcctttaatccaagttttatgagacagtaggaa
cctatagctacttaatttttaggagacagtaattcagttgcagaagcttt
cctctgctattcccattctcttttacaaaactagttttttttaaaaaatc
aatgatcaattttatcttactactttataacttttgctgacttttattct
ttgcattgtatgtaatgtccatcagtataaattgagactgtgaatttcta
ggacccacaaaagtggtattttttttttgtacaagaaagtatagaggaag
aggaagctgggcattaaattacctcatccagcag
Dicer1 Dicer1, Dcr-1 homolog (Drosophila) Entrez
Gene ID No. 23405

(SEQ ID NO: 17)

tatcactggtaaggagcccacgaccagacttcatttctgggaaaatgaga
ctttgtgttgatctcatcgtgttggccttgtaaaagtgatctatgcatgt
acagtgttcatgcttaatattcaagggatggggcggggaacaaaaggaat
agaaagaattcttttccttgttatttggggagcacgtattgctttataac
tttggttgttgggagtatggctatcatataccctcatcagtgtcatttta
tatctgcctaattagagaaattttaaccttagtattttgatgtgttttcc
ccattttatcctccgcaaatatctttctcttgcccattcagtgctgcttt
tggtttttgatttagttgtatattctggatgtatttccacagccttttat
tgttcttcc
Dnajc6 DnaJ (Hsp40) homolog, subfamily C, member
Entrez Gene ID No. 69829

(SEQ ID NO: 18)

gatccagtatgtgcttgtcttatttaaaattggaatgtgagacatgttgc
tgtgacctgtttttctttctcattcacatttgtagatattgtgtgaacta
cagtatataatgataacaattaaaaggatattctgtggatgtcacgtatt
ttgaaatgatagaactacattagctttgtatcatgtttggataattcatc
aatgttcacagtttaaaacatcattaaacattatgtaattacaatgagaa
agaatcttacttaaatttggagattttcccccacatctcttttccggata
cattataattctggacccctatttatctcaaaactcttaatatatgcaga
ccaacaggtctttgcattccttttaaataactggttgtgacaaagcttgt
tgttgatcagattcactg
Ednrb endothelin receptor type B Entrez Gene ID
No. 1910

(SEQ ID NO: 19)

aactgctttaagtcatgcttatgctgctggtgccagtcatttgaagaaaa
acagtccttggaggaaaagcagtcgtgcttaaagttcaaagctaatgatc
acggatatgacaacttccgttccagtaataaatacagctcatcttgaaa
Elovl5 ELOVL family member 5, elongation of long
chain fatty acids (yeast) Entrez Gene ID No. 60481

(SEQ ID NO: 20)

tcgaggtatcagcagctctgtcctcagaatgggtcccccacttcacacag
ttgtaggatggctacagcagctccaagcagcacattcagaggaagaagaa
aaaatgtttcatttgtgtggttttaagcataaagaagttgtttcccagag
ctctttgccagctgtcatccctcaaaactcactggccacaattgcttcac
atgcccctgcctgaaccagccaccagagggggttaggaccaccacagatt
gactagatcctaaggattctctacctggggctggagttcgggtcaggtgc
ccgggaagcactgtgcggtgtgggaggatg
Gnal guanine nucleotide binding protein, alpha
stimulating, olfactory type Entrez Gene ID No.
2774

(SEQ ID NO: 21)

tgttgttgtccctattgctggtttattacactgtacagaccacaaaatgt
aatattcttttgtataactactaaagaaaaatccttgtagancnnnnnnc
cttcaccatggctatctatacctgtacatgaaatgtgtttgtattgtgct
gaagngcttaatgtcaacattacctgctgnttactctgaaaaaaggaatg
aatggtagctntagaatttaggatattttatcaggttggcactttataaa
atactccctgatttaaaaaattgtaagttatacacgttaatcatccacat
tctatcgacaatgtaccaacatcacaagctgttgcaaccacctgnctgtt
acttctctgagctgttaaaancctggaacttcaatttcaggggggcacaa
at
Klf5 Kruppel-like factor Entrez Gene ID No. 5688

(SEQ ID NO: 22)

ttacagtgcagtttagttaatctattaatactgactcagtgtctgccttt
aaatataaatgatatgttgaaaacttaaggaagcaaatgctacatatatg
caatataaaatagtaatgtgatgctgatgctgttaaccaaagggcagaat
aaataagcaaaatgccaaaaggggtcttaattgaaatgaaaatttaattt
tgtttttaaaatattgtttatctttatttatttgggggtaatattgtaag
ttttttagaagacaattttcataacttgataaattatagttttgtttgtt
agaaaagtagctcttaaaagatgtaaatagatgacaaacgatgtaaataa
ttttgtaagaggcttcaaaatgtttatacgtggaaacacacctacatgaa
aagcagaaatcggttgctgttttgcttctttttccctcttatttttgtat
tgtggtcatttcctatgcaaataatggagcaaacagctgtatagttgtag
aat
Lin7a lin 7 homolog a (C. elegans) Entrez Gene ID
No. 8825

(SEQ ID NO: 23)

aatggaccaattttagctcaacttttagtttgttagaagcaagtgtagga
actctagcactgtagtttttaattattgcttgtatctattattattaatt
ccaacagagtataatgtatatttattctataaaatatatattatcagagt
gcatttgttacaacttaggttcttttcttaccaagtattaagnaatctag
taagagnaatactagncaaaggacctagnccctgtgnaacagnntctcgt
atgttatatacataaacccactctgc
Manea mannosidase, endo-alpha Entrez Gene ID No.
79694

(SEQ ID NO: 24)

gtgtttctgctttacagtgctgaattccatattttagaagctatgaaagt
ccttttatgaaaaagttactgattgcttctcagttattaggaaaacagtt
gtttcacaattattatgtagatatgatgcccaaatatcatttttagtata
tcttgtcgatctttaagttgttactattgtgttattcatgtctttaaatc
agataccaaatattttttaggaaagaaaaatgttattactgtcattaggt
tgtcttttaatactttaagttattttgacgaaaagtaatagagaaaattt
acttagcattttagattctagagacatggaaatgaaaattattttatgtc
tagagtaggtcctgaagtttggctttacattaagtttagcactgtatcag
aatgaagaaactaatattttacataaaaactaatactttcaattttttat
atagtaatatccccattttgtaaatgttagacttttatcatacctgtaa
Nupl1 nucleoporin like 19818

(SEQ ID NO: 25)

aaaataggcattgcatacacatatgcacacgtatgtgcacgtgccacaca
ttttttgtataatgttgggtttgattataaaagtgttgtcaaatgtttta
tttatctgcatntagcagtggttggcttttttgaattgaaatttttgcgc
attgatgcattgaaataaggaaaattatttatctctgagcactaaactta
tttttgcatatttctgtaatattgcagtccccagatccagaacatgggaa
gttagggaaaatgtgtgattttgtgttttgaattactgtcagaattacat
acacaattacaacaaactttttttaaaagacatttcattgtactgcaaaa
atctgaatatttatatttcttgtttttttctttatatgttttgcatttta
atatgttgagccactgg
Pde6b phosphodiesterase 6B, cGMP-specific, rod,
beta (congenital stationary night blindness 3,
autosomal dominant) Entrez Gene ID No. 5158

(SEQ ID NO: 26)

tcttttgaaaatgctggcctgctgcacctgttctcagaggggcatgagaa
aagaggattctcttcctagggctgtgggaagccccttgagatgttggaag
cagggcagggcaggagaacccagggagggcacagagctgtggacgagggc
tgggaaagccatcccgcctccccagggggtctccgaggagtgctgctgtg
gccaaaccaggggggccactttgtgctttctgtttaggtgatgggatgct
tctatctcctcagcaccccacaccaaatcccctgttattgccagatgatg
tgcctaatgatcaaattgctt
Slc25a23 solute carrier family 25 (mitochondrial
carrier; phosphate carrier), member Entrez Gene ID
No. 2379085

(SEQ ID NO: 27)

ctcccaccttctaggcgaatagtccccagagctgtgttcctccaaggggt
ccgaggaatcactcactcctggaggctggcaaggagacagtctgaggcca
gggacacatgaagggatgtccccaccccagcactatcagggcctccccag
gcttccagagttgaaagccaggagaaaatcggcaaagaccacccttccct
aaacccaagcacccaatgatgcaaaaaacaaaaacaaaaaaaaaccacca
aatccccaaattcattccagatctatttttctaccagagagaggagcaaa
gtcctcctcccctgcgcccttacattctgcacttcatagttggattctga
gcttaggatcatctggagaccccatggagggacttgga
Synpo synaptopodin Entrez Gene ID No. 11346

(SEQ ID NO: 28)

ggtgatgttagatctggaacccccaagtgaggctggagggagttaaggtc
agtatggaagatagggttgggacagggtgctttggaatgaaagagtgacc
ttagagggctccttgggcctcaggaatgctcctgctgctgtgaagatgag
aaggtgctcttactcagttaatgatgagtgactatatttaccaaagcccc
tacctgctgctgggtcccttgtagcacaggagactggggctaagggcccc
tcccagggaagggacaccatcaggcctctggctgaggcagtagcatagag
gatccatttctacctgcatttcccagaggactagcaggaggcagccttga
gaaaccggcagttcccaagccagcgcctggctgttctctcattgtcactg
ccctctccccaacctctcctctaacccactagagattgcctgtgtcctgc
c
Tgm2 transglutaminase 2, C polypeptide Entrez Gene
ID No. 7052

(SEQ ID NO: 29)

ccttcagaccccagttctaggggagaagagccctggacacccctgctcta
cccatgagcctgcccgctgcaatgcctagacttcccaacagccttagctg
ccagtgctggtcactaaccaacaaggttggncaccccagctaccccttct
ttgcagggctaaggcccccaaacatagcccctgccccggaggaagcttgg
ggaacccatgagttgtcagctttgactttatctcctgctctttctacatg
actgggcctcccttgggctggaagaattggggattctctattggaggtga
gatcacagcctccagggccccccaaatcccagggaaggacttggagagaa
tcatgctgttgcatttagaactttctgctttgcacaggaaagagtcacac
aattaatcaacatgtatattttctctatacatagagctctatttctctac
ggtttta
Tjp3 tight junction protein 3 (zona occludens 3)
Entrez Gene ID No. 27134

(SEQ ID NO: 30)

acacggatgtggatgatgagcccccggctccagccctggcccggtcctcg
gagcccgtgcaggcagatgagtcccagagcccgagggatcgtgggagaat
ctcggctcatcagggggcccaggtggacagccgccacccccagggacagt
ggcgacaggacagcatgcgaacctatgaacgggaagccctgaagaaaaag
tttangcgagtncatgatgcggagtcctccgatgaag
Tpd52 tumor protein D Entrez Gene ID No. 527163

(SEQ ID NO: 31)

agatgtgtccaaagagatccttacattaaggtcatttgtcagaatgatgt
ttgngtttgtttagagttggctgacctccaactcctggggtcaaggaatc
ctactgccttagcttcccaaatagctaggactataggcatgcacccggcc
atgtgttttatttatagctcttaaagcccagatgaagaaatcacattttt
gcccatagtgaagaaacatttggccattgattagtccttattttcagtga
ctgtcctgttttcattagattagagagaccctgtgtgggccacagttaat
ataaaccattatcacttttaagtaaacctgcacatcttagatttcataat
ttccttattgttctgactcaaaatgaactaagagcttttcactttttgtt
tgtaagttctcagagagtcgggtctgcaagtgcttt
Trpc1 transient receptor potential cation channel,
subfamily C, member Entrez Gene ID No. 17220

(SEQ ID NO: 32)

gggtgcaggtaacccttggtctgtaagcaccaccgatccagggatcattg
tctaaataggttactattgtttgtttcatcttgcttttgcatttttattt
tttaatttccaaattttaagtgttccctctttggggcaaattcttataaa
aatgtttattgtaaagttatatattttgtctacgatgggattatgcactt
cccaattgggattttacatctggatttttagtcattctaaaaaacaccta
attattaaaacatttatagagtgcctactgtatgcatgagttgagttgct
tctgaggtacattttga
Bclaf1 BCL2-associated transcription factor Entrez
Gene ID No. 19774

(SEQ ID NO: 33)

gttactagactataccttggcctttaaaaatgaatctcactgattaaaga
gaacaggcattattaggatagcattccaccacaactagaaacattcaaat
aatgtgtcttaattgtaatctgtatataggaaaatttttcctatggatat
ttttggtgttttaccacagtgaactgatttgtagcacttatgaagtgcag
aaggtaatattcttgaaaatagaaaaaggttgggtgagcaggctttaatg
cctttcccccaagaatatacatcgaatttttcttaatcttttggggttgg
ccagcttccagatttcattaataatgagctctgcctttaataaaagtaca
tgatcatagctacactgtatgtttaggtggtgtgaaatgatttataatca
cagcttgaactgtgtttgcttggtactgtca
Gosr2 golgi SNAP receptor complex member Entrez
Gene ID No. 29570

(SEQ ID NO: 34)

ccctgtgcctcagtgacatgtagatgactgactgccaatacttgtcacca
ttccctggaagcagctacctaggggaaacaagatgtagtgctattgccga
taacaagtaagattttccacactanannnnggtgtttctcttttctaaag
tgaggccagtgttatttcccgggagtgttcagtcttgaccctagtcactg
attttttctagttgttaatagagtggttggcttttaaggttcagagactg
tggcttggcacctgcgcccaggctttgtgggcctttgccccttagaaagt
agctgtaggcaaagatttgtgattttccaattacagtctcagctctagtt
ttagtatctctaattctttggttcccttctcttccctgaaatatattagc
acctgccagccaggccctcattttgcccagccagtgtgggcagatcccac
cgtggagacatctgtagtgtgtatgtccttgtaacactctgttttcaggg
actacaacctttttccttctgtgaccagccccggattcaggctgtac
Rdx radixin Entrez Gene ID No. 5962

(SEQ ID NO: 35)

ttaacactaattatcacgtctgacaaatgtgtatgtgtggtttcagttct
gtgtacattttaaaggataatggtgaacattttaatgggtttcccttgcc
ctttccatatttaacctatttccacattctctctcactcacattttctca
gtgtgcccttctcttatctgccatgtccatagccataattccaccatcat
acagatcaggcagtgtttaaaatgatggtaggtagcacagtggacagtct
ttgatcatcatgtagaatatggctatgaatcaggaaagagattagaacat
ttaataatgtatgtacagctggtgcttagtttttttttaatctaaattta
attaccttattggatatttgatatttggttatttaatcacagtcatcttt
aacagcttacactgattggtgttttatctcctgtgatcctttgatggctt
tttttgcctaccatttcacagaggtt
Wdr34 WD repeat domain Entrez Gene ID No. 3489891

(SEQ ID NO: 36)

cgctgggactgacgggcatgtccacctgtactccatgctgcaggcccctc
ccttgacttcgctgcagctctccctcaagtatctgtttgctgtgcgctgg
tccccagtgcggcccttggtttttgcagctgcctctgggaaaggtgacgt
gcagctgtttgatctccagaaaagctcccagaaacccacagttttgatca
agcaaacccaggatgaaagccctgtctactgtctggagttcaacagccag
cagactcagctcttggctgcgggcgatgcccagggcacagtgaaggtgtg
gcagctgagcacagagttcacggaacaagggccccgggaagctgaggacc
tggactgcctggcagcagaggtggcggcctgaggggtcccgggaggcggg
tgcaagccttcgctgtgccgagccttgtgtttctgacgcaagcca
Nefh neurofilament, heavy polypeptide 200 kDa
Entrez Gene ID No. 4744

(SEQ ID NO: 37)

ccccaggcgatggacaattatgatagcttatgtagctgaatgtgatacat
gccgaatgccacacgtaaacacttgactataaaaactgcccccctccttt
ccaaataagtgcatttattgcctctatgtgcaactgacagatgaccgcaa
taatgaatgagcagttagaaatacattatgcttgagatgtcttaacctat
tcccaaatgccttctgttttccaaaggagtggtcaagcccttgcccagag
ctctctattctggaagagcggtccaggtggggccgggcactggccactga
attatgccagggcgcactttccactggagttcactttcaattgcttctgt
gcaataaaaccaagtg
Bic BIC transcript

(SEQ ID NO: 38)

gggtaaataacatctgacagctaatgagatattttttccatacaagataa
aaagatttaatcaaaaaatttcatatttgaaatgaagtcccaaatctang
ttcaagttcaatagcttagccacataatacggttgtgcgagcagagaatc
tacctttccacttctaagcctgtttcttcctccatnnnatggggataata
ctttacaaggttgttgtgaggcttagatgagatagagaattattccataa
gataatcaagtgctacattaatgttatagttagattaatccaagaactag
tcaccctactttattagagaagagaaaagctaatgatttgatttgcagaa
tatttaaggtttggatttctatgcagtttttctaaataaccatcacttac
aaatatgtaaccaaacgtaattgttagtatatttaatgtaaacttgtttt
aacaactcttctcaacattttgtccaggttattcactgtaaccaaataaa
tctcatgagtctttagttgattta
Bivm basic, immunoglobulin-like variable motif
containing Entrez Gene ID No. 54841

(SEQ ID NO: 39)

atcatcatgatcgctcgacatcgatnnnnnnnnnnnnntttttttttttt
ttttttttttttnttnnaagtagaaaacaaaactttatttgatgaaatct
ttttaaaagttccagtatgaantaacaaaatcaacaacctacaaatctct
ttcagtcctttgcatttcaagcaaaatattctcttcagaaaaatgaccat
ttcataatatatatccccttctgtcg
Bnip1BCL2/adenovirus E1B 19 kDa interacting
protein 1 Entrez Gene ID No. 662

(SEQ ID NO: 40)

aaaccaccaaagagagcctggcccagacatccagtaccatcactgagagc
ctcatggggatcagcaggatgatggcccagcaggtccagcagagcgagga
ggccatgcagtctctagtcacttcttcacgaacgatcctggatgcaaatg
aagaatttaagtccatgtcgggcaccatccagctgggccggaagcttatc
acaaaatacaatcgccgggagctgacggacaagcttctcatcttccttgc
gctacgcctgtttcttgctacggtcctctatattgtgaaaaagcggctct
ttccatttttgtgagatcccaaaggtgccagttctggccctttcagctcc
tgtttcaggatctgtcctggttcctgagctctaggctgctaagctgagcc
acacacc
C8orf42 chromosome 8 open reading frame 42 Entrez
Gene ID No. 157695

(SEQ ID NO: 41)

gaaactctggaaatcacgtgtgtggggagatggggacgcttcccatgttg
tggggagctctgtggctgtgatggctgcagttgccgtgcctctgttggaa
cgcnaagtgcctgcaactcacgtcaatcatagaattgtgacgcacagttg
gcaaaatagttctttatgctatttctcaaaantttgaggacaaacccaga
ttgggattggaatatgcactgtaaatcaaatttttcttatctacaaagac
taatgtaaaaatgattttttcttctgtgcctgattaaattaactgtggtt
tttaatataaatatttattggtgtgctttgggagaaaaattatcttttct
tgaaagaanttatcaaagcaaatttattatcttcacaagttaatgggaga
atgtggttttgattctgggtgtttgaattgtgtaaacacacagcttcctt
gtg
Camk2d calcium/calmodulin-dependent protein kinase
II, delta Entrez Gene ID No. 817

(SEQ ID NO: 42)

atttcccttttacattcattatgcaaattcacnttctattcntttctcac
acactactagccagcctccccaaanaaggaaaagggaaaaaagtaagaaa
agaatggaacaaaagaaaaataagaaagcaaacgaaaggaacaaagaaac
aggataaagaaaagagatcacagatttgagaaagaaaaacaattcaattc
agcaaattcaccaaaacaatgtgaatatatcctaaagtgattaaactcag
aaatgatgtgaatttttccagtttacacagtttgaccaaaaacagcatgg
ctttatgtggtagcaaaccaactgattcttgcttctactttcataagtga
ttttgcccacatatcatcccactttaattgttaatcagcaaaactttcaa
tgaaaaatcatccattttaaccaggatcacacca
Dock9 Dedicator of cytokinesis 9 Entrez Gene ID
No. 23348

(SEQ ID NO: 43)

tctttgatcactgcctcttgattttttcctggatcattaagaggcttgaa
gaatactatgtagttgaaccagaggagtagtgtatgtcacatcctcactt
actccttaagccctttctcatggtcttggccctaaaacatattttcaggg
cttgtgacccagtgatcagtggtcacccttaaagtattacagatacgtgc
ctgttttacatgagaggtaactgtttatgtgtataagtcatcttaataaa
ataacatgaaatttattagctgaattgggtagatactgcttttctaagtt
gaacctaacttaagctgatgcagaaactgagtcagaaaagttgctataat
tttaaaatataagaagtaaaagtgaaatcttatgtagcatctttatctca
ttttggtttgtcagtataagtttctgatttcctttaagctctttactttt
agaaacgtgaatttacaatcccttatccaaaactgctg
Fam13a1 family with sequence similarity 13, member
A1 Entrez Gene ID No. 10144

(SEQ ID NO: 44)

gttagtggagttttactgttaatatcatcatgtccccctttgtgtttact
actgtctgaaattactgggatgtagaagcatatttcagtctgaaaattca
gccagcttattttggagaagttgtatcttgttcttgggcatgttagcctt
gtttttcatcccaatttga
Hist1h3b histone cluster 1, H3b Entrez Gene ID No.
8358

(SEQ ID NO: 45)

atggctcgtactaaacagacagctcggaaatccaccggcggtaaagcgcc
acgcaagcagctggctaccaaggctgctcgcaagagcgcgccggctaccg
gcggtgtgaaaaagcctcaccgttaccgtccgggtactgtggctctgcgt
gagatccgccgctaccaaaagtcgaccgagttgctgattcggaagctgcc
gttccagcgcctggtgcgagaaatcgcccaagacttcaagaccgatcttc
gcttccagagctctgcggtaatggcgctgcaggaggcttgtgaggcctac
ttggtagggctctttgaggacacaaacctttgcgccatccatgctaagcg
agtgactattatgcccaaagaca
Hrasls HRAS-like suppressor Entrez Gene ID No.
57110

(SEQ ID NO: 46)

agagcaggccaaccgagcgataagtaccgttgagtttgtgacagctgctg
ttggtgtcttctcattcctgggcttgtttccaaaaggacaaagagcaaaa
tactattaacaatttaccaaagagatattgatattgaaggaatttgggag
gaggaaaagaaacctggggtgaatacttattttcagtgcatcattactgt
tccagattcctatgatggatggc
Ibrdc2 IBR domain containing 3

(SEQ ID NO: 47)

cagccagtggctgtggtctacagaattgtttcatataaaatacgggtaga
gtggtagagtttcaaaactttcgtcatagatatctgggacctttctcagg
atctgtgttcacacagccaatagatttggaatcaggcctaagagtacaca
tggagggtaaatattaaagtgcgtattatgtacatctagaatccatgtga
cttgcagcctacctgtaatttctatccattgagcatgcatggatataccc
aatagtacacacaaaataaatgtttacttaagagccattctaaaaaannn
nnannnaaatggtttattgtaaatctgcctaaagattttttgcatattat
atatgtgaattttggttgtaagttcataacttacccaagggtatagactc
ataactcttttaaaacagtgcttagtacaatatcctgccatctctgtaaa
aacgctaattgataaccgagtcatttacatgttttcgaacacagaatagc
tcttttctcagcatcattattgctctttcagcatc
Kiaa1729 KIAA1729 protein Entrez Gene ID No. 85460

(SEQ ID NO: 48)

gattccagaatctctacctttaaacactatgttaccacttacttctcttc
aaattttattgagcattagatgtttccagtatttagaagtcaaatgcttc
gtttttaataggaacttacacagtcttttatgtttttttatagccctcaa
tgtcactgatgtggattctcccaaactcgatactttgtttgtttttatgt
ccccataataagtctttaagaaaacagggcaagtgagctcaaaatcaaaa
gaaaacccaccaacagtgaatgcattcagggctatttccaggtctttctt
ttgaagaaagataagactcagtccagagagcacatctgtgacacaccgtg
cctcttgcctttggtgcgtggcagtcatctttggctcatgctgtacatta
ttctac
Klf12 Kruppel-like factor 12 Entrez Gene ID No.
11278

(SEQ ID NO: 49)

gtttcgattctgttttgttcatctgttcgagcagaggggcagttgaagtc
tcgtcctggtctctgccctggcatggactggcacagaggtgttctgtagt
tgaataggaagagcctgtctaaaaaactactgccccacttcaaattgcag
tgttctgtcacctaggcatcatctcttcctgcccctagtatttgattaca
aggaaccaggggaaaaaaactttcttagacacactggcaccaaggtaaga
ggtggggctgcccaggcaaagtcagtgaacatgaaaactcagacaaagca
gagatggaaataatgcgcctcttgaggagaaaagcaataatgaataaaag
gactttcctacaataacttcactgaggactcacgttaccaattttcatac
ttactaaagggattgtaaaaaacaccccagcattttaggtgtcttggttc
catttacagcactgaggtaatctttctgctgtttgttgtcctgcttggtt
gagtacc
Loc253012 hypothetical protein LOC253012 Entrez
Gene ID No. 253012

(SEQ ID NO: 50)

ctcattattcctttacatgcagaatagaggcatttatgcaaattgaactg
cagtttttcagcatatacacaatgtcttgtgcaacagaaaaacatgttgg
ggaaatattcctcagtggagagtcgttctcatgctgacggggagaacgaa
agtgacaggggtttcctcataagttttgtatgaaatatctctacaaacct
caattagttctactctacactttcactatcatcaacactgagactatcct
gtctcacctacaaatgtggaaactttacattgttcgatttttcagcagac
t
Loc253039 hypothetical protein LOC253039 Entrez
Gene ID No. 253039

(SEQ ID NO: 51)

gaacttaagttcacacacccttgtactgcaggacggggaatggaacctag
gtcttcttatttttggttcagtgttaactcccattctctaagcagactgg
gcctgttattcaaactgccttcccataggtgcttccctgcttctctcctc
acccagagaaggacttacaaacagcttatcttncagaggttttgtgcctg
atagttatggaatgtgctggtttgagcagggaggatgtaaggggagggaa
tgctaaaaggctgtctacttagagtcaggtttcctgggtaagtccctgga
accccatccccttcccctttcttgagaccccaggacttgctccagtaact
gccaccctgtgcctttgcttcagggccatgctggataaggagctggctgc
ctctgtgaacatcctactcaaggcatcttcactgtgagttttgctgttgc
cattggaggggnngtggggggagtgtggggagtgctagggtcaggtcctg
gctggtgtaaagaac
Loc91431 prematurely terminated mRNA decay factor-
like Entrez Gene ID No. 91431

(SEQ ID NO: 52)

gactttcaccatcctgatattaaaactgtgcaggtgtccacagtagatgc
ttttcagggagctgaaaaggagatcattattctgtcctgtgtaaggacaa
gacaagtaggattcattgattcagaaaaaagaatgaatgttgcattgact
agaggaaagaggcatttgttgattgtgggaaatttagcctgtttgaggaa
aaatcaactttggggacgagtgatccaacactgcgaaggaagggaagatg
gattgcaacatgcaaaccagtatgaaccacagctgaaccatctccttaaa
gattatttt
Lrp16 LRP16 protein Entrez Gene ID No. 28992

(SEQ ID NO: 53)

gtaagactggcaaggccaagatcaccggcggctatcggctcccggccaag
tacgtcatccacacagtggggcccatcgcctacggggagcccagcgccag
ccaggctgccgagctccgcagctgctacctgagcagtctggacctgctgc
tggagcaccggctccgctcggtggcgttcccctgcatctccaccggcgtg
tttggctacccctgtgaggcggccgccgagatcgtgctggccacgctgcg
agagtggctggagcagcacaaggacaaggtggaccggctgatcatctgcg
tgttcctcgagaaggacgaggacatctaccggagccggctcccccactac
ttccccgtggcctgaggctcccgcagcccaccctgaccgggactgacccg
ccttcgggaccccgctcccagctctgagaggtcgccaaagcctgcagcct
ggcctgggcctggccaccccttctttccctccgcgccccgcccccgagga
gcctaataaagatctcgttgtggcaaaaa
Mical3 microtubule associated monoxygenase,
calponin and LIM domain containing 3 Entrez Gene
ID No. 57553

(SEQ ID NO: 54)

gggccccaagagcagactaggaacgcagggggctgctgctgccaggacnc
cacggagagccgggcacccgcctcacatgtctcctgtctggctccactga
gttagccgtttgagcccactcctatcttttggtggttagtgcatcttcag
ctcttttctgcaagacactggaacattcctaggctgtcccaaaaggagtt
ccaccatagcctttaaggtccgagcagggcaccaaggggttcacttttct
cccgagccattcagcttggggtgcctgcgggaggggcggacagccnagcc
ggcttcccggcggcggtacgagagcccaacaggagaggattagctgtgcc
aaggaacacgccactgctgcctgtctactgcccgccttctctccacttcc
atttttgcctttgtttttaacttgtgctcttgtgagttcttggtgtgttt
ctttgt
Mtap methylthioadenosine phosphorylase Entrez Gene
ID No. 4507

(SEQ ID NO: 55)

acaggactatttgccacgacatttcaaaggattccaagagagaatattgg
tgtccatgctgtgatgattcctcagctcctctcatctgatctccgtcctg
gcccccatgactttctttgcggtagttagggtgtggtatgtgccactgag
gcccacacctattggcaatttatagcactgatctgtcatcaataccactt
gctgtcttggatgtgaagatgatttttcctgcagggattccctctacaaa
attaaaaacactgggcatgtggaaataatattcacgctttaaattgtctt
ttctattcactacaccaggggtccccgacccctaggcaacagactgtggc
cctagtgtagtgaatagaaaagacaatttaaagcatgaatattatttcct
catgcccagtgtttttaattttggtactggtctgtggcttgttagaaa
Mtmr3 myotubularin related protein 3 Entrez Gene
ID No. 8897

(SEQ ID NO: 56)

agccgtcagctgtctgctatgagctgcagctctgcccacttacactcaag
gaacttgcaccacaagtggctgcatagccactcaggaaggccatctgcaa
ccagcagccccgaccagccttcccgcagccacctggacgatgatggcatg
tcagtgtacacagacacgatccaacagcgcctgcgtcagattgagtcagg
ccaccagcaggaagtagaaactttgaagaaacaagtccaggagctgaaga
gtcgcctggagagccagtacctgaccagctccctacactttaatggagac
tttggggatgaggtgatgacccgttggcttcctgaccacctggccgccca
ctgctatgcgtgcgacagtgccttctggcttgccagcaggaagcaccact
gcaggaattgtgggaacgtattctgctccagttgttgtaaccagaaggtt
ccagttcccagccagcagctctttgaacccagtcgagtatgcaagtcttg
ctatagcagcctacatcccacaagctccagcattgaccttgaactg
P2ry12 purinergic receptor P2Y, G-protein coupled
Entrez Gene ID No. 12 64805

(SEQ ID NO: 57)

aaatgtatatatatcctagtcccctaaccaaatcctgacctattgggata
cttataaaaatttaagtaagtgggatacacaaagaataataactattaac
ttttcattattagcaaaaacctaagggatttaaactaattgaaactgtat
ttgattggacttaattttttatgtttatttagaagataaagatttaaaga
agacctttacaataaagagaagaaatatcgaagtcattaaaataaggaga
cttacttttatgacattctaatactaaaaaatatagaaatatttccttaa
ttctagagaaactagttttactaattttttacaacttcaataataccatc
actgacacttacctttattaattagcttctagaaaatagctgctaattag
gttaatgaacattttaccttagtgnaaaaaaattaattaaatatgattac
aaagttgcacagcataactactgagaggaaagtgattgatctgtttgtaa
ttacttgt
Rad54b RAD54 homolog B (S. cerevisiae) Entrez Gene
ID No. 25788

(SEQ ID NO: 58)

gtaagcaaggtctttgtggggcagttgtcgacctcaccaagacatctgaa
catattcagttttcagtagaagaacttaaaaatttgttcacattacatga
aagttcagattgtgttactcatgatctgcttgactgtgagtgtacaggag
aagaagttcatacaggtgattcgttggaaaaattcattgtctctagagat
tgtcagcttggtccacatcaccagaaatctaactccctgaaacctctttc
tatgtcccagctgaagcaatggaaacatttttctggagatcatttaaatc
ttacagatccttttcttgaaagaataacagaaaatgtgtcattcattttt
cagaatataaccactcaagctactggcacatagtgaaagattacttctga
cattcca
Ralgps2 Ral GEF with PH domain and SH3 binding
motif 2 Entrez Gene ID No. 55103

(SEQ ID NO: 59)

acaataattagatctttttccaagttaattgggttttcccttctcccagt
cataggtggtttttatcatcaagacagactgatattttgtcaggatattt
tcttttacagtgtttgatgtgcataatgccagagttatttttttattatt
cattttctctctttttgttcaatatgagattcaggatcatatttgtttaa
aaggtaacacatagagatgtatgtatatattttgttataagacatacaaa
ataattttaagagggataaaggtgaaaatatcagattctggaaattttaa
gtatctaaactttatacttgtatgatttaccataaacataccaaaacatt
tttctgaaaatttactgtcggtctctgacatgaaaccgtattttgtcagt
agttgaccaagcagttttatgagaactcttctatgcaatgatgca
Rttn rotatin Entrez Gene ID No. 25914

(SEQ ID NO: 60)

cttgctgcctgtctggaaagtgagaatcaaaatgctcagaggattggagc
agctgcccttgggctctgatttacaattatcagaaggcaaaaacagcttt
gaaaagcccatcagtaaaaagaagagtggatgaagcatactccttagcaa
agaaaactttcccaaactcagaagcaaaccctctaaatgcctattatttg
aaatgtcttgaaaacctcgtgcagctccttaattcttcctgagtgccatg
ggatgctacaccttgaagctgacagtcatcaacaggggagctaaagttga
agccagctgtgtgtagcagctgttacctgaagacgtgctacctctctaca
aagtgttgatccccttctttcccatgagagagagaactggtgatactcca
acaccgtccagttgtggcagc
Scoc short coiled-coil protein Entrez Gene ID No.
60592

(SEQ ID NO: 61)

caagagtagatgcagttaaggaagaaaatctgaagctaaaatcagaaaac
caagttcttggacaatatatagaaaatctcatgtcagcttctagtgtttt
tcaaacaactgacacaaaaagcaaaagaaagtaagggattgacacccttc
tgttttatggaattgctgctgatcattttttctttaaaacttggatagat
tccaaaagttacagtacctttgtggcttcattgaatatttatgaagataa
tgtcagatgtagacaaaaataacacaataacaggagacttccataagttt
gtgtattatgttagtctatgaaaacgtgcaaatgtattgtagagacttta
tg
Specc1 spectrin domain with coiled-coils 1 Entrez
Gene ID No. 92521

(SEQ ID NO: 62)

agctgaagactctgaccaagcagatgaaggaggagaccgaggaatggagg
cggttccaggcggatctgcagaccgcagtggtggtggccaatgacatcaa
gtgtgaggcccagcaggagctgcgcaccgtgaagaggaaactgctggagg
aggaggagaagaatgcccggttgcagaaggagctgggggatgtgcagggc
cacggcagggtggtcaccagcagagccgcccctccctccctgggctctgt
cagctagcagagcatttggtggaagaaagacagcccagctcttgccatga
ttgggagccgcagcccatctctagatgaaagggggaatgtgtagaggaga
aattgcctctttataaagagcccagttgtctccttgtgacattctctgtt
ctcagagtcattgccgtcgagtctctgctttttgtccacattttgggatc
agcttactgca
Tpp2 tripeptidyl peptidase II Entrez Gene ID No.
7174

(SEQ ID NO: 63)

gaagagtgcttaaggttgaagtacaatggcacaatctcggctcacctcga
cctctgcttcctgtattcaagtgattcttctgcctcagcctcccgagtag
ctgggattacaggcatgcgccatgacacttggctaattttttngtgtgtt
tttagtagagatggaattttgccatgttggccaggctggtctcaaactcc
tgacctcaagtgatccacccacctcgacctcccaaagtgctggtattata
ggcatgagccaccatgcctggcctcatttatttttaaatagctgcagtaa
tcccggctttagatnaaancacatgaactaataatatcactagtgttca
Vil2 villin 2 (ezrin) Entrez Gene ID No. 7430

(SEQ ID NO: 64)

agagctgagtcatcctagagcaaacctctggagtggagagcgaactactt
cattcccctcccttagcctgggccagagagactccagctctgccttctcc
agccaaaaaatcaaaggcagatgggagaacagccttcagctttggataac
gatgaaatatctggcaccactgatgaatattaaactttctataacc
Znf204 zinc finger protein 204 Entrez Gene ID No.
7754

(SEQ ID NO: 65)

gagcacatatcttacaaaacaccaaaaaattcatagtgaagagaaatcaa
atatacatactgagtgtggggaanccattagacaaaactcttctttttna
caacaataaaancctcacactggagagnttctctgaatgccttaagaatt
tggttaatatggagacccttcccagggaaacagaaggaggatcgtgaaaa
ctgttgactacttagaatgatcacatggtttagtggagagagcatgattc
tgggttttaaaagtcatggatctcaatctcagctcctattactaactaga
tcttttactttggggtaagtcacttcatatctttaggccttaatttcctc
atctgaaaaactggaaggcctgacttgttgagcttta
Znf24 zinc finger protein 24 Entrez Gene ID No.
7572

(SEQ ID NO: 66)

ggcactgtgtaatcattccttgaagtagttggagatggtgctggtatgcc
actgaatgaggtctgagcaggttttcttcacatctgaggggacagtgcca
gccagtcaacttttggggtggggctgaagtctgctgaaaatctgcagttt
tacatgtttcatgggacattcttctgtgcaataaagtttgaga
Amn amnionless Entrez Gene ID No. 81693

(SEQ ID NO: 67)

tactggtgacacttcatggctgcgacccagaatgaacttaatgcacacag
ggacgcagggtgtcactggtcctgggcctttgtccatgactaggtggtca
gcaggacttctgcagctgactgtgcaatggctaaatgaaaagaaggccac
agactaacctccactttcctgtcttcaaaattctagtgacactgggaatg
ctataggacctcctactattctcttaaggtcctaggaaagtttcaggaac
tagggaaaagactgggtactgaggctgtgtccccagatgtctgcttccga
agcagccgcgtcatgacgggtttctgctgaggaagtggtgttggcagggc
cccatatgccctctcgggttgtcaggggtgggagacaggctgtatggggg
tccttcatgtgcagatggaacagcatcgcctcacagctgtgcagacgaac
agatgtggtctactgccacgaacaatgcgg
Ankrd13 bankyrin repeat domain 13B Entrez Gene ID
No. 124930

(SEQ ID NO: 68)

cctcatggtgcctcggagagtggggagcatattgggctgnggtaagcact
agacccaagtagactggacacaaagggctcgcccagggccntggcgccac
ccccaccccttcccaccagctgctgctagcctctgtggttgtacatccca
cttgcccccacacggagactgactctaaaacccttcatccaatggtgnta
acccccggctntcccctgccccacctcacccacccagagaagcacagacc
ccgccaggggcaggggcccaccgcacacccttgtcccgggcctgtctggg
actggccttcccggntcagcnagnnnnnnncagaagggacacaaagaggg
atggaagaaaagaacaaagagaaactgttcctcccacccccttccctgat
gccaggggcaccagactgattctgagg
Ankrd57 ankyrin repeat domain 57 Entrez Gene ID
No. 65124

(SEQ ID NO: 69)

gtcatttaccatggttttcccactgaaggctttaacttttctgataaaat
aatattttaaattttcaaaaacccattcctgaggagaactacttctagca
ttccttttcatgatgtgcttttgtgcagtaagtagcattttcggctactt
aactttacattcctcttatttttcagtttccagtcaagattataaaaagc
aaatgattgatataatttgatattcatagagttgtgcctacctttaatgg
aaaaatacatgtcagatacttagatgtttattgatatgagactatgtggt
taaaaaacccaagtatgtccatgtgtttcttataaggtacacttgaaact
agtgagtgtttgtcacatttcactttcatggtatataaaatgcagtttgc
atatataacttgaatatctggtactagttttttcacgcctgcaatcttgg
agtctaggttgccttgtctct
Apobec4 apolipoprotein B mRNA editing enzyme,
catalytic polypeptide-like 4 (putative) Entrez
Gene ID No. 403314

(SEQ ID NO: 70)

aaatccaagttcctcttactggctttcaaggatcctcctttaacttcctg
ttgccttgtctcatcagcaagatcataagtacctacaggtcaagcactgt
ctctcccttctctttagcttttcccttaggatctagcacattacccagca
aaatgtgagtagcaaggctgaaatgacatctcaataacttcaccaatgat
tgtaactcagcatcccttctccatcccagctgaaagcctgcttcaccatc
ctgcaagagatgttttttctttttgttagcatccattcccctttctaatg
cagctcccaatgcataatgtgtg
Btnl9 butyrophilin-like 9 Entrez Gene ID No.
153579

(SEQ ID NO: 71)

ggtcatcgaatctgcatgcatccctcatacatctggagacttcgtgaagg
ttccagagttactgactgagatttctgagcttttttccccttttctgttt
ggtttcagagtggagagcagcccaaaaatatgcaggtaactgaagccagg
aaactgatttgtgttttggtttggnccggatncttnaaacagaagggagg
tggagagatctgagattagaggacggggctttataggagnccaagtatgg
ggcctgcacacacaagacacacacgcacacttgcaaacacgccacacgac
acatatgcctgcatgtgtatgcacacacatgcacacgtgagctcccaaac
acatcgctccttggggttacactaggtttgtttccatctggcttgaggct
atttgcaggcgagagtgcagagtctgtaatgaacctcccagattctctga
cgaaggggtcccc
C11orf71 chromosome 11 open reading frame 71
Entrez Gene ID No. 54494

(SEQ ID NO: 72)

ttcctgtgtgcttagtcatcgggtccgacacaggctggactgatctgggg
agccgcgaagggcctgccttcacaaagggacgtaacgcaagtactgcggg
cagtgtttgaatatggccctgaacaatgtgtccctgtcctccggtgatca
gaggagcagggtggcctaccgctcttcccatggcgacctcagaccgcggg
cgtcagcgttggcgatggtctccggagacggcttcctcgtttccaggcct
gaagcgattcatctaggacctcggcaggcggtgcgaccaagcgttcgggc
cgagagccgtcgagtggatggtggcggccggagcccaagggaaccagatg
gccggggccggagccgccaagccagattctcaccttacccaatccctgcc
gttgaacccgatctcctaagaagtgtgctgcaacagcgtttgattgcatt
aggaggtgttatcgcagctcgaatttcagtttaaacgaacacctttcctc
tggccctcacttagcttgtgaacag
C14orf145 chromosome 14 open reading frame 145
Entrez Gene ID No. 145508

(SEQ ID NO: 73)

gaaatgcatgggacaggtgactgacatgactgcatcgtggtttagatgta
tagataacacggggaggtgctttacattttaagactttgttcataattct
tttatttatggtttctctgaatcattcttttggaacattctaaaagagcc
agagga
Clorf89 chromosome 1 open reading frame Entrez
Gene ID No. 8979363

(SEQ ID NO: 74)

cgggtgaagagtgtgccggggcggcggctggctgatgggcgcacactgga
cgggcgggctgggctggccgacgttgcccacatactcaatggccttgctg
agcagctgtggcaccaggaccaggtggcggctggcctgcttcccaacccc
ccagagagtgctcctgaatgagtcacgagtggttgcctgtgatcccaccc
ccaaccctcaggtctcgacatagggctggaggctggggcaggaacatgga
tcctatctggaggactggccagcatggcctgatcagggaggatgtggcca
gagaaggcccacccgcgagcagcgctttccttgcagaattcatggcaggg
aggtggggaccaaggccctgagctcgaacatctcccgtggcctttccccc
tttggcagcaccgatggaggatgactgggagagggggtgcctctcaagtt
acttcaatcaagaacctgtattggttgaggtgacaccatctgttgtaaca
g
C20orf7 chromosome 20 open reading frame Entrez
Gene ID No. 779133

(SEQ ID NO: 75)

tgaatgaccttcctagagcacttgagcagattcattatattttaaaacca
gatggagtgtttatcggtgcaatgtttggaggcgacacactctatgaact
tcggtgttccttacagttagcggaaacggaaagggaaggaggattttctc
cacacatttctcctttcactgctgtcaatgacctgggacatctgcttggg
agagctggctttaatactctgactgtggacactgatgaaattcaagttaa
ctatcctggaatgtttgaattgatggaagatttacaaggtatgggtgaga
gtaactgtgcttggaatagaaaagccctgctgcatcgagacacaatgctg
gcagctgcggcagtgtacagagaaatgtacagaaatgaagatggttcagt
acctgctacataccagatctattacatgataggatggaaatatcatgagt
cacaggcaagaccagctgaaagaggttccgcaactgtgtcattt
Ccdc88a coiled-coil domain containing 88A Entrez
Gene ID No. 55704

(SEQ ID NO: 76)

acatatgtacagtatcagtagggaaaatgtaaaaagatgttgttttcttt
tgtcatttaattaggccatctgtcctgttttaaagaaatagttaataatt
caacactttatataacaaatattaactaatacccatatttataaaacatt
tttcagatttaaaagattgttaatacttataaacttagtgttattcttag
aaaaccccatcaaatttaaatgtgatttacacagtgactaggaacatttg
tatttattgtttcttctctgcacttttcatcatctgataaatacaagagc
tcaagtaactgtcttttcttcaagatggcttctatacttgaaatcagtta
atacaatagtttttccagt
Ccne2 cyclin E2 Entrez Gene ID No. 9134

(SEQ ID NO: 77)

gaggaagtcactttactactctaagatatccctaaggaattttttttttt
aatttagtgtgactaaggctttatttatgtttgtgaaactgttaaggtcc
tttctaaattcctccattgtgagataaggacagtgtcaaagtgataaagc
ttaacacttgacctaaacttctattttcttaaggaagaagagtattaaat
atatactgactcctagaaatctatttattaaaaaaagacatgaaaacttg
ctgtacataggctagctatttctaaatattttaaattagcttttctaaaa
aaaaaatccagcctcataaagtagattagaaaactagattgctagtttat
tttgttatcagatatgtgaatctcttctccctttgaagaaactatacatt
tattgttacggtatgaagtcttctgtatagtttgtttttaaactaatatt
tgtttcagtattttgtctgaaaagaaaacaccactaattgtgtacatatg
tattatataaacttaaccttttaatactgtttatttttagcccattgtt
Ceacam6 carcinoembryonic antigen-related cell
adhesion molecule 6 (non-specific cross reacting
antigen) Entrez Gene ID No. 4680

(SEQ ID NO: 78)

gttttaattcaacccagccatgcaatgccaaataatagaattgctcccta
ccagctgaacagggaggagtctgtgcagtttctgacacttgttgttgaac
atggctaaatacaatgggtatcgctgagactaagttgtagaaattaacaa
atgtgctgcttggttaaaatggctacactcatctgactcattctttattc
tattttagttggtttgtatcttgcctaaggtgcgtagtccaactcttggt
attaccctcctaatagtcatactagtagtcatactccctggtgtagtgta
ttctctaaaagctttaaatgtctgcatgcagccagccatcaaatagtgaa
tggtctctctttggctggaattacaaaactcagagaaatgtgtcatcagg
agaacatcataacccatgaaggataaaagccccaaatggtggtaactgat
aatagcactaatgctttaagatttggtcacactctcacctaggtgagcgc
attgagccagtggtg
Chrnb3 cholinergic receptor, nicotinic, beta 3
Entrez Gene ID No. 1142

(SEQ ID NO: 79)

tgatttttgtgaccctgtccatcattgttaccgtgtttgtcattaacgtt
caccacagatcttcttccacgtaccaccccatggccccctgggttaagag
gctctttctgcagaaacttccaaaattactttgcatgaaagatcatgtgg
atcgctactcatccccagagaaagaggagagtcaaccagtagtgaaaggc
aaagtcctcgaaaaaaagaaacagaaacagcttagtgatggagaaaaagt
tctagttgcttttttggaaaaagctgctgattccattagatacatttcga
gacatgtgaagaaagaacattttatcagccaggtagtacaagactggaaa
tttgtagctcaagttcttgaccgaatcttcctgtggctctttctgatagt
gtcagtaacaggctcggttctgatttttacccctgctttgaagatgtggc
tacatagttaccattaggaatttaaaagacataagactaaattacacctt
agacctgacatctggcta
Cllu1 chronic lymphocytic leukemia up-regulated 1
Entrez Gene ID No. 574028

(SEQ ID NO: 80)

cagacacatatacaggaaagaccatgcgaagaagacacagagaggaaacg
gccatctacaatccaaggagagaggcctcagaacaagccaaccctgcaga
taccttgatctaggcatccagaattgtatgaaaatac
Cnnm1 cyclin M1 Entrez Gene ID No. 26507

(SEQ ID NO: 81)

agacaggagggccacagcgtgtggaagtaaagactttggagctagagatg
ccttttccagcaatgattattgacttcaccacaccccttgcctggcctgg
cctgaggctcagcagtgcatgacttctcgtagataacttcacagtcatcc
agtcccaacacctgctcttgcctggtaggaacaggcgaagtgtcagccct
caatgttgggtacttagacccaaaccaataaatggtgagttttgaacaag
aactaccatcatgcaggcttcttgcccagctgaccactggccccggggtg
cctgcctggctggtcttcatcacctgaggccaccaggctcaagccactgc
tgttgcattacacccatccctttgcaaaatccctatggagcctgtcacca
ctcccctccctatatacccccaccccacaaagattttcttcag
Cnot2 CCR4-NOT transcription complex, subunit 2
Entrez Gene ID No. 4848

(SEQ ID NO: 82)

gtgacgttggtaggaaagcatttctttacatggaggtttattttgtggga
cattacctcctctggatgttacttcctcagtttacaagtagtgtaaatnc
tcattgattctnttatgaattgtaanggatttnctcttagcttttgagaa
tttagaatctganatttgaataaaaagtaaatatattcagtataattntn
caaaatgctctagtttcaagatanttaaaanattatgtggaatttacata
attaattcnaaatatagtttgtatttagttccnttatcaaataatgcaaa
tagttggnagatacctcaatttcttttgagtgttaagnaagnagncaagn
aaaggnagtgaagtttttgcaacacattgtgtctttatttggtctgccta
tgtttttatcacattgcttataaaacttttaaaatccttgtttgtataaa
aagtttctttagttaaataaaagtgtgtgtattaattagtgtgccttctg
gacaaattaagaaatattttttctatatttcaatgcggttgtatt
Dhfr dihydrofolate reductase Entrez Gene ID No.
1719

(SEQ ID NO: 83)

tcagatatttccagagaatgaccacaacctcttcagtagaaggtaaacag
aatctggtgattatgggtaagaagacctggttctccattcctgagaagaa
tcgacctttaaagggtagaattaatttagttctcagcagagaactcaagg
aacctccacaaggagctcattttctttccagaagtctagatgatgcctta
aaacttactgaacaaccagaattagcaaataaagtagacatggtctggat
agttggtggcagttctgtttataaggaagccatgaatcacccaggccatc
ttaaactatttgtgacaaggatcatgcaagactttgaaagtgacacgttt
tttccagaaattgatttggagaaatataaacttctgccagaatacccagg
tgttctctctgatgtccaggagga
Eif4a2 eukaryotic translation initiation factor
4A, isoform 2 Entrez Gene ID No. 1974

(SEQ ID NO: 84)

ataccacttagtatagttcgctactattttgtggcctacatgacaggtgt
caagntttttttgaatcaatttttaaaacatgccattgtgtttcaggctc
gcgggattgatgtgcaacaagtgtctttggttataaattatnatcnnnct
ancagtcgttgaaaactatanttcacaggtgnagaagccagcatcttggc
tgtattgaaaaaacttcatacgtttttctactgtgatttgtatgaaaggt
aacatcaaatcaaggaatagattcagtaaagtcagtagtgttcagtaaga
tgatgtaattaaatttgtactagggaaggttgatgagaacaaagtgggaa
aacttgtaaacattgcccagattgtggacatagggttnttttccacnaat
tgttggtcttaccttatgcttgagcttttagtgatgttcttgtgtccatg
tgtttttcttggtgattttttnctatangttgggattttcnttggtgtcg
nctggtagnnnnnnnantgaaccctggtttagttatagtggctttatccc
taaata
Fa2h fatty acid 2-hydroxylase Entrez Gene ID No.
79152

(SEQ ID NO: 85)

ctactcgggcgctcccagaaggagccacctctcagtgcctcacctccccc
tgcctcccagcctccgcagatgaggttcctgccccttcctcctcgtaacc
aaaaccctcactgctcccaggacggtcttatttataaaccagatacatgt
tcttagtctggtcccagaccaaggagctggtcagacggccctttctaatc
ctacatgttgagcttatgtaaaaaatgttgtttcctcctgtttttggttc
ctttcttacccacaaaccattactacttgaaacttaaaaaactcgccaag
tgtaaaggctaaagagaagcagtttgacggaccttgtgatttgtactgtt
tgctgcggagctattta
Fbxo15 F-box protein 15 Entrez Gene ID No. 201456

(SEQ ID NO: 86)

gagaacacctacctcttattggaaaagttggcctctcgtggaaaactgat
atttttgatggctgtataaagagttgttccatgatggacgtaactctttt
ggatgaacatgggaaacccttttggtgtttcagttccccggtgtgcctga
gatcgcctgccacaccctctgacagctctagcttcttgggacagacatac
aacgtggactacgttgatgcggaaggaagagtgcacgtggagctggtgtg
gatcagagagaccgaagaataccttattgtcaacctggtcctttatctta
gtatcgcaaaaatcaaccattggtttgggactgaatattagcagtaggtg
gcaaattattgttgttatttagttgtttatttttgactggctttgttctt
g
Gins4 GINS complex subunit 4 (Sld5 homolog) Entrez
Gene ID No. 84296

(SEQ ID NO: 87)

gccaggctttgtggtatgtacctttagtcccagctactctggaggctgag
gcaggaggatcacttaagccttggaggtcaagaatgcagtgagccattat
catgccactgtgtgaccagaaaccagatgtagccatttcaagcataaaac
atgatatttttgttttccttggactgaaacatagtctgggtcctcaacgt
tgccggtgatgatggttgaacatcatgttttttataaaccttaatttctc
atttaataggaagaaaatctcaggagagccaaaagggaggacctgaaggt
cagcatccaccaaatggagatggagaggatccgctacgtcctcagcagct
acttgcggtgtcgcctcatgaaggtttgacgtggagatacctcaaagtct
ccgacct
Grhl1 grainyhead-like 1 (Drosophila) Entrez Gene
ID No. 29841

(SEQ ID NO: 88)

aaaacactactgcaatcacgtctttngttatgctagtatcagtcagatgc
acttagagtgaagaaacactgtaattacagcacacagattgcaagtattg
cgtaccaagtgatacaactcgaaatgcagttctcatcttcctgttttgag
aaatgattattttatcacgcatcagagccttcgtgctttgattatcttgt
atgttaacaattctagaaaacattcatgaattcacnaaaatangttacta
tggcaggggaacattttgtacacatttaagtatataaaaatactaaaata
tgtaattttataacaaagtcacgggtatctttaggttcagggaactagac
taggtcattcgtgtaaatggactggtagttacagtcttaggttaaagtat
tctaatgaagtatgggaactaaattgctggttttctaag
Gtpbp8 GTP-binding protein 8 (putative) Entrez
Gene ID No. 29083

(SEQ ID NO: 89)

ttctcttgccctttacaaattagttttctcacttaaagctatttttgttt
tttgctttttcactagtgaaaatgttacttcccccatgannnnanntnnn
ntntnntanatctacctgtgaattttgctatatttctttgttggtttggc
tttacaaatatgtagctgtcttctcacatgttacctgctgtaaaaccatt
catttgaatcttaatagctttcacgtttacagtaacaaatgaatttccga
gaaatcaagtaagttgcccaagatccaacatatataataaacatcagaac
tagaacttgaattctgttctttcggttgtttccaacatggactaacacat
tttatcaagaatgttttcaatattcaaataaggactcgaaaaaataggct
tacatagtaacttttatccatcaacttacctatcgatgct
Heatr6 HEAT repeat containing Entrez Gene ID No.
663897

(SEQ ID NO: 90)

agggagatgacactggagcaccccacagcccacaggaaagagaccagatg
gtcagaatggcccttaaacacatgggcagcatccaggcaccaactggaga
cacagccagaagggccatcatgggctttttagaagagatcctggccgttt
gttttgactcatctggatcacaaggggcactcccagggttaacaaatcag
tgaagatcccaccatactttctagatgtcgaaggcggcagtaggaagacc
tgagcttgagcataagatctgtgggatttcatcttaggggcagaaacaat
ccgttcactatttatttagaatgacttagcagccatttaaattttcacag
agggctcaaccacctttggagtgactccatagcactggccatggtcaggg
ttgttggaacatctgacctgtgcatccaggagccgaggagtcaggttgta
atacaggccaagcagacgggctttgagggcattta
Herpud2 HERPUD family member 2 Entrez Gene ID No.
64224

(SEQ ID NO: 91)

aaactaaacatcatatgttctcatatgtccctaagctatgaggatgcaaa
ggcataagaatgatacaatggactttggggactttcagggaaagggtgag
aagggcgtaagggataaaagactacaaattgggttcagtatatactgctc
gggtgatgggtgcnccaaaatcttaaaaatcgccaaagaacttatgtaac
taaataccncctgttccccaaaaaactatggaaattaaaaattaaaaaat
aagtataatttctgctttagcgatattaactattcagtacncaataagtg
agtttagcaattcagtgatt
Ica1 intestinal cell kinase Entrez Gene ID No.
3382

(SEQ ID NO: 92)

catactgcatgctcaggacccatagatgaactattagacatgaaatctga
ggaaggtgcttgcctgggaccagtggcagggaccccggaacctgaaggtg
ctgacaaagatgacctgctgctgttgagtgagatcttcaatgcttcctcc
ttggaagagggcgagttcagcaaagagtgggccgctgtgtttggagacgg
ccaagtgaaggagccagtgcccactatggccctgggagagccagacccca
aggcccagacaggctcaggtttccttccttcgcagcttttagaccaaaat
atgaaagacttacaggcctcgctacaagaacctgctaaggctgcctcaga
cctgactgcctggttcagcctcttcgctgacctcgacccactctcaaatc
ctgatgctgttgggaaaaccgataaagaacacgaattgctcaatgcatga
atctgtacccttcggagggcactcacat
Igl immunoglobulin lambda chain, variable 1 Entrez
Gene ID No. 3535

(SEQ ID NO: 93)

tctggatccaaagacgcttcggccaatgcagggattttactcatctctgg
cctccagtctgaggatgaggctgactattactgtatgatttggcgcggca
ccgctgtggtatttggcggagggac
Il15 interleukin 17 receptor A Entrez Gene ID No.
3600

(SEQ ID NO: 94)

gaagatcttattcaatctatgcatattgatgctactttatatacggaaag
tgatgttcaccccagttgcaaagtaacagcaatgaagtgctttctcttgg
agttacaagttatttcacttgagtccggagatgcaagtattcatgataca
gtagaaaatctgatcatcctagcaaacaacagtttgtcttctaatgggaa
tgtaacagaatctggatgcaaagaatgtgaggaactggaggaaaaaaata
ttaaagaatttttgcagagttttgtacata
Il17rc interleukin 17 receptor C Entrez Gene ID
No. 84818

(SEQ ID NO: 95)

gctgcccgcagaagcgcacatgtgcnnnnnggctncnggtngggacccct
nncanncantgnnccnnncgctttcctgggagaangtcactgtggacnag
gttctcgagttnccattgntgaaaggncncccnnnnnnnnntntnnnnca
ggtgaannnnnnnnnnnnnnngcagctgcaggagtgcttgtgggctgact
ccctggggcctctcaaagacgatgtgctactgttggagacacgaggcccc
caggacaacagatccctctgtgccttggaacccagtggctgtacttcact
acccagcaaagcctccacgagggcagctcgccttggagagtacttactac
aagacctgcagtcaggccagtgtctgcagctatgggacgatgacttggga
gcgctatgggcctgccccatggacaaatacatccacaagcgctgggccct
cgtgtggctggcctgcctactctttnnngctgcgctttccctcatcctcc
ttctcaaaaaggatcacgcgaaagggtggctgaggctcttgaaacaggac
Insc insulin induced gene 1 Entrez Gene ID No.
387755

(SEQ ID NO: 96)

tccttcttactctgcagcaacatggaggagagttttgtgtagtgagtgtg
ggngaagaaatacatttggctgttctcacaccccctctgactatgcacca
gtgaacacatctgagtacataccagctctcctcatcttcttatttatact
taacttatttttgtgtgaaataaatggaggacgaaatcttagagcaacat
catcaaacagtctttggtccttgagaatcttctttgtgttttattttttg
atttctgtagcttttcagttgcagatgttgaaattcgtaatgacaaatat
gacaaattgtcatgggtgattccacttcatcttattttttctactctcac
tatacaatcttgcctcattttttaaaactttggaaccagaggatttcaac
tgcctagca
Ipo11 Intracisternal A particle-promoted polypep-
tide Entrez Gene ID No. 51194

(SEQ ID NO: 97)

gagactacagcagtgttacctgtgcaaatacaacttactacttctgttac
cttgaacttggaaaaaaacagtgctctaccgaatgatgctgcttcaatgt
cagggaaaacatctctaatttgtacacaagaagttgagaagttgaatgag
gcntttgacattttgctagcttttttcatcttagcttgtgttttaatcan
ttttttgatctacaaagttgttcagtttaaacaaaaactaaaggcatcag
aaaactcaagggaaaatagacttgaatactacagcttttatcagtcagca
aggtataatgtaactgcctcaatttgtaacacttccccaaattctctaga
aagtcctggcttggagcagattcgacttcataaacaaantgttcctgaaa
atgaggcacaggtcattctttttgaacattctgctttataactcaactaa
atattgtctataagaaacttcagtgccatggacatgatt
Itch Integrin alpha FG-GAP repeat containing 1
Entrez Gene ID No. 83737

(SEQ ID NO: 98)

gatcattggtatgtcaatctcttgatgaaaaatcagtacctgaatatgtc
tttttgtttttttaagagacagggtcttgctatgttgcccaggcaggatt
tgaactcctgggatcctcccacctcagcctcccgagtacaatacctgaat
tttaaatagagttattgtaagtcttatgaaatgagattttgctgcactct
gacataagataataaaagacagagcaggaattcattattatgagctgctt
gatcagttttaaaccactccatttgatgaaacaagtgaggtccttccctc
ctgaccaggctgtggaatgctgtcttccccaacccccaccccctgcaaaa
gagcagaacaataaggcaattgctcatttt
Itfg1 integrin alpha 2b Entrez Gene ID No. 81533

(SEQ ID NO: 99)

gcagggaaaacacagtccactccccaccaccactccgctccttccacagc
aaatggggactgctcagaaaaccctgtcctttcttttcctctcttcaaag
gcaggcatgtgattgaggatgtgatggtgacttctggcctgttttatttt
gtggtaacttcactttagtcagggaaataattggaatatctttaatctga
tgtagtttgacctttagaaattgaaagtgaaacagctattgttgataatt
cacaaagtattaataaaacttctattacctgtaaaaannnnnnacttaat
ctgcgagaaaactatttagaatattatggaattgtgccatagcttcttta
tttgttcttaattctatattagatttttttttctctgcttcatgaacaag
ttcagattttaaaacattatgcctgaagacatgtccaactttatttttta
tatgttatattctggtccaatatactagagtaataatactctggtattta
ggatatctgtcattgaacctcagttacaaattaaacaatagtagctacca
tatctaagtgat
Kcnmb4 potassium large conductance calcium-acti-
vated channel, subfamily M, beta member 4 Entrez
Gene ID No. 27345

(SEQ ID NO: 100)

agatgagattggttcccagccatttacttgctattttaatcaacatcaaa
gaccagatgatgtgcttctgcatcgcactcatgatgagattgtcctcctg
cattgcttcctctggcccctggtgacatttgtggtgggcgttctcattgt
ggtcctgaccatctgtgccaagagcttggcgatcaaggcggaagccatga
agaagcgcaagttctcttaaaggggaaggaggcttgtagaaagcaaagta
cagaagctgtactcatcggcacgcgtccacctgcggaacctgtgtttcct
ggcgcaggagatggacagggccacgacagggctctgagaggctcatccct
cagtggcaacagaaacaggcacaactggaagacttggaacctcaaagctt
gtattccatctgctgtagcaatggc
Kif14 kinesin family member 14 Entrez Gene ID No.
9928

(SEQ ID NO: 101)

ggatgtttatggtgaaatggcctgtacaagtttaactaagacaacttaac
ttgcattgttaatcaaaaattcttttctcaaagggttaactggttgccat
tttgaatagtatgttcaagggtgtagcttcctgtttctttccaaattata
agtagctacctaaatatagtataattatatattaataatatggcttgctg
gcacagtagtttaccctgttatctgtgtttcataatgggggctgtatgaa
tattatttaaaactaataaaatgttgccagaattatactaaactgttgga
tgagattaggagatcagaggctggaccttctcttgataatgcttgttttg
ttaaaggtataatgaaataatttgtatatgatttgatgaagattaaagac
ccttattttccacagctttaaaaaaaaacctttatttatgatcaagtaat
aaagataatattctacttgtgggatcttacattatggaaatagtttgacg
tttttgacctcaagagtatgtataatttgaagagatactttgtaactatg
cttgggtg
Loc388692 hypothetical gene supported by AK Entrez
Gene ID No. 123662 388692

(SEQ ID NO: 102)

ctcaacatcataaggaatcagacggatgcggaaaccnagncgggntggat
agnaaantctttccaggaaggctccggggcactcaactggtctccaaccn
tcccntgcaacntgtgacgcctgccatnttncccatntttaggcgantgg
caacgcaanccctccgtttgctctgggcaaaacttcgagagttccctctg
aagctggagctttttcctcagatccaagatccaattggtcaccaattcgt
gatttc
Loc401913 hypothetical LOC401913 Entrez Gene ID
No. 401913

(SEQ ID NO: 103)

caagcccattttcctgagggtgaaggggacttttattatataggggcctt
atttggtgggtcagtgctggaggtttacaggctgatcatggcctgtcacc
aggtgatgatgattgaccaagccaaccacatcgaggccctgtggcatgac
gaaagcctcttaaacaagtacctgcttaaccacaaacccactctcccttg
agtacatgtgggattaaaaagtcgatggagtatacgttggatgaatacct
ggtgggtttgtgcaccatgataaactgcaagagatctgtggtcttggtaa
ataacaatgnggaaatgtgaactgatgagggaagcttccagaaagagacc
agagagggggtgattgccagtcagcccgnatcttcctcctgaaatgctac
cctgattta
Loc619208 Entrez Gene ID No. 619208

(SEQ ID NO: 104)

ccttgtcaacatcttcgagcatcggcagctccggangccggggtaactgg
cagcaggtaggaaactatgtgaaagaatctcctgatgtcataatttccgg
gtgtcaccggaacatttgatcatcattcctttggcaattccagccttctg
tggaaaggccagtagaaagcattgatttattcacctctacaggaatcaga
ctcagcctcttttggttttcagtgaagtatgccttttcaatttggaaccc
agccaaggaggtttccagtggaaggaggagattcttcaattgagctggaa
cctgggctgagctccagtgctgcctgtaatgggaaggagatgtcaccaac
caggcaactccggaggtgccctggaagtcattgcctgacaataactgatg
ttcccgtcactgtttatgcaacaacgagaaagccacctgcacaa
Loc645513 Similar to septin 7 Entrez Gene ID No.
645513

(SEQ ID NO: 105)

tgaacagagtaacaggactatatttctgaaaaaggatgaacttactggaa
acaggattgcgtgcctgaaatatacccaaatggatataaatgtcaactca
gttctgggctggagatatagatttggaatgcaccaacaatgcagatggta
atcctggcatgcgagtag
Loc654342 Similar to lymphocyte-specific protein 1
Entrez Gene ID No. 654342

(SEQ ID NO: 106)

ttctagaatctcagcttcttaaatcaagaaattgtgtcttgttcatgtct
gaattccccaagtgaacacagtgagtgggtgcttgacaaatctttgntgg
tnangngcnaaaaaaggggattctgtgcccaataccatgaaatcaatgca
cagaagattcantcaatcaagaaaggtgcacagacactggccacacacac
tgacatttgtttgcagatgttccagtccccctgacttccaccaatccatt
cattcattccacaagcatttttgctggggtggaagcagggccatacaggg
tgtgtacatcacagatagggtgggtttgtataatgagtataaaaaacttc
tagcagaagatgacaaagtatatcaagaaagggctctcttcgaaatcaca
Lrrc37a leucine rich repeat containing 37A Entrez
Gene ID No. 9884

(SEQ ID NO: 107)

ggctttgggagtgagcagctagacaccaatgacgagagtgatgttatcag
tgcactaagttacatttgccatatttctcagcagtaaacctagatgtgga
atcaatgttactaccgttcattaaactgccaaccacaggaaacagcctgg
caaagattcaaactgtaggccaaaaccggcaaaaagtgaatagagtcctc
atgggcccaatgagcatccagaaaaggcacttcaaagaggtgggaaggca
gagcatcaggagggaacagggtgcccaggcatctgtggagaacgctgccg
aagaaaaaaggctcgggagtccagccccaagggagctggaacagcctcac
acacagcaggggcctgagaagttagcgggaaacgccatctacaccaagcc
ttcgttcagccaagagcataaggcagcagtctctgtgctgacacccttct
ccaagggcgcgccttctacctccagccctgcaaaagccctaccacaggtg
agagacag
Mrp130 mitochondrial ribosomal protein L30 Entrez
Gene ID No. 51263

(SEQ ID NO: 108)

ttatggctgggattttgcgcttagtagttcaatggcccccaggcagacta
cagaccgtgacaaaaggtgtggagtctcttatttgtacagattggattcg
tcacaattcaccagatcaagaattccagaaaaagtgtttcaggcctcacc
tgaagatcatgaaaaatacggtggggatccacagaaccctcataaactgc
atattgttaccagaataaaaagtacaagaagacgtccatattgggaaaaa
gatataataaagatgcttggattagaaaaagcacatacccctcaagttca
caagaatatcccttcagtg
Nipsnap3 bnipsnap homolog 3B (C. elegans) Entrez
Gene ID No. 55335

(SEQ ID NO: 109)

gttaatttgctgtgcttcttgcatttttgaaagttacatattctccactg
ctttaagaaataattcagttcactttcaccttggcatttcagtatctgtt
acacattagaagtagttgtcactatttcatc
Parp2 poly (ADP-ribose) polymerase family, member
2 Entrez Gene ID No. 10038

(SEQ ID NO: 110)

gccatgggcttcgaattgcccaccctgaagctcccatcacaggttacatg
tttgggaaaggaatctactttgctgacatgtcttccaagagtgccaatta
ctgctttgcctctcgcctaaagaatacaggactgctgctcttatcagagg
tagctctaggtcagtgtaatgaactactagaggccaatcctaaggccgaa
ggattgcttcaaggtaaacatagcaccaaggggctgggcaagatggctcc
cagttctgcccacttcgtcaccctgaatgggagtacagtgccattaggac
cagcaagtgacacaggaattctgaatccagatggttataccctcaactac
aatgaatatattgtatataaccccaaccaggtccgtatgcggtacctt
Pbrm1 polybromo 1 Entrez Gene ID No. 55193

(SEQ ID NO: 111)

gtcagcagtcagcaaattaacatcatcatactcttccatttttagtttct
gttggattttcatcaagtcaatgggctgagaaaccacttcataatagtct
ggttgatttcttcgctttggtgccctaatgaagagctcacagagaagtct
gccctgttcatccttatagtctcggatggtattatagagttcatggcaca
cggcaataggatctacagttggaagattggaaagtctcctccttttcctg
cttgggcctggtgttgacacagaatggtgcccatcatcaaagtccccgct
gacactgctggaaggggaggtagctcttcttctct
Pex13 peroxisome biogenesis factor 13 Entrez Gene
ID No. 5194

(SEQ ID NO: 112)

ggatgaccatgtagttgccagagcagaatatgattttgctgccgtatctg
aagaagaaatttctttccgggctggtgatatgctgaacttagctctcaaa
gaacaacaacccaaagtgcgtggttggcttctggctagccttgatggcca
aacaacaggacttatacctgcgaattatgtcaaaattcttggcaaaagaa
aaggtaggaaaacggtggaatcaagtaaagtttccaagcagcaacaatct
tttaccaacccaacactaactaaaggagccacggttgctgattctttgga
tgaacaggaagctgcctttgaatctgtttttgttgaaactaataaggttc
cagttgcacctgattccattgggaaagatggagaaaagcaagatctttga
tatctttcatgtttgcctgc
Phlda1 pleckstrin homology-like domain, family A,
member Entrez Gene ID No. 122822

(SEQ ID NO: 113)

gaagtgggacgagcacatttctattgtcttcacttggatcaaaagcaaaa
cagtctctccgccccgcaccagatcaagtagtttggacatcaccctactg
aaaacttgcgattcttcttagttttctgcatacttttcatcacgatgcag
gaaacgatttcgagtcaagaagacttttatttatgaacctttgaaaggat
cgtcttgtatggtgaattttctaggagcgatgatgtactgtaattttatt
ttaatgtattttgatttatgattatttattagttttttttaaatgcttgt
tctaagacatttctgaatgtagaccattttccaaaaaggaaactttattt
tcaaaaacctaatccgtagtaattcctaatcttggagaataaaaaagggc
ggtggaggggaaaacattaagaatttattcattatttctcgagtactttc
agaaagtctgacactttcattgttgtgccagctggtt
Pol3s polyserase 3 Entrez Gene ID No. 339105

(SEQ ID NO: 114)

cagaccctgttccttcgaggaatggggagggagggagggaccaaagccgt
gaggatgaggacaactccaccctccttccttccccacaggccaaccaacc
agctgctgacaggggacctggccattctcaggacaagagaatgcaggcag
gcaaanngcattactgcccctgtcctnccccaccctgtcatgtgtgattc
caggcaccagggcaggcccagaagcccagcagctgtgggaaggaacctgc
ctggggccacaggtgcccactccccaccctgcaggacaggggtgtctgtg
gacactcccacacccaactctgctaccaagcaggcgtctcagctttcctc
ctcctttaccctttcagatacaatcacgccagccacgttgttttgaaa
Pparbp PPAR binding protein Entrez Gene ID No.
5469

(SEQ ID NO: 115)

ggcttaggcctcaaatggcttcttctaaaaactatggctctccactcatc
agtggttccactccaaagcatgagcgtggctctcccagccatagtaagtc
accagcatataccccccagaatctggacagtgaaagtgagtcaggctcct
ccatagcagagaaatcttatcagaatagtcccagctcagacgatggtatc
cgaccacttccagaatacagcacagagaaacataagaagcacaaaaagga
aaagaagaaagtaaaagacaaagatagggaccgagaccgggacaaagacc
gagacaagaaaaaatctcatagcatcaagccagagagttggtccaaatca
cccatctcttcagaccagtccttgtctatgacaagtaacacaatcttatc
tgcagacagaccctcaaggctcagcccagactttatgatt
Prkd2 protein kinase D2 Entrez Gene ID No. 25865

(SEQ ID NO: 116)

gggagagggaggagtaatggaggaggagttggaaactggggagagatgga
aggaatgtgactggagggtagagaacttggagaa
Prr7 proline rich 7 (synaptic) Entrez Gene ID No.
80758

(SEQ ID NO: 117)

gaatcggacatgtccaaaccaccgtgttacgaagaggcggtgctgatggc
agagccgccgccgccctatagcgaggtgctcacggacacgcgcggcctct
accgcaagatcgtcacgcccttcctgagtcgccgcgacagcgcggagaag
caggagcagccgcctcccagctacaagccgctcttcctggaccggggcta
cacctcggcgctgcacctgcccagcgcccctcggcccgcgccgccctgcc
cagccctctgcctgcaggccgaccgtggccgccgggtcttccccagctgg
accgactcagagctcagcagccgcgagcccctggagcacggagcttggcg
tctgccggtctccatccccttgttcgggaggactacagccgtatagaggg
gcgcccggcgccccgggccccaccggcggactcctggcctgactgcgggg
ctttttaaatgcttccctggactgcggggaggggcggggggagggaggga
tttcttatcccgtttgttacatt
Psph phosphoserine phosphatase Entrez Gene ID No.
5723

(SEQ ID NO: 118)

ttttctactcagcagatgctgtgtgttttgatgttgacagcacggtcatc
agtgaagaaggaatcggatgctttcattggatttggaggaaatgtgatca
ggcaacaagtcaaggataacgccaaatggtatatcactgattttgtagag
ctgctgggagaaccggaagaataacatccattgtcatacagctccaaaca
acttcagatgaatttttacaagttacacagattgatactgtttgcttaca
attgcctattacaacttgctataaaaagttggtacagatgatctgcactg
tcaagtaaactacagttaggaatcctcaaagattggtttgtttgttttta
actgtagttccagtattatatgatcactatcgatttcctggagagttttg
taatctgaattctttatgtatattcctagctatatttcatacaaagtgtt
ttaagagtggagagtcaattaaacacctttactcttaggaatatagattc
ggcagccttcagtgaatatt
Rfx3 regulatory factor X, 3 (influences HLA class
II expression) Entrez Gene ID No. 5991

(SEQ ID NO: 119)

tagagctgaatattacttgattacaaatcagattgcttaagggtgtggaa
tagcaggctagttttaataccaacttgttaacataaaatcatatatgttt
taganccattcttatttagttacaattttagaaagttaacaaagtaagca
ggtacttatcgaagtgcatcttttcagtctaaatgtttgtctgtgtgtct
aggtgctggtgagtccacatggacacatgnagnnnccatggggcaggagt
ctgctataaagtcagaaggtgagatcctagagagttacacccagccccat
tttaatttgcatgaaaagccaaggttcttttaagcactcaaattatttaa
tgnttaaaacacaagaaaggcacatctgttcatttaaat
Rps16 ribosomal protein S16 Entrez Gene ID No.
6217

(SEQ ID NO: 120)

agagggccttggagtgacaccctgacccccatccactagtacttganggc
cagtggtggcagaagccacagaaacaagaagcccagtgagatggctaagc
tgcccagcatgtaacttaaatccctgttcattccccattcctttagctgc
tggagccagttctgcttctcggcnaggagcgatttgctggtgtagacatc
cgtgtccgtgtaaagggtggtggtcacgtgggcccngatttatnnnagtc
ccanaactgggcgcatggaggaggtggctctgggagggaggccttcacag
cgctcctgtaccctttaattgtgtgtctttctcacagctatcc
Samd4 asterile alpha motif domain containing 4A
Entrez Gene ID No. 23034

(SEQ ID NO: 121)

ggtgaatgtgtattcctctgggaggaataggaagaaaacaggaatgttaa
taatgtcgaacagaaaacttcctcccttattaatatataatcctcatgta
tttatgcctaatgtaagctgacttttaaaaagctttcttttgttgcatgc
cctgtgcaggcatctgtattgtacatgcatgcctttcgtcctgttttcct
gtataaagttagtgaacaaagaaatatttttgcctagttcatgttgccaa
gcaatgcatattttttaaatttgtcatatatggaaagagcatgtttgtta
catgtaaaagctttactgatatacagatatactaatgtttgaagatgctg
ttctttgcaagtgtacagttttcaaatgttgttaccagtgaaacaccctt
gtggtttaaacttgctacaatgtatttattattcatttcctcccatgtaa
ctaagaa
Scamp1 secretory carrier membrane protein 1 Entrez
Gene ID No. 9522

(SEQ ID NO: 122)

tcagttcatatctttctgggcttgacatggctgatggtgtagctgaaacc
ctcctaacactaaaagccatttaatcttttctgtaataggagcagaaaat
agttaatcatccacctagtaatataagattactgtgaatattatcttcta
tacattaaaacagttctagtttgtagaataataccatacaagttttattt
ttaaattctagttattttcagtgcttacttaaatgtaattctagaattcc
tccacaacttttaatattttgtatgccagtgattctcaagataaatcatg
attgtagtagttgttactgttggcagtttgtagtagtattcaggtatttt
ggggatgggggaaaacaccaaaaatcagtgtcttttatctggtgatcact
gtggtatctacagtattctagtctcctgcacaaaaactgaacccactggg
cc
Scn11a sodium channel, voltage-gated, type XI,
alpha Entrez Gene ID No. 11280

(SEQ ID NO: 123)

ccaaccatgatgaaactccgtctctactaaaatacaaaaattagctgggc
atggttgcgtgcgcctgtagtcccagctacttgggaggctgaggcaggag
aatcgcttaaacctgggagacggaggttgcagtgagccaagatcgtgtca
ctgcactccagcctggtgacagagtgagactctgtttcaaaaaagaaaag
aaaagaaacatggttcaaattatatctaaacaaaaaagaataagaaacaa
aaaacacattaaaattttaagttgtattttctatgtttctagatacatca
tttttgtttgatattttcctgatgcaagtatgtggtttatcacatgtagc
tcttttgcatgctaaatgaaaattcaagacttgccaataaatgaatagct
tattgcagacattttttaccaacattaattattttgggtttgtttaaaac
ctagaggcacaatcttgacttgtcaattactaccctttcacaagctacca
tctcagatatatatatatatataaattcaataaagctttctgtttgtgtt
c
Sdccag8 serologically defined colon cancer antigen
8 Entrez Gene ID No. 10806

(SEQ ID NO: 124)

cttcacaatagcaaacgtaaacgatggaattgatggaatcaaccgaaatt
gacggaatcaatctaaatgttcatcactgacagattgtgtaaagaaaatg
tggaacatggacaccatggaatagtatgcagccataaaaagaatgagatc
cgatcttttgcaggaacatgcatggagccggagacagttatccttagcaa
actaacgcaggaagagaaagccaaatactgcatattcttacgtataagtg
ggagctaaatgataagaacttatgagcacaaagtaggaaaccacagacag
tggcatctccttgaggatatagggtgggagcagggagaggagcagaagag
atcactattgggtactgggcttaatacctgggtgataaaataatctgtat
aacaaaaccccgtgacatga
Sephs1 selenophosphate synthetase 1 Entrez Gene ID
No. 22929

(SEQ ID NO: 125)

aaaggtgttctctgtgttatgtaaagtggaggcttccttatattttaacc
tactaagcaatgaggagggattcctgtcattaagcacaagggcgctggat
cctcaagtgcccatcttcgtgagagaaaaagcagcacatcctgcccattt
ctggtgctttctgctcacaggcaccaaagctgcacatgtaaactgacttc
ttgccaaaggaaatgacccctgggaagttcaagctcctggaagaggcttt
aactcggacgcgccctcctccaggaaccagtgggcagggcagccttcatg
catgtgtaactggacctccagccataagcatggtgtgcagtatggaagag
cctgctacggaactgaaagtgattggacattttataggaattgatagaga
tgttggtcctcaaaagctaca
Slc2a13 solute carrier family 2 (facilitated
glucose transporter), member 13 Entrez Gene ID No.
114134

(SEQ ID NO: 126)

aacaacattattccatctcatttaaaggttnaaaaagaagagacaactct
agccnaagtagaaatttatattctacacgtccaaactgtctcctagcagc
ttttggactatatatcacttgatgttaaagtatcttttatttgtaataaa
tattcaaatttctatttagaagctctaatgtatacctagattaaatcaaa
tcacagttttatgcttttaaaatatatgtatttcaaactgtatattttaa
tttctgagtgcatgttatatagtatttaatacttcagatgtcttggcaaa
ttcaatataagtatttattcccacaagcgatatatgggatatctcttaaa
aattatgaatatgtaccatttccttcaaagtcatcctagcctatgctgta
tcaaaagtattgtatattttatggagatttagtgatatacatgtaaatgt
tttttaagttattttattgaagttcaatctttacataaaattaaaatctt
tttttaaaaaaagtgtcagtgccagaactgtaa
Slc30a5 solute carrier family 30 (zinc trans-
porter), member Entrez Gene ID No. 564924

(SEQ ID NO: 127)

tgttcataaacatttgagcaccatgaaatcaaaataccctataactactt
tctatagtcatatctaatttatatttttttcatttccanttgtaactaga
tatgtagtaaagtctgaaaagactttaccatagacaataacatgcagttt
tatcagcaccaaagaatgttgtccaaaagaaactttttaatacctgtctt
tctatttataacatctgaatattttcattcttatattaagaattttgata
agtagattgaatttagtatgagtactattttcttatatataccacaatgg
caaacatgtattataaatcatatttttgtcttaccaattttaatatatga
ggggttttagaaatttgttgtaagttatttttatattccttgtcttttgc
atattttttggccaaaatcttcaatacat
Spa17 sperm autoantigenic protein 17 Entrez Gene
ID No. 53340

(SEQ ID NO: 128)

ttcgaggagcaagaaccacctgagaaaagtgatcctaaacaagaagagtc
tcagatatctgggaaggaggaagagacatcagtcaccatcttagactctt
ctgaggaagataaggaaaaagaagaggttgctgctgtcaaaatccaagct
gccttccggggacacatagccagagaggaggcaaagaaaatgaaaacaaa
tagtcttcaaaatgaggaaaaagaggaaaacaagtgaggacactggtttt
acctccaggaaacatgaaaaataatccaaatccatcaaccttcttattaa
tgtcatttctccttgaggaaggaagatttgatgttgtgaaataacattcg
ttactgttgtgaaaatctgtcatgagcatttgtttaataagcataccatt
gaaacatgccacttgaagatttctctgagatcatgagtttgtttacactt
gtctcaagcctatctatagagacccttggatttagaattatagaactaaa
gtatctgagattacagagatctcagaggttatgtgttctaactattatc
Tcf7l2 transcription factor 7-like 2, T-cell
specific, HMG-box Entrez Gene ID No. 6934

(SEQ ID NO: 129)

gaaatggccactgcttgatgtccaggcagggagcctccagagtagacaag
ccctcaaggatgcccggtccccatcaccggcacacattgtctctaacaaa
gtgccagtggtgcagcaccctcaccatgtccaccccctcacgcctcttat
cacgtacagcaatgaacacttcacgccgggaaacccacctccacacttac
cagccgacgtagaccccaaaacaggaatcccacggcctccgcaccctcca
gatatatccccgtattacccactatcgcctggcaccgtaggacaaatccc
ccatccgctaggatggttagtaccacagcaaggtcaaccagtgtacccaa
tcacgacaggaggattcagacacccctaccccacagctctgaccgtcaat
gcttccatgtccaggttccctccccatatggtcccaccacatcatacgct
acacacgacgggcattccgcatccggccatagtcacaccaacagtcaaac
aggaatcgtcccagagtgatgtcggc
Tmem30b transmembrane protein 30B Entrez Gene ID
No. 161291

(SEQ ID NO: 130)

tatactcactcaaggcagtgcaagatcttgaagtactttttagcagttaa
gtaatattgaattgtattgaatagtttacatagtttattctagtctttga
aaattactgaacatggacaatgtgcatgtcattgacatctgccttagaac
ttctgggacaatcctgattcgagagattctatcccattatttacatatac
caaaaatactttgttaatttaatgtgttggcttcccaactcctgaacacg
acacaattttattattagattttgtatggtgattttaggctatgaaaaca
tgatcattatatgtatatagatacatttttatttgttacaaatgtttgag
cagctcactagcccacccctcctctattttgggtaagagaatttactacc
ttttttaactatgtagttgagagcaacatgtattttgttatttttagaat
ggtcagtatattgctataaaattttaaatgagactatgaaagttaaagta
ttctgattctggttaaattaacgaatatggttccaggccctgt
Trpm7 transient receptor potential cation channel,
subfamily M, member 7 Entrez Gene ID No. 54822

(SEQ ID NO: 131)

gttgcagtgatgacttttgtgaaacaaaaacttatgtatcattttagtga
tactcttaagattatttgttttttggcagtaaatgtgaaaattctttgtt
gttctactttatgaatagaacttaaggaaataactccaaaacaatgtaat
ttgtataagaaggttcataaaaatcctgtaaggtttaaattagtttagaa
gaaaaataatagtttgctgtaactttttctccctaaagaaacaaggtcca
actaatccaatgctgtttcatcttgttcgagacgtcaaacaggtaagaga
ttattttttgcttttga
Wbscr16 Williams-Beuren syndrome chromosome region
16 Entrez Gene ID No. 81554

(SEQ ID NO: 132)

agagctgagtcatcctagagcaaacctctggagtggagagcgaactactt
cattcccctcccttagcctgggccagagagactccagctctgccttctcc
agccaaaaaatcaaaggcagatgggagaacagccttcagctttggataac
gatgaaatatctggcaccactgatgaatattaaactttctataacc
Wdr20 WD repeat domain 20 Entrez Gene ID No. 91833

(SEQ ID NO: 133)

ggagactgtctcactgatgttgatttctttattcatttccgcatctgtta
cacgaacttcgtgtcataaattgctatcctttcatttgaaagtgtaaaaa
atttcctgcatttttatcatttctgtatacttgagtttattagagattgt
tatgttaggcgacactgtataaaattgtatggatattttgagtgaaaatc
aaaagtaaaattcacatgtatttccttttttatattttcatccaatttct
tgacaacttgaataaatttcataaagagccttcctaa
Wdr55 WD repeat domain 55 Entrez Gene ID No. 54853

(SEQ ID NO: 134)

gcaagctctcattggctctgagcgcgaccccgcctcccaggggggtggag
gtatccactgcacgtgcgccgcccgggcttcgctcagaccttcaggtgaa
agctgcaaagtcgcgggtgcgtatgtacgggggctgcctcccgaggagga
gctcccaagccgcagggtggacgctggagacaagaacctcagggtcacaa
gtttactgtttttctcccttttccatccctacattggtctgctggggaag
gcggggctaggcatcactgacacacgcagactccgtggttgaggcatttt
attggacctttggcaattggtggtggggaggcatctgctccaactggtgc
ggggccctgcagatgggaccatctcaggctgggtccttgtagcccaggag
cacagactggactaagcctcctgggccttgtatgaaaaaggtgttgtacc
tggccgtttttgccagt
Znf492 zinc finger protein 492 Entrez Gene ID No.
57615

(SEQ ID NO: 135)

actccgtcctgggtgacaaagtgagactccctctcaaaaantaaatangt
aaataaantaaatggtggtaacnatacnctatttggtaaannnnnncnct
aacatctgtagtactaatcttttttccagtggctttaaactgcaaataag
gaatgttgtttctgtaggtaaaatttttatttattttttcccatttaaat
ttacttttgttagttttttcaggcatataatatttatgttatatatggca
tattctgataagaggcatacaatatgtaataatcacattagggtaaatga
ggtatccatcacctttagaatttattttttgtattatgaacagttcaatt
gtacagttttagtttttttaaaatatacgattgttattgactacagggt
Znf502 zinc finger protein 502 Entrez Gene ID No.
91392

(SEQ ID NO: 136)

tgataagactcagaacaggaagcctgcatgtgactgagcaagtcacctac
ataaccctgcctgtactaaggtgtacacctgtctattgtaagtttgccta
ggctgttggtgtacagagaccagaggagagagacacactaggactaacaa
tgtcctaacaaaatggtacttagtttgttggtctttaggagaaagcatta
gtaatgaaagaagaaagaattttcacttggttggacattggggctgctta
agaaagttgacatttgtcgtggaatgactttggaaagacttctaaaagaa
tctttttcaaatccctgaaaatcaggatagcacattttgctactgactgt
gacagtgttttattcttttgagagaaatgacatagttttccctttatttc
ccaaattcctttcatgttcttaactgctacccagaaattgagcttcagaa
gattgaggatagcctttgattggtattta
Znf557 zinc finger protein 557 Entrez Gene ID No.
79230

(SEQ ID NO: 137)

agtagatcttaccttgctgttcataagagaatccacaatggggagaaacc
ctatgaatgcaatgactgtgggaaaaccttcagcagcagatcttacctta
ctgttcataagagaatccacaatggggagaaaccctacgaatgcagtgac
tgtgggaaaaccttcagcaattcctcatacctcagaccgcacttgagaat
tcacactggagaaaaaccgtacaaatgtaaccagtgttttcgtgagttcc
gcactcagtcaatcttcacaaggcacaagagagttcatacgggggagggt
cattatgtatgtaatcagtgtggaaaggctttcggcacgaggtcatctct
ttcttcgcactatagcattcatacaggggagtacccttacgaatgccacg
attgtgggagaaccttcaggaggaggtcgaatctgacacagcacataaga
actcatact
Znf576 zinc finger protein 576 Entrez Gene ID No.
79177

(SEQ ID NO: 138)

aagctgttgacagggctgcttttctttttggaggctctaggggagcgtct
ttctttgcccttccagcttctagaagctgcccaaattctgtggtttgggg
cctcctttcaaaaccagcaatggccaatcagtcttacatcactcaaacac
ttgagtgttctgtctccctcttccatgtttgaggacccttgtgattacac
tgtgaaaacccagataagccaggataatctccctatcttattatgaggca
agtatgttaagattttattctataatcagagaatcttatgctatgattgt
tatatgtgagcattatagatgctcttgaaatgttaaaatcacatcagcac
tggaaaataactcctaaatgtccaaaaagaacatgagatttatggtgctt
gaaatgttgctaaacgtaaatttgtatctattctgaaattatataaatta
acctacctggccaggca

1. A method of diagnosing a mood disorder, the method comprising:
(a) determining the expression of a plurality of biomarkers for the mood disorder in an isolated sample from the individual, the plurality of markers selected from the group of biomarkers listed in Tables 3 and 7; and
(b) diagnosing the presence or absence of the mood disorder based on the expression of the plurality of biomarkers.

2. The method of claim 1, wherein the plurality of biomarkers comprise a subset of about 10 markers designated as Mbp, Edg2, Fzd3, Atxn1, Ednrb for high mood and Fgfr1, Mag, Pmp22, Ugt8, Erbb3 for low mood.

3. The method of claim 1, wherein the plurality of biomarkers comprise a subset of about 10 markers designated as Edg2, Ednrb, Vil2, Bivm, Camk2d for high mood and Trpc1, Elovl5, Ugt8, Btg1, Nefh for low mood. This panel is derived from the meta-analysis.

4. The method of claim 1, wherein the plurality of markers comprise a subset of about 20 biomarkers designated as Mbp, Edg2, Fgfr1, Fzd3, Mag, Pmp22, Ugt8, Erbb3, Igfbp4, Igfbp6, Pde6d, Ptprm, Nefh, Atp2c1, Atxn1, Btg1, C6orf182, Dicer1, Dnajc6, and Ednrb.

5. The method of claim 1, wherein the plurality of markers comprise a subset of about 10 markers for high mood designated as Mbp, Edg2, Fzd3, Atxn1, Ednrb, Pde9a, Plxnd1, Camk2d, Dio2, Lepr, and a subset of about 10 markers for low mood designated as Fgfr1, Mag, Pmp22, Ugt8, Erbb3, Igfbp4, Igfbp6, Pde6d, Ptprm, and Nefh.

6. The method of claim 1, wherein the mood disorder is bipolar disorder or depression (major depressive disorder).

7. The method of claim 1, wherein the sample is a bodily fluid.

8. The method of claim 1, wherein the sample is blood.

9. The method of claim 1, wherein the level of the marker is determined in a tissue biopsy sample of the individual.

10. The method of claim 1, wherein the level of the marker is determined by analyzing the expression level of RNA transcripts.

11. The method of claim 1, wherein the expression level of the marker is determined by analyzing the level of protein or peptides or fragments thereof.

12. The method of claim 1, wherein the expression level is determined by an analytical technique selected from the group consisting of microarray gene expression analysis, polymerase chain reaction (PCR), real-time PCR, quantitative PCR, immunohistochemistry, enzyme-linked immunosorbent assays (ELISA), and antibody arrays.

13. The method of claim 1, wherein the determination of the level of the plurality of biomarkers is performed by an analysis of the presence or absence of the biomarkers.

14. (canceled)

15. (canceled)

16. A method of predicting the probable course and outcome (prognosis) of a mood disorder, the method comprising:
(b) analyzing the presence or level of a plurality of markers of the mood disorder in a test sample, the markers selected from the group consisting of markers listed in Tables 3 and 7; and
(c) determining the prognosis based on the presence or level of the markers and one or more clinicopathological data to implement a treatment plan.

17. The method of claim 16, wherein the treatment plan is for a high mood disorder if the molecular markers selected from the group consisting of Mbp, Edg2, Fzd3, Atxn1, and Ednrb are present.

18. The method of claim 16, wherein the treatment plan is for a low mood disorder if the molecular markers selected from the group consisting of Fgfr1, Mag, Pmp22, Ugt8, and Erbb3 are present.

19. The method of claim 16, wherein the treatment plan for a high mood disorder comprises administering a pharmaceutical composition selected from the group consisting of divalproex, lithium, lamotrigene, carbamazepine, topiramate.

20. The method of claim 16, wherein the treatment plan for a low mood disorder comprises administering a pharmaceutical composition selected from the group consisting of fluoxetine, sertraline, citalopram, duloxetine, venlafaxine and buproprion.

21. The method of claim 16, wherein the clinicopathological data is selected from the group consisting of patient age, previous personal and/or familial history of the mood disorder, previous personal and/or familial history of response to mood disorder, and any genetic or biochemical predisposition to psychiatric illness.

22. The method of claim 16, wherein the test sample from the subject is of a test sample selected from the group consisting of fresh blood, stored blood, fixed, paraffin-embedded tissue, tissue biopsy, tissue microarray, fine needle aspirates, peritoneal fluid, ductal lavage and pleural fluid or a derivative thereof.

23. (canceled)

24. (canceled)

25. The method of claim 16, wherein the treatment plan is a personalized plan for the patient.

26. (canceled)

27. (canceled)

28. (canceled)

29. (canceled)

30. (canceled)

31. (canceled)

32. (canceled)

33. (canceled)

34. (canceled)

35. A method of diagnosing bipolar mood disorder using blood biomarkers, the method comprising analyzing expression profile of a plurality of biomarkers selected from the group consisting biomarkers listed in Tables 3 and 7 whose expression levels in a blood sample is associated with an increased risk of bipolar disorder.
